(19) United States
(12) Patent Application Publication
(10) Pub. No.: US 2024/0271204 A1
(43) Pub. Date: Aug. 15, 2024

(54) METHOD AND KIT FOR DETECTING EDITING SITES OF BASE EDITOR

(71) Applicant: PEKING UNIVERSITY, Beijing (CN)

(72) Inventors: Chengqi YI, Beijing (CN); Zhixin LEI, Beijing (CN); Haowei MENG, Beijing (CN); Zhicong LYU, Beijing (CN)

(21) Appl. No.: 18/562,762

(22) PCT Filed: May 20, 2022
(86) PCT No.: PCT/CN2022/094072

§ 371 Date: Nov. 20, 2023

(30) Foreign Application Priority Data
May 20, 2021 (CN) 202110551156.9

(51) Int. Cl. C12Q 1/6869; C12Q 1/44; C12Q 1/48

(52) U.S. Cl. CPC C12Q 1/6869; C12Q 1/44; C12Q 1/485

(57) ABSTRACT

Provided are a method for detecting nucleic acid sites edited by a base editor, and a kit for implementing the method. Also provided is a method for detecting the editing efficiency or off-target effects of the base editor editing nucleic acids.

### TECHNICAL FIELD

The present application relates to the technical field of gene editing (especially base editing). Specifically, the present application relates to a method for detecting a site where a base editor (e.g., a single base editor or a dual base editor) edits a nucleic acid, and a kit for implementing the method. The present application also relates to a method for detecting the editing efficiency or off-target effect of a base editor (e.g., a single base editor or a dual base editor) editing a nucleic acid.

### BACKGROUND ART

In 2016, David Liu et al. fused rAPOBEC1 from rats with nCas9 (D10A) based on the CRISPR/Cas9) system, and developed a cytosine base editor (CBE) (Komor, et al. Nature 533, 420-424, doi:10.1038/nature17946 (2016)). The editing principle of the cytosine base editor as designed is as follows: first, nCas9) that has lost part of its nucleic acid cutting activity can still be guided by sgRNA, driving rAPOBEC1 connected to nCas9 to a desired target site; then, sgRNA will form an R-loop structure with the DNA sequence of the desired gene, so that the non-sgRNA complementary DNA (non-target strand) in the single-stranded state in the R-loop can be bound by APOBEC1, and cytosine (C) in a certain range of the chain can be deaminated into uracil (U); finally, these uracils can be completely converted to thymine through the subsequent DNA replication process, thereby finally realizing the base conversion from C to T. Since then, a variety of new CBE editing systems have been developed in succession with different degrees of optimization in terms of editing efficiency, active editing window width, and editable sequence range, for example, YE1-BE, BE4max, etc. (Kim, Y. B. et al. Nature biotechnology 35, 371-376, doi:10.1038/nbt.3803 (2017): Suzuki, K. et al. Nature 540, 144-149, doi:10.1038/nature20565 (2016)).

In addition, in 2020, David Liu et al. reported an RNA-free mitochondrial cytosine base editor DdCBE (DddA-derived CBE), which achieved a major breakthrough in mitochondrial gene editing (Mok, B. Y. et al. Nature 583, 631 −+, doi:10.1038/s41586-020-2477-4 (2020))). Previously, due to the existence of mitochondrial double membrane, introducing sgRNA into mitochondria still faced great challenges, which severely limited the application of CRISPR/Cas9-based CBE tools in mitochondrial gene editing. Compared with CRISPR/Cas9-based CBE tools, the main changes of DdCBE include the following two points: one is to use TALE protein instead of sgRNA to realize the recognition of the target DNA strand, avoiding the difficulty that sgRNA is difficult to enter the mitochondria; the other is to use the newly discovered DddA, a double-stranded DNA deaminase, instead of APOBEC, to deaminate dC on the double-stranded DNA at the target site to dU, thereby realizing finally the base conversion from dC to dT.

In summary, there are a variety of cytosine base editing systems targeting the nucleus or mitochondria, and the list is still getting longer. But the core principle thereof is to deaminate cytosine (C) to uracil (U) at the target site; finally, these uracils are subjected to subsequent DNA replication process and converted from uracil (U) to thymine (T), thereby finally achieving the base conversion of C-to-T.

Since David Liu developed the cytosine base editor (Komor et al., 2016) in 2016, the adenine base editor (ABE) (Gaudelli et al., 2017) was also released in 2017. The main editing principle of this technology is: Cas9 reaches the target site under the guidance of sgRNA, opens the DNA double strand to form an R-loop structure, and then the adenine deaminase fused with Cas9) will deaminate the adenine in the editing window to form inosine (I). During repair and replication processes, inosine will be read as G by DNA polymerase, resulting in the eventual conversion from adenine (A) to guanine (G). After several years of development, the ABEmax system is currently used more frequently. Based on the original ABE version, this system has undergone a series of improvements such as mutation screening, codon optimization, and introduction of nuclear localization signals, which have continuously improved the editing efficiency of target sites. In 2020, David Liu and Jennifer A. Doudna reported a new version of ABE with higher activity and named it ABE8e (Richter et al., 2020). ABE8e retains only one TadA element on the basis of ABEmax, and has carried out multiple mutations, which not only improves the in vitro activity of enzyme (Lapinaite et al., 2020), but also significantly improves the editing efficiency of intracellular target sites.

Similarly, like the CBE editing system, a variety of ABE editing systems have been developed, and the core principle thereof is to deaminate adenine into inosine at the target site; then, these inosines undergo the subsequent DNA replication process to convert inosine to guanine, thereby finally realizing the base conversion of adenine (A) to guanine (G) (A-to-G).

In addition, in 2020, four research groups successively developed the adenine and cytosine dual base editing system (ACBE) (Grunewald et al., 2020; Li et al., 2020; Sakata et al., 2020; Zhang et al. al., 2020), the basic principle thereof is to combine the previously developed ABE and CBE technologies to achieve simultaneous editing of adenine and cytosine within the same target editing window.

Ideal gene editing tools should only edit the desired target site according to the design, but in fact, both ZFN/TALEN and CRISPR/Cas systems have been found to have off-target risks. The so-called off-target means that the gene editing tools used make unnecessary edits at non-target positions. Once an off-target event occurs, it may damage the gene sequence or chromosomal structure, disturb the genome stability and normal cell function, which may cause various serious side effects and even induce cancer. Therefore, off-target effect is a fatal shortcoming of gene editing technology for those applications that require high safety of gene editing effect (e.g., clinical treatment-related applications). If base editors are to be used in practice, their off-target effects must be thoroughly, comprehensively and accurately assessed in advance.

In theory, to detect the off-target effect of base editors, the simplest and most direct way is to directly detect single nucleotide mutations generated by base editors through whole genome sequencing (WGS). However, it is well known that WGS has many limitations for its application: first, there are many single nucleotide variations (SNVs) in the genome naturally, and the DNA replication process and the later high-throughput sequencing process will also produce a lot of random errors, both will cause genomic background that affects the accuracy of detection, so that WGS has an extremely low sensitivity in detecting single nucleotide mutations; second, when using high-throughput sequencing technology to perform WGS on the whole genome, the coverage of sequencing reads is very uneven, so that a huge amount of data is often required to obtain enough information to evaluate the whole genome. Therefore, conventional WGS cannot effectively detect the off-target effects of base editors at the whole genome level.

Another method is to firstly look for possible off-target sites through software prediction (e.g., Cas-OFFinder, etc.), or to select sites from the identification results of GUIDE-seq on the CRISPR/Cas9 nuclease system, where base editing tools may cause off-target editing, and then the exact editing frequency of these sites was obtained by targeted deep sequencing. The so-called GUIDE-seq is a technique that detects off-target sites by tracking the double-strand breaks (DSB) generated during the editing process of nuclease system, but this technique is not suitable for the gene editing technologies (e.g., various base editors) that hardly generate DSB. Although the method of predicting the position and then performing single-site in-depth detection can quickly know and compare the off-target risks of different base editing tools to a certain extent, the results are not based on comprehensive considerations at the whole genome level, and the conclusions obtained may vary widely due to the selected sites.

At present, there are two mainstream technologies for comprehensively assessing the off-target effects of base editing systems: one is the detection technology based on in vitro incubation, such as Digenome-seq; the other is the technology based on SNP detection, such as GOTI.

In 2017, Jin-Soo Kim' team from South Korea made some modifications for the CBE system based on the existing Digenome-seq technology in its laboratory, and realized the in vitro detection of genome-wide off-target effects of the system (Kim. D. et al. Nature biotechnology 35, 475-480, doi:10.1038/nbt.3852 (2017)). The detection principle thereof is as follows: firstly. UDG enzyme is used to treat the genomic DNA incubated with BE3ΔUGI (BE3 with the UGI part deleted), in order to generate a single-strand break (for CBE) at the position of dU, or endonuclease Endo V that recognizes dI is used to cleave the editing strand to create a nick (for ABE), so that it forms a DSB together with the single-strand break formed by nCas9 cleavage; then the editing site information is obtained by capturing characteristic reads in the subsequent high-throughput sequencing results.

In 2019, Yang Hui's team reported an off-target detection technology called GOTI (genome-wide off-target analysis by two-cell embryo injection) (Zuo, E. et al. Science 364, 289-292. doi:10.1126/science.aav9973 (2019)). The core of the technology lies in the two-cell embryo injection method, that is, at the two-cell stage of the mouse embryo, the gene editing system with a red fluorescent signal is injected into one of the cells, and after the embryo has developed to generate a sufficient number of cells, the entire embryo is digested into multiple single cells, and the edited and non-edited cell progenies were screened out by flow cytometry. Theoretically, red fluorescent positive cells and negative cells are both from the same fertilized egg, so they should have the same genomic background, and the difference caused by gene editing can be obtained by comparing the two groups of cells through whole genome sequencing (WGS), thereby obtaining off-target information.

As far as the existing genome-wide detection technologies are concerned. Digenome-seq is an in vitro detection technology, and the off-target editing behavior will theoretically be affected by the real chromatin state and local protein concentration in living cells, so this technology cannot effectively reflect the real off-target situation in the in vivo environment. On the other hand, although GOTI and other technologies adopt the two-cell embryo injection strategy to eliminate the influence of genomic background such as SNV as much as possible, they still cannot avoid the DNA replication error background caused by single-cell amplification, and this method involves embryo manipulation, so that it has not a wide applicability, is technically difficult and time-consuming. In addition, this method still relies on whole-genome sequencing analysis, inevitably requires high sequencing costs to achieve sufficient data coverage for all embryo samples involved in the experiment, and thus is not suitable for high-throughput screening evaluation. More importantly, the conclusions of the two methods on the DNA off-target effect of base editing tools are almost completely contradictory. For example, Kim's team found that CBE has high specificity and will only cause a limited number of Cas-dependent off-targets, while Yang Hui's team only identified a large number of non-Cas-dependent off-targets. As well known in the art, the understanding of off-target effects largely determines the direction of subsequent optimization of base editors. For the art, it is clear that there is a need for a better, comprehensive off-target detection technology without detection bias.

Therefore, it is urgent to develop a sensitive, non-biased and economical novel detection technology for comprehensive evaluation of off-target effects of base editing systems at the genome-wide level.

### Contents of the Present Invention

Based on in-depth research, the inventors of the present application have developed a new method capable of detecting the editing site, editing efficiency or off-target effect of a base editor (e.g., a single base editor or a dual base editor) editing a nucleic acid. The method of the present application can capture a base editing intermediate produced in a living cell by various base editors (e.g., a single base editor or a dual base editor) during the editing process, and effectively mark and enrich the editing site. Thus, the method of the present application can be generally applied to the detection of editing sites of various base editing tools, can evaluate their editing efficiency or off-target effect, and can achieve high-sensitivity detection at the genome-wide level.

Therefore, in one aspect, the present application provides a method for detecting an editing site, editing efficiency or off-target effect of a base editor (e.g., a single base editor or a dual base editor) editing a target nucleic acid, which comprises the following steps:
(1) providing an edit product of a base editor editing a target nucleic acid, in which the edit product comprises a base editing intermediate, and the base editing intermediate comprises a first nucleic acid strand and a second nucleic acid strand; wherein, the first nucleic acid strand comprises an edited base generated by the base editor editing the target nucleic acid;
(2) generating a single-strand break in a segment comprising the edited base (e.g., in a segment from 10 nt upstream to 10 nt downstream of the edited base) in the first nucleic acid strand:
(3) introducing a nucleotide labeled with a first labeling molecule at or downstream of the single-strand break, to produce a labeled product comprising the first labeling molecule;
(4) isolating or enriching the labeled product; for example, isolating or enriching the labeled product by using a first binding molecule capable of specifically recognizing and binding the first labeling molecule;
(5) sequencing the labeled product;

thereby, determining the editing site, editing efficiency or off-target effect of the base editor editing the target nucleic acid.

The method of the present application can be used to detect the editing site, editing efficiency or off-target effect of various base editors editing a target nucleic acid. In some preferred embodiments, the base editor is a single base editor or a dual base editor. In some preferred embodiments, the base editor is selected from the group consisting of cytosine single base editor, adenine single base editor, and adenine and cytosine dual base editor.

The method of the present application is not limited by the target nucleic acid being edited. In certain preferred embodiments, the target nucleic acid is a genomic nucleic acid. In certain preferred embodiments, the target nucleic acid is a mitochondrial nucleic acid.

In some preferred embodiments, the edit product in step (1) is a product of the base editor editing the target nucleic acid outside a cell, inside a cell, or inside an organelle (e.g., a nucleus or a mitochondria).

In some preferred embodiments, the method further comprises the following step before step (1): contacting the base editor with the target nucleic acid under a condition that allows the base editor to edit the target nucleic acid, thereby generating the edit product. The condition that allows the base editor to edit the target nucleic acid may be any condition suitable for the base editor used to exert its editing activity.

In certain preferred embodiments, under the condition that allows the base editor to edit the target nucleic acid, the base editor is contacted with the target nucleic acid outside a cell, inside a cell, or inside an organelle (e.g., a nucleus or a mitochondria) to produce the edit product.

For example, the method further comprises the following step before step (1): introducing the base editor into a cell or organelle, so that the base editor is contacted with the target nucleic acid in the cell or organelle and performs base editing, thereby generating the edit product; or, introducing a nucleic acid molecule encoding the base editor into a cell or organelle and allowing it to express the base editor, so that the base editor is contacted with the target nucleic acid in the cell or organelle and performs base editing, thereby generating the edit product.

In some preferred embodiments, in step (1), the target nucleic acid underwent the base editing is extracted or isolated from the cell or organelle, and optionally, subjected to fragmentation, so as to obtain the edit product.

The fragmentation can be carried out by any means suitable for nucleic acid fragmentation, such as by ultrasonication or random enzymatic digestion. In certain embodiments, where fragmentation is performed, the edit product may be a nucleic acid fragment with or without an overhanging end. In certain preferred embodiments, the fragmentation (e.g., fragmentation using an endonuclease) results in a nucleic acid fragment comprising an overhanging end (e.g., cohesive end). In such embodiments, the nucleic acid fragment comprising overhanging end is optionally subjected to end repair, so as to produce a nucleic acid fragment with blunt end, which can be used as the edit product for the next step. For example, the end repair can comprise the filling-in of 5′ end overhang (e.g., by nucleic acid polymerization) and/or the excision of 3′ end overhang. In certain preferred embodiments, the end repair comprises the filling-in of 5′ end overhang (e.g., by nucleic acid polymerization).

In some preferred embodiments, the second nucleic acid strand does not undergo base editing or does not comprise an edited base.

However, it is easy to understand that due to the existence of off-target situations, the base editor may perform base editing at multiple editing sites (including on-target sites and off-target sites). For example, the base editor may edit both nucleic acid strands of genomic DNA or organelle DNA (e.g., mitochondrial DNA). Therefore, in some cases, the second nucleic acid strand potentially undergoes base editing and may comprise an edited base. Thus, in certain embodiments, the second nucleic acid strand undergoes base editing and/or comprises an edited base.

In certain preferred embodiments, the edited base is selected from the group consisting of uracil or inosine.

In some preferred embodiments, in step (2), the single-strand break is generated at the position of the edited base or its upstream (e.g., within 10 nt, within 9 nt, within 8 nt, within 7 nt, within 6 nt, within 5 nt, within 4 nt, within 3 nt, within 2 nt, within 1 nt upstream) or downstream (e.g., within 10 nt, within 9 nt, within 8 nt, within 7 nt, within 6 nt, within 5 nt, within 4 nt, within 3 nt, within 2 nt, within 1 nt downstream).

In some preferred embodiments, before performing step (2), the method further comprises: a step of repairing a possible single-strand break (SSB) (e.g., endogenous single-strand break) in the edit product. For example, before performing step (2), the method further comprises: using a nucleic acid polymerase, a nucleotide (e.g., nucleotide without label; such as dNTP without label) and a nucleic acid ligase (e.g., DNA ligase) to repair a possible SSB (e.g., endogenous SSB) in the edit product.

For example, before performing step (2), the method further comprises: (i) incubating the edit product with a nucleic acid polymerase (e.g., DNA polymerase) and a nucleotide molecule (preferably, dNTP without label) under a condition allowing nucleic acid polymerization; and, (ii) ligating a nick in the product of step (i) using a nucleic acid ligase (e.g., DNA ligase). In certain preferred embodiments, the nucleic acid polymerase (e.g., DNA polymerase) has strand displacement activity.

Without being bound by theory, it is advantageous to perform the repair of SSB before step (2). For example, the repair of SSB can eliminate a break possibly existing in the edit product. including endogenous SSB, and SSB possibly introduced during nucleic acid manipulation (e.g., nucleic acid fragmentation). Thus, the introduction of a nucleotide labeled with a first labeling molecule at or downstream of these pre-existing SSBs in subsequent steps can be avoided, and thus the interference of these pre-existing SSBs on the detection results can be avoided.

In certain preferred embodiments, in step (2), an endonuclease (e.g., endonuclease V, endonuclease VIII or AP endonuclease) is used to generate a single-strand break in the first nucleic acid strand.

In some preferred embodiments, the nucleotide labeled with the first labeling molecule is selected from the group consisting of uracil deoxyribonucleotide labeled with the first labeling molecule (e.g., dUTP labeled with the first labeling molecule), cytosine deoxyribonucleotide labeled with the first labeling molecule (e.g., dCTP labeled with the first labeling molecule), thymidine deoxyribonucleotide labeled with the first labeling molecule (e.g., dTTP labeled with the first labeling molecule), adenine deoxyribonucleotide labeled with the first labeling molecule (e.g., dATP labeled with the first labeling molecule), guanine deoxyribonucleotide labeled with the first labeling molecule (e.g., dGTP labeled with the first labeling molecule), or any combination thereof.

In certain preferred embodiments, the nucleotide labeled with the first labeling molecule is uracil deoxyribonucleotide labeled with the first labeling molecule (e.g., dUTP labeled with the first labeling molecule) or guanine deoxyribonucleotide labeled with the first labeling molecule (e.g., dGTP labeled with the first labeling molecule).

In certain preferred embodiments, the first labeling molecule and the first binding molecule constitute a molecular pair capable of specific interaction (e.g., capable of specifically binding to each other). Such molecular pairs capable of specific interaction (e.g., capable of specifically binding to each other) are well known to those skilled in the art, for example, biotin or functional variant thereof—avidin or functional variant thereof (e.g., biotin-avidin, biotin-streptavidin). antigen/hapten—antibody, enzyme and cofactor, receptor-ligand, molecular pairs capable of click chemistry (e.g., alkynyl-comprising group azido-comprising compound), etc. In certain preferred embodiments, the first labeling molecule is biotin or a functional variant thereof, and the first binding molecule is avidin or a functional variant thereof; or, the first labeling molecule is a hapten or antigen, and the first binding molecule is an antibody specific for the hapten or antigen; or, the first labeling molecule is an alkynyl-comprising group (e.g., an ethynyl), and the first binding molecule is an azido-comprising compound that can undergo a click chemical reaction with the alkynyl (e.g., ethynyl). For example, the nucleotide labeled with the first labeling molecule is a nucleotide comprising an ethynyl (e.g., 5-ethynyl-dUTP), and the first binding molecule is an azido-comprising compound (e.g., azide magenetic beads) capable of performing a click chemical reaction with the ethynyl.

In certain preferred embodiments, in the nucleotide labeled with the first labeling molecule, the first labeling molecule is reversibly or irreversibly ligated to the nucleotide.

In some preferred embodiments, in the nucleotide labeled with the first labeling molecule, the first labeling molecule is reversibly ligated to the nucleotide. In such embodiments, after performing step (4), the method may further comprise a step of removing the first labeling molecule from the labeled product. In some cases, the removal of the first labeling molecule is advantageous, for example, its adverse effects on subsequent amplification and/or sequencing steps can be avoided.

In some preferred embodiments, in the nucleotide labeled with the first labeling molecule, the first labeling molecule is irreversibly ligated to the nucleotide. In such embodiments, preferably, the presence of the first labeling molecule does not adversely affect the amplification and/or sequencing of the labeled product. For example, in some preferred embodiments, the labeled product produced in step (3) can be subjected to nucleic acid amplification reaction. For example, the labeled product can be subjected to a nucleic acid amplification reaction with a nucleic acid polymerase (e.g., a high-fidelity or low-fidelity nucleic acid polymerase).

In certain preferred embodiments, the nucleotide labeled with the first labeling molecule is introduced into the single-strand break or downstream thereof by nucleic acid polymerization, thereby producing a labeled product comprising the first labeling molecule. For example, in step (3), a nucleic acid polymerase (e.g., a nucleic acid polymerase having strand displacement activity) is used to introduce the nucleotide labeled with the first labeling molecule into the single-strand break or downstream thereof. For example, in step (3), the first nucleic acid strand is incubated with a nucleic acid polymerase and the nucleotide labeled with the first labeling molecule under a condition that allows nucleic acid polymerization; wherein the nucleic acid polymerase initiates an extension reaction at the single-strand break by using the second nucleic acid strand as a template, and incorporates the nucleotide labeled with the first labeling molecule into the single-strand break or downstream thereof.

In some preferred embodiments, in step (3), the method further comprises a step of using a nucleic acid ligase (e.g., DNA ligase) to ligate a nick in the labeled product comprising the first labeling molecule.

In some preferred embodiments, in step (3), a nucleotide labeled with a second labeling molecule is also introduced at or downstream of the single-strand break, thereby generating a labeled product comprising the first labeling molecule and the second labeling molecule.

In certain preferred embodiments, the nucleotide labeled with the second labeling molecule is a nucleotide molecule that is capable of complementary base pairing with different nucleotides under different conditions (e.g., before and after undergoing a treatment). For example, the nucleotide labeled with the second labeling molecule is capable of complementary base pairing with a first nucleotide before undergoing a treatment, and capable of complementary base pairing with a second nucleotide after undergoing a treatment.

In some preferred embodiments, the nucleotide labeled with the second labeling molecule is selected from the group consisting of d5fC (5-formylcytosine deoxyribonucleotide), d5caC (5-carboxycytosine deoxyribonucleotide), d5hmC (5-hydroxymethylcytosine deoxyribonucleotide), and dac⁴C (N4-acetylcytosine deoxyribonucleotide).

In certain preferred embodiments, the nucleotide labeled with the second labeling molecule is a modified cytosine deoxyribonucleotide capable of complementary base pairing with a first nucleotide (e.g., guanine deoxyribonucleotide) before undergoing a treatment, and capable of complementary base pairing with a second nucleotide (e.g., adenine deoxyribonucleotide) after undergoing a treatment. In some preferred embodiments, the nucleotide labeled with the second labeling molecule is selected from the group consisting of d5fC (5-formylcytosine deoxyribonucleotide), d5caC (5-carboxycytosine deoxyribonucleotide), d5hmC (5-hydroxymethylcytosine deoxyribonucleotide) and dac⁴C (N4-acetylcytosine deoxyribonucleotide).

For example, the nucleotide labeled with the second labeling molecule is 5-formylcytosine deoxyribonucleotide. 5-Formylcytosine deoxyribonucleotide is capable of complementary base pairing with guanine deoxyribonucleotide before the treatment with a compound (e.g., malononitrile, borane (e.g., pyridine borane compound, such as pyridine borane or 2-picoline borane), or azido-indandione), whereas capable of complementary base pairing with adenine deoxyribonucleotide after the treatment with a compound (e.g., malononitrile, borane (e.g., pyridine borane compound, such as pyridine borane or 2-picoline borane), or azido-indandione) (see, for example, Liu, Y. et al. Bisulfite-free direct detection of 5-methylcytosine and 5-hydroxymethylcytosine at base resolution. Nature biotechnology 37, 424-429, doi:10.1038/s41587-019-0041-2 (2019).; patent document WO2015043493A1, these references are incorporated herein by reference in their entirety).

For example, the nucleotide labeled with the second labeling molecule is 5-carboxycytosine deoxyribonucleotide. 5-Carboxycytosine deoxyribonucleotide is capable of complementary base pairing with guanine deoxyribonucleotide before the treatment with a compound (e.g., pyridine borane compound (e.g., pyridine borane or 2-picoline borane)), whereas capable of complementary base pairing with adenine after the treatment with a compound (e.g., pyridine borane compound (e.g., pyridine borane or 2-picoline borane)) (see, for example, Liu, Y. et al. Bisulfite-free direct detection of 5-methylcytosine and 5-hydroxymethylcytosine at base resolution. Nature biotechnology 37, 424-429, doi:10.1038/s41587-019-0041-2 (2019)., which is hereby incorporated by reference in its entirety).

For example, the nucleotide labeled with the second labeling molecule is 5-hydroxymethylcytosine deoxyribonucleotide. 5-Hydroxymethylcytosine deoxyribonucleotide can be converted into 5-formylcytosine deoxyribonucleotide under the catalysis of an oxidant (e.g., potassium ruthenate) or oxidase (e.g., TET (ten-eleven translocation) protein), while 5-formylcytosine deoxyribonucleotide is capable of complementary base pairing with guanine deoxyribonucleotide before the treatment with a compound (e.g., malononitrile, borane compound (e.g., pyridine borane compound, such as pyridine borane or 2-picoline borane), or azido-indandione), whereas capable of complementary base pairing with adenine deoxyribonucleotide after the treatment with a compound (e.g., malononitrile, borane compound (e.g., pyridine borane compound, such as pyridine borane or 2-picoline borane), or azido-indandione).

For example, the nucleotide labeled with the second labeling molecule is N4-acetylcytosine deoxyribonucleotide (dac⁴C). N4-Acetylcytosine deoxyribonucleotide is capable of complementary base pairing with guanine deoxyribonucleotide before the treatment with a compound (e.g., sodium cyanoborohydride), whereas capable of complementary base pairing with adenine deoxyribonucleotide after the treatment with a compound (e.g., sodium cyanoborohydride) (see, for example, Nature 583, 638-643 (2020), DOI: 10.1038/s41586-020-2418-2, which is hereby incorporated by reference in its entirety).

In some preferred embodiments, the nucleotide labeled with the first labeling molecule and the nucleotide labeled with the second labeling molecule are introduced at the single-strand break or downstream thereof, thereby producing a labeled product comprising the first labeling molecule and the second labeling molecule. For example, in step (3), the first nucleic acid strand is incubated with a nucleic acid polymerase (e.g., a nucleic acid polymerase having strand displacement activity) and the nucleotide labeled with the first labeling molecule and the nucleotide labeled with the second labeling molecule under a condition allowing nucleic acid polymerization; wherein, the nucleic acid polymerase initiates an extension reaction using the second nucleic acid strand as a template at the single-strand break, and incorporating the nucleotide labeled with the first labeling molecule and the nucleotide labeled with the second labeling molecule at or downstream of the single strand break. In some preferred embodiments, in step (3), the method further comprises a step of using a ligase to ligate a nick in the labeled product comprising the first labeling molecule and the second labeling molecule.

It can be understood that the nucleotide labeled with the first labeling molecule and the nucleotide labeled with the second labeling molecule can be introduced in the same nucleic acid polymerization reaction, or can be introduced in different nucleic acid polymerization reactions, as long as the labeled product comprising the first labeling molecule and the second labeling molecule can be produced.

In certain embodiments, the use or incorporation of the nucleotide labeled with the second labeling molecule is advantageous. It is easy to understand that the nucleotide labeled with the second labeling molecule can be incorporated into the labeled product by way of complementary base pairing through nucleic acid polymerization. In this case, the nucleotide labeled with the second labeling molecule (e.g., 5-formylcytosine deoxyribonucleotide) is incorporated into the labeled product through the complementary pairing capability with a first base (e.g., guanine deoxyribonucleotide). Subsequently, the labeled product can be treated (e.g., treated with a compound such as malononitrile, borane compound (e.g., pyridine borane, such as pyridine borane or 2-picoline borane), or azido-indandione), whereby the nucleotide labeled with the second labeling molecule in the labeled product will be modified or changed, and undergoes complementary base pairing with a second base (e.g., adenine deoxyribonucleotide). Therefore, when the labeled product as treated is sequenced, the nucleotide at the incorporation position of the nucleotide labeled by the second labeling molecule will pair with the second base and be read as a complementary base of the second base (rather than complementary base of the first base) in the sequencing result. In other words, in the sequencing result of the labeled product as treated, a base mutation signal that the complementary base of the first base is mutated to the complementary base of the second base (e.g., C-to-T mutation signal) will be generated at the position where the nucleotide labeled with the second labeling molecule is incorporated. By detecting the base mutation signal, the incorporation position of the nucleotide labeled by the second labeling molecule can be determined, and then the edited base adjacent thereto can be accurately positioned. In addition, one or more nucleotides labeled with the second labeling molecule can be incorporated into the labeled product by nucleic acid polymerization, whereby one or more base mutation signals will be detected in the sequencing results of the labeled product as treated. This can amplify the base mutation signal and improve the sensitivity of detection.

Thus, in the embodiments of using the nucleotide labeled with the second labeling molecule, preferably after step (3), the labeled product is treated to alter the complementary base pairing capability of the nucleotide labeled with the second labeling molecule comprised therein.

In certain preferred embodiments, the nucleotide labeled with the second labeling molecule is a modified cytosine deoxyribonucleotide. In such embodiments, after step (3), the labeled product is treated to alter the complementary base pairing capability of the modified cytosine deoxyribonucleotide comprised therein (e.g., allowing it to pair with adenine deoxyribonucleotide, rather than pairing with guanine deoxyribonucleotide).

In certain preferred embodiments, the nucleotide labeled with the second labeling molecule is 5-formylcytosine deoxyribonucleotide. In such embodiments, after step (3), the labeled product is treated with a compound (e.g., malononitrile, borane compound (e.g., pyridine borane compound, such as pyridine borane or 2-picoline borane), or azido-indandione) to change the complementary base-pairing capability of the 5-formylcytosine deoxyribonucleotide comprised therein.

In certain preferred embodiments, the nucleotide labeled with the second labeling molecule is 5-carboxycytosine deoxyribonucleotide. In such embodiments, after step (3), the labeled product is treated with a compound (e.g., borane compound, (e.g., pyridine borane comopund. such as pyridine borane or 2-picoline borane) to change the complementary base pairing capability of the 5-carboxycytosine deoxyribonucleotide comprised therein.

In certain preferred embodiments, the nucleotide labeled with the second labeling molecule is 5-hydroxymethylcytosine deoxyribonucleotide. In such embodiments, after step (3), the labeled product is first treated with an oxidant (e.g., potassium ruthenate) or an oxidase (e.g., TET protein), and then treated with a compound (e.g., malononitrile, borane compound (e.g., pyridine borane compound, such as pyridine borane or 2-picoline borane), or azido-indandione) to change the complementary base pairing capability of the 5-hydroxymethylcytosine deoxyribonucleotide comprised therein.

In some preferred embodiments, the nucleotide labeled with the second labeling molecule is N4-acetylcytosine deoxyribonucleotide (dac⁴C). In such embodiments, after step (3), the labeled product is treated with a compound (e.g., sodium cyanoborohydride) to alter the complementarity base pairing capability of the N4-acetylcytosine deoxyribonucleotide comprised therein.

Preferably, the step of treating the labeled product is performed before sequencing the labeled product, for example, before step (4) or before step (5).

In some cases, the nucleotide labeled with the second labeling molecule (e.g., 5-formylcytosine deoxyribonucleotide, 5-hydroxymethylcytosine deoxyribonucleotide) may be a nucleotide naturally occurring in cells. To avoid adverse effects of such naturally occurring nucleotide labeled with the second labeling molecule (e.g., resulting in false positive signals), the nucleotide labeled with the second labeling molecule that may be present in the edit product can be protected (e.g., endogenous 5-formylcytosine deoxyribonucleotide can be protected using ethylhydroxylamine, or, endogenous 5-hydroxymethylcytosine deoxyribonucleotide can be protected using the glycosylation reaction catalyzed by β-glucosyltransferase (βGT)) before step (3) (e.g., before step (2)) to prevent a change in its complementary base pairing capability.

Thus, in certain embodiments using the nucleotide labeled with the second labeling molecule (e.g., 5-formylcytosine deoxyribonucleotide, 5-hydroxymethylcytosine deoxyribonucleotide), the nucleotide labeled with the second labeling molecule that may be present in the edit product is protected before step (3) (e.g., before step (2)).

For example, in certain embodiments, the nucleotide labeled with the second labeling molecule is 5-formylcytosine deoxyribonucleotide. In such embodiments, preferably, the endogenous 5-formylcytosine deoxyribonucleotide is protected with ethylhydroxylamine before step (3) (e.g., before step (2)).

For example, in certain embodiments, the nucleotide labeled with the second labeling molecule is 5-hydroxymethylcytosine deoxyribonucleotide. In such embodiments, preferably, before step (3) (e.g., before step (2)), endogenous 5-hydroxymethylcytosine deoxyribonucleotide is protected by βGT-catalyzed glycosylation reaction (see, Cell, 18 Apr. 2013, 153(3): 678-691, DOI: 10.1016/j.cell.2013.04.001, which is incorporated herein by reference in its entirety).

In some cases, the nucleotide labeled with the second labeling molecule (e.g., 5-carboxycytosine deoxyribonucleotide, N4-acetylcytosine deoxyribonucleotide) is not a nucleotide naturally occurring in cells, or is a nucleotide naturally occurring in cells in a very small amount. In this case, there is no need to carry out the nucleotide protection for the edit product before step (3).

Thus, in certain embodiments using the nucleotide labeled with the second labeling molecule (e.g., 5-carboxycytosine deoxyribonucleotide, N4-acetylcytosine deoxyribonucleotide), the edit product does not undergo nucleotide protection before step (3).

In some preferred embodiments, in step (2), a single-strand break is generated at the position of the edited base; and, in step (3), the nucleotide labeled with the first labeling molecule and the nucleotide labeled with the second labeling molecule are introduced at or downstream of the position of the single-strand break, thereby producing a labeled product comprising the first labeling molecule and the second labeling molecule.

In certain preferred embodiments, in step (2), a single-strand break is generated downstream of the edited base; and, in step (3), at or downstream of the single-strand break, the nucleotide labeled with the first labeling molecule is introduced, and optionally, the nucleotide labeled with the second labeling molecule is introduced, thereby producing a labeled product comprising the first labeling molecule and optionally the second labeling molecule.

In certain preferred embodiments, in step (4), the labeled product is isolated or enriched using a first binding molecule attached to a solid support. Various suitable solid supports can be used to support the first binding molecule. For example, the solid support can be selected from the group consisting of magnetic beads, agarose beads, or chips.

In some preferred embodiments, before performing step (5), the method further comprises: amplifying the labeled product as isolated or enriched in step (4); and/or, constructing a sequencing library with the labeled product as isolated or enriched in step (4).

In some preferred embodiments, in step (4), the labeled product as isolated or enriched comprises a nucleic acid single strand comprising the nucleotide labeled with the first labeling molecule and/or the nucleotide labeled with the second labeling molecule. For example, in certain embodiments, the labeled product can be subjected to a melting treatment (e.g., alkali treatment), and then, the first binding molecule capable of specifically recognizing and binding the first labeling molecule is used to isolate or enrich a nucleic acid single strand comprising the nucleotide labeled with the first labeling molecule and/or the nucleotide labeled with the second labeling molecule in the labeled product. In certain embodiments, the labeled product can be isolated or enriched using a first binding molecule capable of specifically recognizing and binding to the first labeling molecule, and then the labeled product is subjected to a melting treatment (e.g., alkali treatment), so as to obtain a nucleic acid single strand comprising the nucleotide labeled with the first labeling molecule and/or the nucleotide labeled with the second labeling molecule in the labeled product. In some preferred embodiments, the melting treatment (e.g., alkali treatment) is carried out in a condition under which the binding between the first labeling molecule and the first binding molecule is remained.

In some preferred embodiments, before performing step (5), the labeled product as isolated or enriched in step (4) is amplified using a nucleic acid polymerase (e.g., a low-fidelity nucleic acid polymerase and/or a high-fidelity nucleic acid polymerase). For example, in certain preferred embodiments, the step of amplifying comprises:

performing up to 5 (e.g., up to 1, up to 2, up to 3, up to 4, up to 5) cycles of polymerase chain reaction using a low-fidelity nucleic acid polymerase; and,

performing at least 3 (e.g., at least 3, at least 5, at least 10, at least 20, at least 30, at least 40) cycles of polymerase chain reaction using a high-fidelity nucleic acid polymerase.

It can be understood that various suitable methods can be used to construct a sequencing library from the labeled product as isolated or enriched in step (4). Such methods of constructing the sequencing library are not limited. For example, according to the sequencing method used, a sequencing library with corresponding characteristics can be constructed. For example, according to the needs of sequencing, corresponding sequencing or amplification oligonucleotide adapters can be added to the ends of the labeled product. In certain embodiments, a dA tail can be added to the 3′ end of the labeled product, which can be used for ligation to an oligonucleotide adapter comprising a dT tail.

In some preferred embodiments, in step (5), the sequence of the labeled product is determined by sequencing (e.g., second-generation sequencing or third-generation sequencing), hybridization or mass spectrometry.

In some preferred embodiments, the method further comprises comparing the sequence determined in step (5) with a reference sequence, so as to determine the editing site, editing efficiency or off-target effect of the base editor editing the target nucleic acid.

In some preferred embodiments, the reference sequence is the target nucleic acid sequence before base editing. For example, the target nucleic acid sequence before base editing can be obtained from a database, or can be obtained by a sequencing method.

### Cytosine Base Editor and Evaluation Thereof

In a preferred embodiment, the base editor is a cytosine base editor (e.g., a nuclear cytosine base editor, an organelle cytosine base editor). In certain preferred embodiments, the cytosine base editor is a cytosine base editor capable of editing cytosine into uracil. For a detailed description of cytosine base editors, see, for example, Andrew V. Anzalone, et al. Nature biotechnology 38(7), 824-844, doi: 10.1038/s41587-020-0561-9 (2020), which is incorporated herein by reference in its entirety. In certain preferred embodiments, the base editor is a cytosine base editor capable of editing a nuclear nucleic acid or a cytosine base editor capable of editing a mitochondrial nucleic acid.

In certain preferred embodiments, the edited base is uracil.

In certain preferred embodiments, the base editing intermediate is a uracil-comprising nucleic acid molecule (e.g., a DNA molecule).

In certain preferred embodiments, the nucleotide labeled with the second labeling molecule is a modified cytosine deoxyribonucleotide capable of undergoing complementary base pairing with a first nucleotide (e.g., guanine deoxyribonucleotide) before a treatment, and capable of undergoing complementary base pairing with a second nucleotide (e.g., adenine deoxyribonucleotide) after a treatment. In some preferred embodiments, the nucleotide labeled with the second labeling molecule is selected from the group consisting of d5fC (5-formylcytosine deoxyribonucleotide), d5caC (5-carboxycytosine deoxyribonucleotide), d5hmC (5-hydroxymethylcytosine deoxyribonucleotide) and dac⁴C (N4-acetylcytosine deoxyribonucleotide).

In some preferred embodiments, in step (2), an AP site-specific endonuclease (e.g., AP endonuclease) is used to generate a single-strand break at the position of the edited base in the first nucleic acid strand; and, in step (3), the nucleotide labeled with the first labeling molecule and the nucleotide labeled with the second labeling molecule are introduced at or downstream of the single-strand break to produce a labeled product comprising the first labeling molecule and the second labeling molecule. Subsequently, step (4) to step (5) may be carried out as described above, thereby determining the editing site, editing efficiency or off-target effect of the cytosine base editor editing the target nucleic acid.

In some preferred embodiments, before step (2), the method further comprises a step of forming an AP site at the position of the edited base in the first nucleic acid strand.

For example, in some preferred embodiments, before step (2), the method further comprises: a step of incubating the edit product with UDG (uracil-DNA glycosylase). UDG can specifically recognize uracil nucleotide in the nucleic acid chain, and can specifically excise the uracil on the nucleotide, thereby forming an AP site (apurinic/apyrimidinic site) in the nucleic acid chain. Thus, incubation of UDG with the edit product is able to convert the edited base (uracil) in the first nucleic acid strand into an AP site.

In some preferred embodiments, before the step of incubating with UDG, the method further comprises a step of repairing an AP site possibly existing in the edit product.

In some preferred embodiments, the step of repairing AP site comprises:
(a) incubating an AP endonuclease with the edit product in which an AP site is possibly present under a condition that allows the AP endonuclease to exert cleavage activity thereof;
(b) incubating the product of step (a) with a nucleic acid polymerase (e.g., DNA polymerase) and a nucleotide molecule (e.g., a nucleotide molecule that is not labeled with the first labeling molecule or the second labeling molecule; for example, dNTP without label) under a condition that allows nucleic acid polymerization;
(c) incubating the product of step (b) with a nucleic acid ligase (e.g., DNA ligase) under a condition that allows the nucleic acid ligase to exert ligation activity thereof,
thereby, repairing the AP site possibly existing in the edit product.

It is easy to understand that in step (a), the AP endonuclease can cause the edit product to generate a single-strand break at the possible AP site. In step (b), the nucleic acid polymerase can initiate an extension reaction at the single-strand break using the second nucleic acid strand as a template, and repair the single-strand break generated in step (a). In step (c), the nucleic acid ligase (e.g., DNA ligase) is capable of ligating a nick in the product of step (b). In certain preferred embodiments, the nucleic acid polymerase (e.g., DNA polymerase) in step (b) has strand displacement activity.

Without being bound by theory, it is advantageous to perform AP site repair before step (2). For example, the AP site repair can eliminate AP sites that may be present in the edit product. Thereby, the introduction of the nucleotide labeled with the first labeling molecule and the nucleotide labeled with the second labeling molecule at or downstream of these pre-existing AP sites in subsequent steps can be avoided, and the interference on detection results by these pre-existing AP sites can be avoided.

In certain preferred embodiments, after step (3), the labeled product is treated to change the complementary base pairing capability of the nucleotide labeled with the second labeling molecule comprised therein. In certain preferred embodiments, the nucleotide labeled with the second labeling molecule is a modified cytosine deoxyribonucleotide. In such embodiments, after step (3), the labeled product is treated to alter the complementary base pairing capability of the modified cytosine deoxyribonucleotide comprised therein (e.g., allow it to pair with adenine deoxyribonucleotide, instead of guanine deoxyribonucleotide).

In certain preferred embodiments, the nucleotide labeled with the second labeling molecule is 5-formylcytosine deoxyribonucleotide. In such embodiments, after step (3), the labeled product is treated with a compound (e.g., malononitrile, borane compound (e.g., pyridine borane compound, such as pyridine borane or 2-picoline borane), or azido-indandione) to change the complementary base pairing capability of the 5-formylcytosine deoxyribonucleotide comprised therein.

In certain preferred embodiments, the nucleotide labeled with the second labeling molecule is 5-carboxycytosine deoxyribonucleotide. In such embodiments, after step (3), the labeled product is treated with a compound (e.g., borane compound (e.g., pyridine borane compound, such as pyridine borane or 2-picoline borane)) to change the complementary base pairing capability of the 5-carboxycytosine deoxyribonucleotide comprised therein.

In certain preferred embodiments, the nucleotide labeled with the second labeling molecule is 5-hydroxymethylcytosine deoxyribonucleotide. In such embodiments, after step (3), the labeled product is first treated with an oxidant (e.g., potassium ruthenate) or an oxidase (e.g., TET protein), and then treated with a compound (e.g., malononitrile, borane compound (e.g., pyridine borane compound, such as pyridine borane or 2-picoline borane), or azido-indandione) to change the complementary base pairing capability of the 5-hydroxymethylcytosine deoxyribonucleotide comprised therein.

In some preferred embodiments, the nucleotide labeled with the second labeling molecule is N4-acetylcytosine deoxyribonucleotide (dac⁴C). In such embodiments, following step (3), the labeled product is treated with a compound (e.g., sodium cyanoborohydride) to alter the complementarity base pairing capability of the N4-acetylcytosine deoxyribonucleotide comprised therein.

Preferably, the step of treating the labeled product is performed before sequencing the labeled product, for example, before step (4) or before step (5).

In some embodiments, before step (3) (e.g., before step (2)), the nucleotide labeled with the second labeling molecule that may possibly be present in the edit product is protected. For example, before step (3) (e.g., before step (2)), endogenous 5-formylcytosine deoxyribonucleotide can be protected using ethylhydroxylamine, or, endogenous 5-hydroxymethylcytosine deoxyribonucleotide can be protected by the βGT-catalyzed glycosylation reaction.

For example, in certain embodiments using the nucleotide labeled with the second labeling molecule (e.g., 5-formylcytosine deoxyribonucleotide, 5-hydroxymethylcytosine deoxyribonucleotide), before step (3) (e.g., before step (2)), the nucleotide labeled with the second labeling molecule that may possibly exist in the edit product is protected.

For example, in certain embodiments, the nucleotide labeled with the second labeling molecule is 5-formylcytosine deoxyribonucleotide. In such embodiments, preferably, the endogenous 5-formylcytosine deoxyribonucleotide is protected with ethylhydroxylamine before step (3) (e.g., before step (2)).

For example, in certain embodiments, the nucleotide labeled with the second labeling molecule is 5-hydroxymethylcytosine deoxyribonucleotide. In such embodiments, preferably, before step (3) (e.g., before step (2)), endogenous 5-hydroxymethylcytosine deoxyribonucleotide is protected by βGT-catalyzed glycosylation reaction.

In certain embodiments using the nucleotide labeled with the second labeling molecule (e.g., 5-carboxycytosine deoxyribonucleotide, N4-acetylcytosine deoxyribonucleotide), the edit product does not undergo nucleotide protection before step (3).

### Adenine Base Editor and Evaluation Thereof

In a preferred embodiment, the base editor is an adenine base editor. In some preferred embodiments, the adenine base editor is an adenine base editor capable of editing adenine into inosine, such as adenine base editors ABE7.10, ABEmax, and ABE8e. A detailed description of adenine base editors can be found, for example, in Andrew V. Anzalone, et al. Nature biotechnology 38(7), 824-844, doi: 10.1038/s41587-020-0561-9 (2020), which is incorporated herein by reference in its entirety.

In some preferred embodiments, the edited base is inosine.

In some preferred embodiments, the base editing intermediate is a nucleic acid molecule (e.g., a DNA molecule) comprising inosine.

In some preferred embodiments, in step (2), a inosine site-specific endonuclease (e.g., endonuclease V, or endonuclease VIII) is used to generate a single-strand break at or downstream of the edited base in the first nucleic acid chain; and, in step (3), at or downstream of the single-strand break, the nucleotide labeled with the first labeling molecule is introduced, and optionally, the nucleotide labeled with the second labeling molecule is introduced, thereby resulting in a labeled product comprising the first labeling molecule and optionally a second labeling molecule. Subsequently, step (4) to step (5) can be carried out as described above, so as to determine the editing site, editing efficiency or off-target effect of the adenine base editor editing the target nucleic acid.

In some preferred embodiments, in step (2), endonuclease V is used to generate a single-strand break downstream of the edited base in the first nucleic acid strand; or, endonuclease VIII is used to generate a single-strand break at the position of the edited base in the first nucleic acid strand.

In such embodiments, the inosine in the labeled product will be read as guanine (G) during the sequencing process, thus, the A-to-G base mutation signal will be generated in the sequencing results of the labeled product. By detecting the base mutation signal, the edited base can be precisely positioned. Thus, in such embodiments, the use of the nucleotide labeled with the second labeling molecule is not necessary. Therefore, in certain exemplary embodiments, in step (3), the nucleotide labeled with the second labeling molecule is not introduced at or downstream of the single-strand break.

However, it is easy to understand that the nucleotide labeled with the second labeling molecule can be used to further amplify the base mutation signal and improve the detection sensitivity. Therefore, in certain exemplary embodiments, in step (3), the nucleotide labeled with the second labeling molecule is introduced at or downstream of the single-strand break.

It is also easy to understand that the above detailed description of the nucleotide labeled with the second labeling molecule is also applicable here. For example, in certain preferred embodiments, the nucleotide labeled with the second labeling molecule is selected from the group consisting of d5fC (5-formylcytosine deoxyribonucleotide), d5caC (5-carboxycytosine deoxyribonucleotide), d5hmC (5-hydroxymethylcytosine deoxyribonucleotide), and dac⁴C (N4-acetylcytosine deoxyribonucleotide).

Furthermore, as described above, in embodiments using the nucleotide labeled with the second labeling molecule, preferably after step (3), the labeled product is treated to alter the complementary base pairing capability of the nucleotide labeled with the second labeling molecule comprised therein; and/or, before step (3) (e.g., before step (2)), the nucleotide labeled with the second labeling molecule possibly existing in the edit product is protected. Regarding the treatment and protection of the nucleotide labeled with the second labeling molecule, the detailed description above is referred.

### Dual Base Editor and Evaluation Thereof

In a preferred embodiment, the base editor is a dual base editor.

In certain preferred embodiments, the base editor is a base editor capable of editing cytosine to uracil and adenine to inosine.

In some preferred embodiments, the edited base is inosine and/or uracil.

In some preferred embodiments, the base editing intermediate is a nucleic acid molecule (e.g., a DNA molecule) comprising inosine and/or uracil.

It is easy to understand that the edit product of editing the target nucleic acid by the dual base editor (e.g., adenine and cytosine dual base editor) also comprises an edited base identical to the edited base generated by editing the target nucleic acid with single base editor (e.g., cytosine base editor and adenine base editor). Therefore, what has been described above for cytosine base editor and adenine base editor and evaluation thereof is also applicable to the adenine and cytosine dual base editor.

In certain preferred embodiments, the protocol described above for cytosine base editor is used to detect the editing site, editing efficiency or off-target effect of the dual base editor (e.g., adenine and cytosine dual base editor) editing the target nucleic acid. For example, the protocol can be used to detect the editing site, editing efficiency or off-target effect of the dual base editor (e.g., adenine and cytosine dual base editor) editing cytosine in the target nucleic acid.

In certain preferred embodiments, the protocol described above for adenine base editor is used to detect the editing site, editing efficiency or off-target effect of the dual base editor (e.g., adenine and cytosine dual base editor) editing the target nucleic acid. For example, the described protocol can be used to detect the editing site, editing efficiency or off-target effect of the dual base editor (e.g., adenine and cytosine dual base editor) editing adenine in the target nucleic acid.

In one aspect, the present application also provides a kit, which comprises an enzyme or a combination of enzymes capable of generating a single-strand break in a segment comprising an edited base, a nucleotide labeled with a first labeling molecule and a first binding molecule capable of specifically recognizing and binding to the first labeling molecule; wherein, the endonuclease or combination of enzymes is capable of specifically recognizing a base editing intermediate comprising the edited base, and capable of generating a phosphodiester bond break in a segment at or upstream 10 nt (e.g., 10 nt, 9 nt, 8 nt, 7 nt, 6 nt, 5 nt, 4 nt, 3 nt, 2 nt, 1 nt) to downstream 10 nt (e.g., 10 nt, 9 nt, 8 nt, 7 nt, 6 nt, 5 nt, 4 nt, 3 nt, 2 nt, 1 nt) of the edited base.

In some preferred embodiments, the enzyme or combination of enzymes capable of generating a single-strand break in the segment comprising the edited base is endonuclease V, or endonuclease VIII.

In certain preferred embodiments, the enzyme or combination of enzymes capable of generating single-strand break in the segment comprising the edited base is a combination of UDG enzyme and AP endonuclease.

In certain preferred embodiments, the kit further comprises a nucleotide labeled with a second labeling molecule, the nucleotide labeled with the second labeling molecule is a nucleotide molecule that is capable of complementary base pairing with different nucleotides under different conditions (e.g., before and after a treatment). In certain preferred embodiments, the nucleotide labeled with the second labeling molecule is selected from the group consisting of d5fC (5-formylcytosine deoxyribonucleotide), d5caC (5-carboxycytosine deoxyribonucleotide), d5hmC (5-hydroxymethylcytosine deoxyribonucleotide), and dac⁴C (N4-acetylcytosine deoxyribonucleotide).

In certain preferred embodiments, the nucleotide labeled with the second labeling molecule is a modified cytosine deoxyribonucleotide, which is capable of undergoing complementary base pairing with a first nucleotide (e.g., guanine deoxyribonucleotide) before undergoing a treatment, and capable of complementary base pairing with a second nucleotide (e.g., adenine deoxyribonucleotide) after undergoing a treatment. In some preferred embodiments, the nucleotide labeled with the second labeling molecule is selected from the group consisting of d5fC (5-formylcytosine deoxyribonucleotide), d5caC (5-carboxycytosine deoxyribonucleotide), d5hmC (5-hydroxymethylcytosine deoxyribonucleotide) and dac⁴C (N4-acetylcytosine deoxyribonucleotide).

In certain preferred embodiments, the kit further comprises a reagent (e.g., ethylhydroxylamine, reagents (e.g., β-glucosyltransferase, glucosyl compound) required for glycosylation reaction catalyzed by βGT, or any combination thereof) for protecting the nucleotide labeled with the second labeling molecule, and/or, a reagent (e.g., malononitrile, azido-indandione, borane compound (e.g., pyridine borane compound, such as pyridine borane or 2-picoline borane), potassium ruthenate. TET protein, sodium cyanoborohydride, or any combination thereof) for treating the nucleotide labeled with the second labeling molecule to alter complementary base-pairing capability thereof.

In certain preferred embodiments, the nucleotide labeled with the second labeling molecule is 5-formylcytosine deoxyribonucleotide. In such embodiments, the kit may further comprise a reagent (e.g., ethylhydroxylamine) for protecting the nucleotide labeled with the second labeling molecule, and/or, a reagent (e.g., malononitrile, borane compound (e.g., pyridine borane compound, such as pyridine borane or 2-picoline borane), or azido-indandione) for treating the nucleotide labeled with the second labeling molecule to alter complementary base pairing capability thereof.

In certain preferred embodiments, the nucleotide labeled with the second labeling molecule is 5-hydroxymethylcytosine deoxyribonucleotide. In such embodiments, the kit may further comprise a reagent (e.g., reagents (e.g., β-glucosyltransferase, glucosyl compound) required for glycosylation reaction catalyzed by βGT) for protecting the nucleotide labeled with the second labeling molecule, and/or, a reagent (e.g., potassium ruthenate or TET protein, and malononitrile or borane compound (e.g., pyridine borane compound such as pyridine borane or 2-picoline borane) or azido-indandione) for treating the nucleotide labeled with the second labeling molecule to alter complementary base pairing capability thereof.

In certain preferred embodiments, the nucleotide labeled with the second labeling molecule is 5-carboxycytosine deoxyribonucleotide. In such embodiments, the kit may further comprise a reagent (e.g., borane compound (e.g., pyridine borane compound, such as pyridine borane or 2-picoline borane)) for treating the nucleotide labeled with the second labeling molecule to alter complementary base pairing capability thereof.

In certain preferred embodiments, the nucleotide labeled with the second labeling molecule is N4-acetylcytosine deoxyribonucleotide. In such embodiments, the kit may further comprise a reagent (e.g., sodium cyanoborohydride) for treating the nucleotide labeled with the second labeling molecule to alter complementary base pairing capability thereof.

In certain preferred embodiments, the kit further comprises a nucleic acid polymerase (e.g., a nucleic acid polymerase with strand displacement activity), a nucleic acid ligase (e.g., a DNA ligase), an unlabeled nucleotide molecule, a reagent (e.g., ethylhydroxylamine, reagents (e.g., β-glucosyltransferase, glucosyl compound) required for βGT-catalyzed glycosylation reaction, or any combination thereof) for protecting the nucleotide labeled with the second labeling molecule, a reagent (e.g., malononitrile, azido-indandione, borane compound (e.g., pyridine borane compound, such as pyridine borane, or 2-picoline borane), potassium ruthenate. TET protein, sodium cyanoborohydride, or any combination thereof) for treating the nucleotide labeled with the second labeling molecule to alter complementary base pairing capability thereof, or any combination thereof.

It is readily understood that the kit is used to carry out the method of the present application. Therefore, the detailed descriptions above for the base editor (e.g., single base editor and dual base editor), the first labeling molecule, the first binding molecule, the nucleotide labeled with the first labeling molecule, the second labeling molecule, the nucleotidelabeled with the second labeling molecule, the nucleic acid polymerase, the nucleic acid ligase, the UDG enzyme, the AP endonuclease, the endonuclease V or VIII, and the like are also applicable here.

In some preferred embodiments, the kit is used to detect the editing site, editing efficiency or off-target effect of a base editor (e.g., a single base editor or a dual base editor) editing a target nucleic acid.

In some preferred embodiments, the kit is used to detect the editing site, editing efficiency or off-target effect of a cytosine base editor editing a target nucleic acid. In certain preferred embodiments, the kit comprises, a UDG enzyme, an AP endonuclease, a nucleotide labeled with a first labeling molecule, a first binding molecule and a nucleotide labeled with a second labeling molecule (e.g. d5fC, d5caC, d5hmC or dac⁴C); optionally further comprises, a nucleic acid polymerase, a nucleic acid ligase, an unlabeled nucleotide molecule, a reagent (e.g., ethylhydroxylamine, reagents (e.g., β-glucosyltransferase, glucosyl compound) required for βGT-catalyzed glycosylation reaction, or any combination thereof) for protecting the nucleotide labeled with the second labeling molecule, a reagent (e.g., malononitrile, azido-indandione, borane compound (e.g., pyridine borane compound, such as pyridine borane or 2-picoline borane), potassium ruthenate, TET protein, sodium cyanoborohydride, or any combination thereof) for treating the nucleotide labeled with the second labeling molecule to alter complementary base pairing capability thereof, or any combination thereof.

In some preferred embodiments, the kit is used to detect the editing site, editing efficiency or off-target effect of an adenine base editor editing a target nucleic acid. In certain preferred embodiments, the kit comprises, an endonuclease V or VIII, a nucleotide labeled with a first labeling molecule, and a first binding molecule; optionally comprises, a nucleic acid polymerase, a nucleic acid ligase, a nucleotide labeled with a second labeling molecule (e.g., d5fC, d5caC, d5hmC or dac⁴C), an unlabeled nucleotide molecule, a reagent (e.g., ethylhydroxylamine, reagents (e.g., β-glucosyltransferase, glucosyl compound) required for βGT-catalyzed glycosylation reaction, or any combination thereof) for protecting the nucleotide labeled with the second labeling molecule, a reagent (e.g., malononitrile, azido-indandione, borane compound (e.g., pyridine borane compound, such as pyridine borane or 2-picoline borane), potassium ruthenate, TET protein, sodium cyanoborohydride, or any combination thereof) for treating the nucleotide labeled with the second labeling molecule to alter complementary base pairing capability thereof, or any combination thereof.

In some preferred embodiments, the kit is used to detect the editing site, editing efficiency or off-target effect of a dual base editor (e.g., an adenine and cytosine dual base editor) editing a target nucleic acid. In certain preferred embodiments, the kit comprises, a UDG enzyme, an AP endonuclease, an endonuclease V or VIII, a nucleotide labeled with a first labeling molecule, a first binding molecule and a nucleotide labeled with a second labeling molecule (e.g., d5fC, d5caC, d5hmC, or dac⁴C); optionally further comprises, a nucleic acid polymerase, a nucleic acid ligase, an unlabeled nucleotide molecule, a reagent (e.g., ethylhydroxylamine, reagents (e.g., β-glucosyltransferase, glucosyl compound) required for βGT-catalyzed glycosylation reaction, or any combination thereof) for protecting the nucleotide labeled with the second labeling molecule, a reagent (e.g., malononitrile, azido-indandione, borane compound (e.g., pyridine borane compound, such as pyridine borane or 2-picoline borane), potassium ruthenate. TET protein, sodium cyanoborohydride, or any combination thereof) for treating the nucleotide labeled with the second labeling molecule to alter complementary base pairing capability thereof, or any combination thereof.

### Definition of Terms

In the present application, unless otherwise stated, scientific and technical terms used herein have the meanings commonly understood by those skilled in the art. Moreover, the nucleic acid chemistry laboratory operation steps used herein are all routine steps widely used in the corresponding field. Meanwhile, in order to better understand the present invention, definitions and explanations of relevant terms are provided below. Unless specifically defined or described differently elsewhere herein, the following terms and descriptions related to the present invention are to be read in accordance with the definitions given below.

When the terms “for example”, “such as”, “e.g.,”, “including”, “comprising” or variations thereof are used herein, these terms will not be considered as terms of limitation, but will be construed to mean “but not limited to” or “not limited to”.

Unless otherwise indicated herein or clearly contradicted by context, the terms “a” and “an” as well as “the” and similar designations in the context of describing the present invention (especially in the context of the following claims) are to be construed to cover singular and plural.

As used herein, the term “base editor” refers to a reagent comprising a polypeptide capable of editing or modifying a base (e.g., A, T, C, G or U) in a nucleic acid molecule (e.g., DNA or RNA). In some embodiments, the base editor is a single base editor or a dual base editor.

In some embodiments, the base editor is a single base editor, which is capable of editing one kind of base within a nucleic acid molecule (e.g., a DNA molecule); for example, which is capable of deaminating one kind of base within a nucleic acid molecule (e.g., a DNA molecule). In some embodiments, the single base editor is capable of deaminating adenine (A) in DNA. In some embodiments, the single base editor is capable of deaminating cytosine (C) in DNA. In some embodiments, the single base editor comprises an adenosine deaminase and a nucleic acid-programmable DNA-binding protein (napDNAbp), for example, is a fusion protein comprising a nucleic acid-programmable DNA-binding protein (napDNAbp) fused to adenosine deaminase. In some embodiments, the single base editor comprises a cytidine deaminase and a nucleic acid-programmable DNA-binding protein (napDNAbp), for example, is a fusion protein comprising napDNAbp fused to cytidine deaminase. In some embodiments, the nucleic acid-programmable DNA-binding protein (napDNAbp) is a Cas9 protein, such as Cas9 Nickase (nCaS9) that can only cut one strand of a nucleic acid duplex or Cas9 (dCaS9) without nuclease activity.

In some embodiments, the single base editor comprises an adenosine deaminase and a Cas9) protein, for example, a Cas9 protein fused to the adenosine deaminase. In some embodiments, the single base editor comprises a cytidine deaminase and a Cas9 protein, for example, a Cas9 protein fused to the cytidine deaminase. In some embodiments, the single base editor comprises an adenosine deaminase and a nCaS9, for example, a nCaS9 fused to the adenosine deaminase. In some embodiments, the single base editor comprises a cytidine deaminase and a nCaS9, for example, a nCaS9 fused to the cytidine deaminase. In some embodiments, the single base editor comprises an adenosine deaminase and a dCaS9, for example, a dCaS9 fused to the adenosine deaminase. In some embodiments, the single base editor comprises a cytidine deaminase and a dCaS9, for example, a dCaS9 fused to the cytidine deaminase.

In some embodiments, the base editor is a dual base editor, which is capable of editing two kinds of bases within a nucleic acid molecule (e.g., a DNA molecule); for example, which is capable of deaminating two kinds of bases within a nucleic acid molecule (e.g., a DNA molecule). In some embodiments, the dual base editor is capable of deaminating adenine (A) and cytosine (C) in DNA. In some preferred embodiments, the dual base editor is capable of deaminating adenine (A) and cytosine (C) in DNA located within the same editing window. In some embodiments, the dual base editor comprises an adenosine deaminase, a cytidine deaminase, and a nucleic acid-programmable DNA-binding protein (napDNAbp). In some embodiments, the nucleic acid-programmable DNA-binding protein (napDNAbp) is a Cas9 protein, such as a Cas9 Nickase (nCaS9) that can only cut one strand of a nucleic acid duplex or a Cas9 (dCaS9) without nuclease activity. In some embodiments, the dual base editor comprises an adenosine deaminase, a cytidine deaminase, and a Cas9 protein. In some embodiments, the dual base editor comprises an adenosine deaminase, a cytidine deaminase, and a Cas9 Nickase (nCaS9). In some embodiments, the dual base editor comprises adenosine deaminase, cytidine deaminase, and a Cas9 without nuclease activity (dCaS9). In some embodiments, the dual base editor is a complex or fusion protein comprising an adenosine deaminase, a cytidine deaminase and a napDNAbp.

It is easy to understand that the dual base editor may comprise one or more (e.g., one or two) nucleic acid-programmable DNA-binding proteins (napDNAbps). In some embodiments, the dual base editor comprises two napDNAbps which are independently fused to adenosine deaminase and cytidine deaminase, respectively. In some embodiments, the dual base editor comprises one napDNAbp which is fused to both adenosine deaminase and cytidine deaminase. In some embodiments, the dual base editor is a combination of two single base editors.

In some embodiments, the base editor is fused to a base excision repair inhibitor (e.g., a UGI domain or a DISN domain). In some embodiments, the fusion protein comprises a nCas9 and a base excision repair inhibitor, such as UGI or DISN domain, fused to a deaminase. In some embodiments, the base excision repair inhibitor, such as UGI domain or DISN domain, is provided in the system, but not fused to a Cas9 protein (or dCas9, nCas9). It should be emphasized that the term “fused with” or “fused to” here comprises fusion or ligation between proteins (or functional domains thereof) with or without linkers. In certain embodiments, the “linker” is a peptide linker. In certain embodiments, the “linker” is a non-peptide linker.

In some embodiments, the deaminase and the nucleic acid-programmable DNA-binding protein comprised in the base editor are structurally independent between each other, that is, the deaminase and the nucleic acid-programmable DNA-binding protein comprised in the base editor are not fused or ligated by a linker. In certain embodiments, the deaminase and the nucleic acid-programmable DNA-binding protein comprised in the base editor are non-covalently linked or bound.

It is easy to understand that the deaminase may be a specific deaminase directed to a glycoside formed by any base or a combination thereof (e.g., adenosine deaminase, cytidine deaminase).

In some embodiments, the nucleic acid-programmable DNA-binding protein can be selected from the group consisting of TALES, ZFs, Casx, Casy, Cpf1, C2c1, C2c2, C2c3, Argonaute protein, or derivative thereof. In certain embodiments, the programmable DNA-binding protein does not have nuclease activity. In certain embodiments, the programmable DNA-binding protein can cut only one strand of a nucleic acid duplex. In certain embodiments, the programmable DNA-binding protein does not have the activity of forming a nucleic acid double-strand break.

In certain embodiments, the base editor is a cytosine base editor, such as cytosine base editor BE3, cytosine base editor upgraded version BE4max, mitochondrial cytosine base editor DdCBE, and various CBE editing systems. For a description of various cytosine base editors, see, for example, Andrew V. Anzalone, et al. Nature biotechnology 38(7), 824-844, doi: 10.1038/s41587-020-0561-9 (2020), which is hereby incorporated by reference in its entirety.

In some embodiments, the base editor is an adenine base editor, such as adenine base editor ABE7.10, adenine base editor ABEmax and adenine base editor ABE8e, and various ABE editing systems. For a detailed description of various adenine base editors, see, for example, Andrew V. Anzalone, et al. Nature biotechnology 38(7), 824-844, doi: 10.1038/s41587-020-0561-9 (2020), which is incorporated herein by reference in its entirety.

In certain embodiments, the base editor is a base editor capable of editing adenine and cytosine, such as ACBE.

As used herein, the term “base editing intermediate” refers to a product of a base editor (e.g., a single base editor or a dual base editor) editing a target nucleic acid, which comprises an edited base generated by the base editor editing the target nucleic acid. The target nucleic acid can be derived from any organism (e.g., eukaryotic cells, prokaryotic cells, viruses and viroids) or non-biological organism (e.g., libraries of nucleic acid molecules). In certain embodiments, the base editing intermediate is a direct product of a base editor editing a target nucleic acid. In some embodiments, the base editing intermediate is a product obtained by enrichment and/or nucleic acid fragmentation of a direct product of a base editor editing a target nucleic acid. In some embodiments, the edited base (e.g., uracil, inosine) is a base modified by the corresponding active element (e.g., cytidine deaminase, adenosine deaminase) in the base editor. Generally speaking, bases before and after modification/editing have different complementary base pairing capabilities (i.e., capabilities of complementary pairing with different bases). For example, cytosine in a nucleic acid is edited by cytidine deaminase in a base editor and converted into uracil, and uracil is complementary to adenine instead of guanine. For example, adenine in a nucleic acid is edited by adenosine deaminase in a base editor and converted into inosine, and inosine is complementary to cytosine instead of thymine.

As used herein, the term “borane compound” refers to a borane compound that can be used to treat the nucleotide labeled with the second labeling molecule of the present application to change complementary base pairing capability thereof. In particular, it can be a pyridine borane compound, which comprises pyridine borane and derivatives thereof. Non-limiting examples of such pyridine borane are pyridine borane, 2-picoline borane (see, for example, Liu, Y. et al. Bisulfite-free direct detection of 5-methylcytosine and 5-hydroxymethylcytosine at base resolution. Nature biotechnology 37, 424-429, doi:10.1038/s41587-019-0041-2 (2019)., which is incorporated herein by reference in its entirety).

As used herein, the term “upstream” is used to describe the relative positional relationship of two nucleic acid sequences (or two nucleic acid molecules), and has the meaning generally understood by those skilled in the art. For example, the expression “one nucleic acid sequence is located upstream of another nucleic acid sequence ” means that, when arranged in the 5′ to 3′ direction. the former is located at a more front position than the latter (i.e., a position closer to the 5′ end). As used herein, the term “downstream” has the opposite meaning of “upstream”.

As used herein, the term “first labeling molecule” refers to a molecule capable of specifically forming an interacting molecular pair with a first binding molecule. According to the method of the present application, the specific binding of the first binding molecule to the first labeling molecule can be used to enrich the labeled product comprising the first labeling molecule. In certain embodiments, the first labeling molecule binds reversibly or irreversibly to the first binding molecule. In certain preferred embodiments, the first labeling molecule binds reversibly to the first binding molecule.

As used herein, the term “nucleotide labeled with first labeling molecule” refers to a nucleotide molecule comprising a group in the first labeling molecule capable of specifically forming an interaction molecular pair with a first binding molecule. In some preferred embodiments, the nucleotide labeled with the first labeling molecule refers to a single nucleotide molecule, such as dUTP, dATP, dTTP, dCTP or dGTP labeled with the first labeling molecule, or any combination thereof.

In some embodiments, the labeled nucleotide molecule is reversibly or irreversibly linked to the first labeling molecule. In some embodiments, a ribose, base, or phosphate moiety of the labeled nucleotide molecule is reversibly or irreversibly linked to the first labeling molecule. In some preferred embodiments, the labeled nucleotide molecule is reversibly linked to the first labeling molecule. It should be noted that, in some cases, the nucleotide labeled with the first labeling molecule does not comprise the complete structure of the first labeling molecule, but comprises a group in the first labeling molecule capable of specifically forming an interaction molecular pair with a first binding molecule.

As used herein, the term “second labeling molecule” refers to a molecule capable of modifying a base in a nucleotide molecule to produce a modified base, and the modified base is capable of complementary pairing with different bases under different conditions (e.g., before and after undergoing a treatment).

As used herein, the term “nucleotide labeled with second labeling molecule” refers to a nucleotide molecule capable of complementary base pairing with a different nucleotide under different conditions (e.g., before and after undergoing a treatment). In some preferred embodiments, the nucleotide labeled with the second labeling molecule refer to a single nucleotide molecule.

As used herein, a nucleic acid polymerase having “strand displacement activity” refers to a nucleic acid polymerase that, in the process of extending a new nucleic acid strand, when encountering a downstream nucleic acid strand complementary to a template strand, can continue the extension reaction and degrade (rather than strip) the nucleic acid strand complementary to the template strand. In certain preferred embodiments, the nucleic acid polymerase having “strand displacement activity” also has 5′ to 3′ exonuclease activity.

As used herein, “high-fidelity nucleic acid polymerase” refers to a nucleic acid polymerase that, during the process of amplifying nucleic acid, has a probability of introducing erroneous nucleotides (i.e., error rate) lower than that of wild-type Taq enzyme (e.g., Taq enzyme having a sequence set forth in UniProt Accession: P19821.1). For example, Q5® Start High-Fidelity DNA Polymerase.

As used herein, “low-fidelity nucleic acid polymerase” refers to a nucleic acid polymerase that, during the process of amplifying nucleic acid, has a probability of introducing erroneous nucleotides (i.e., error rate) higher than that of wild-type Taq enzyme (e.g., Taq enzyme having a sequence set forth in UniProt Accession: P19821.1). For example, MightyAmp DNA Polymerase.

As used herein, unless the context clearly indicates otherwise, the term “nucleotide” as used herein preferably refers to nucleoside triphosphate, such as deoxyribonucleotide triphosphate.

### Beneficial Effect

The present application provides a new method for detecting the editing site, editing efficiency or off-target effect of a base editor (e.g., cytosine base editor, adenine base editor, adenine and cytosine dual base editor) editing a nucleic acid, which has one or more beneficial technical effects selected from the group consisting of the following:
(1) The method of the present invention can capture a base editing intermediate (e.g., nucleic acid comprising uracil or inosine) produced by a base editing tool in a living cell, so it can obtain the information of a site where a base editing event actually occurs.
(2) The method of the present invention can effectively label and enrich an editing site, so that it can be easily distinguished from genetic background such as SNV and sequencing errors.
(3) In the prior art, when using whole-genome sequencing technology to detect base-edited sites, the coverage of the sequencing reads on the whole genome is very uneven, which requires a huge amount of data to obtain enough information to evaluate editing sites across the genome. The method of the present invention overcomes this defect, and can obtain strong detection signals at the whole genome level with a relatively less amount of data.
(4) The method of the present invention has no preference for various base editing tools (e.g., CBE, ABE). As mentioned earlier, various optimized base editing tools have been developed to meet practical needs. Since the method of the present invention can capture base editing intermediates (e.g., nucleic acids comprising uracil or inosine) produced by various base editing processes, the method of the present invention can be generally applied to various base editing tools to detect the editing sites, and can be used to evaluate the editing efficiency or off-target effect thereof.

The embodiments of the present invention will be described in detail below with reference to the drawings and examples, but those skilled in the art will understand that the following drawings and examples are only for illustrating the present invention, rather than limiting the scope of the present invention. Various objects and advantages of the present invention will become apparent to those skilled in the art from the accompanying drawings and the following detailed description of the preferred embodiment.

### BRIEF DESCRIPTION OF THE DRAWINGS

FIG. 1 shows an exemplary scheme 1 of using the method of the present invention to detect an editing site of a base editor, wherein the base editor is a cytosine base editor.

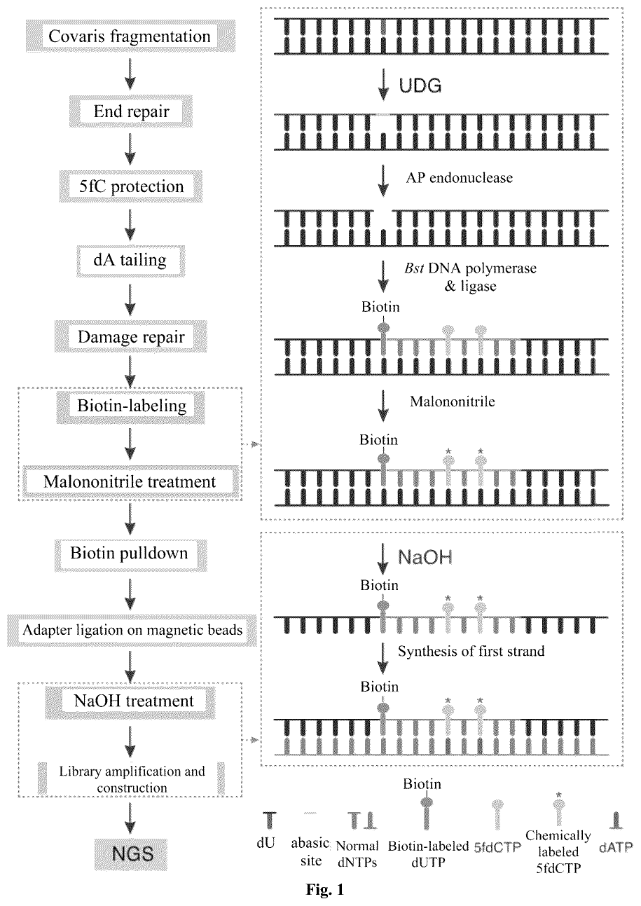

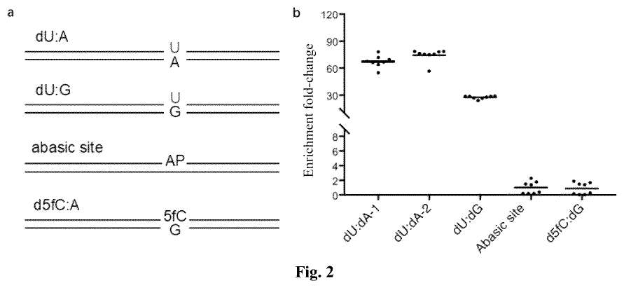

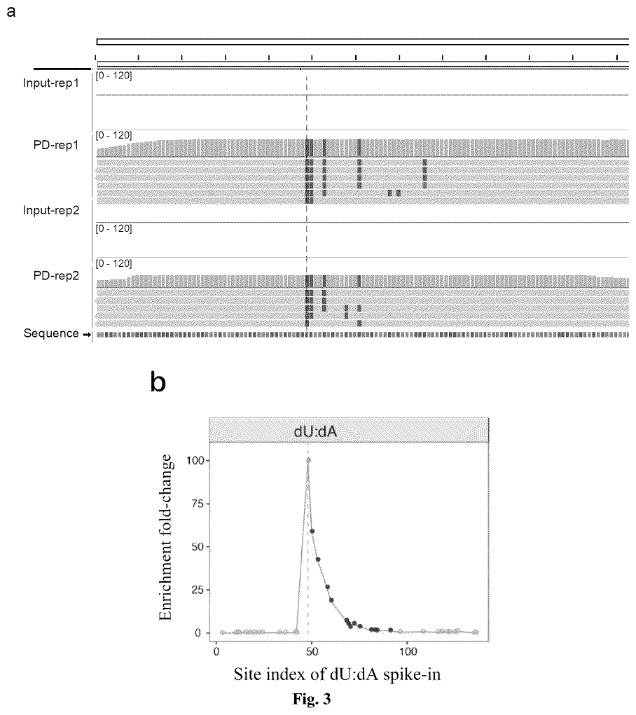

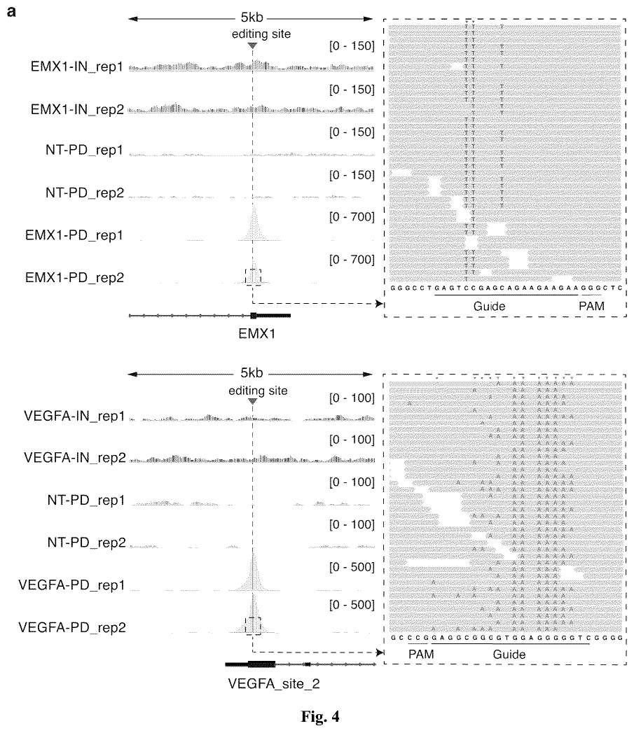

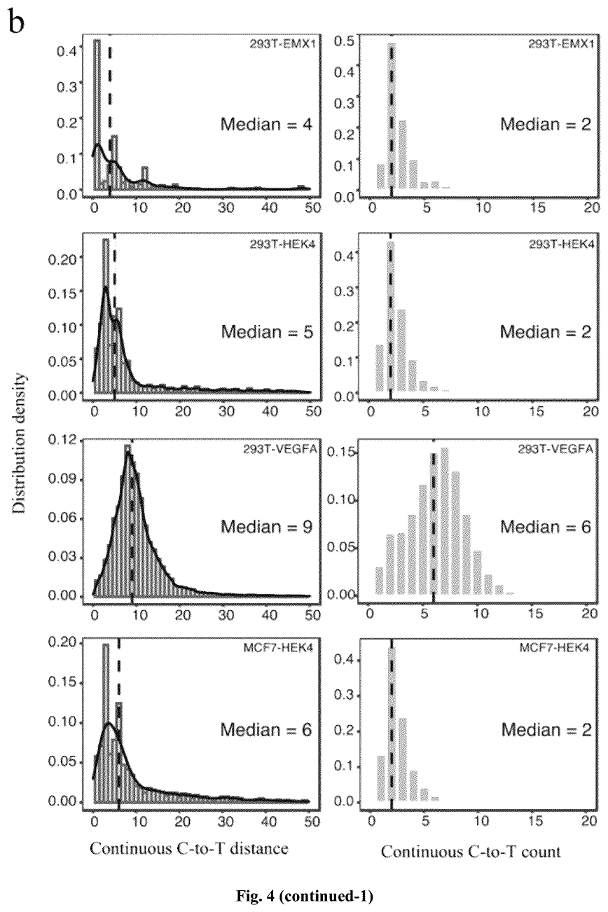

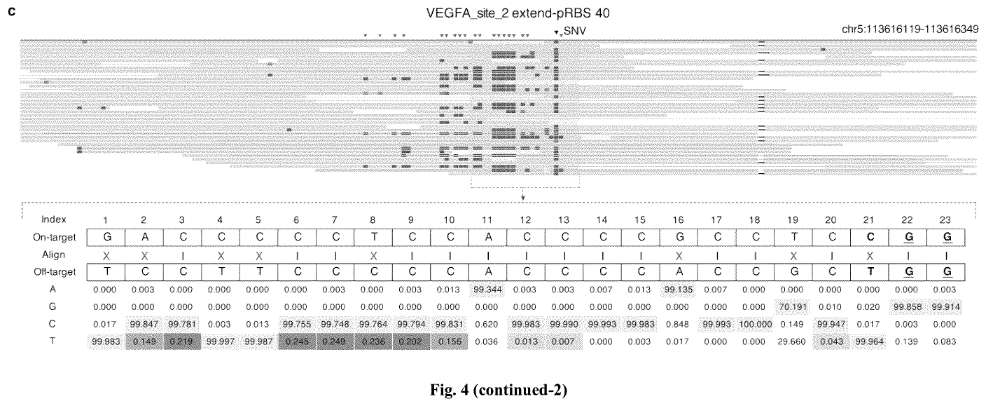

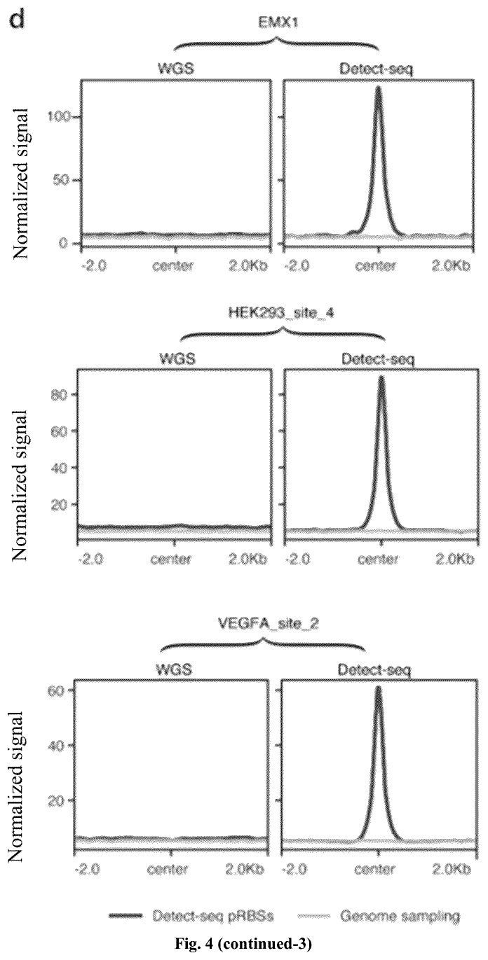

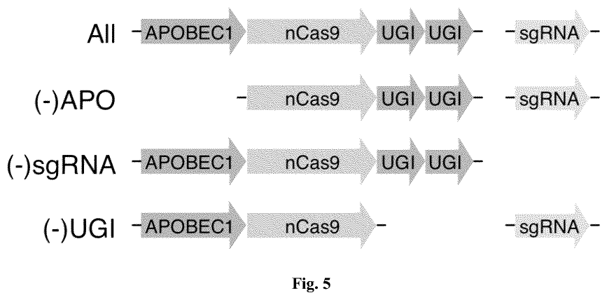

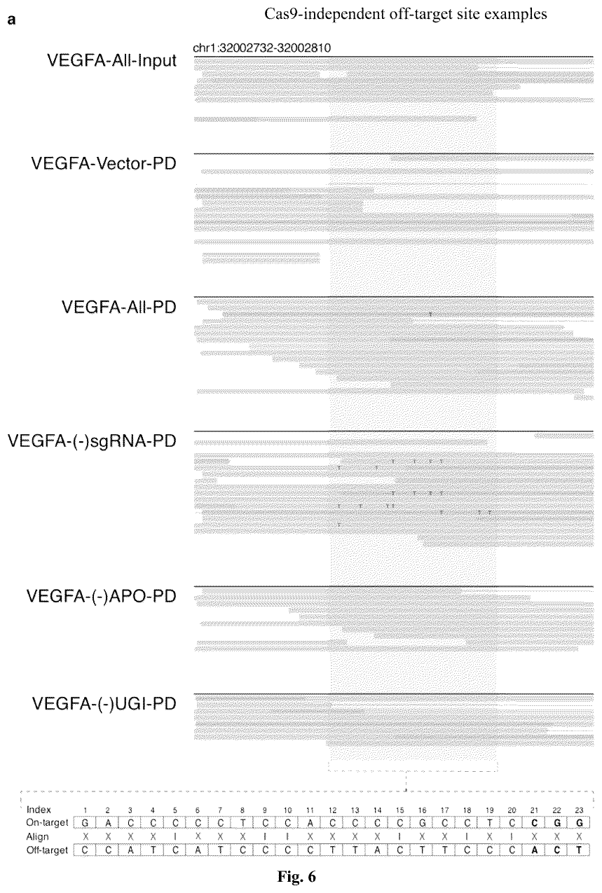

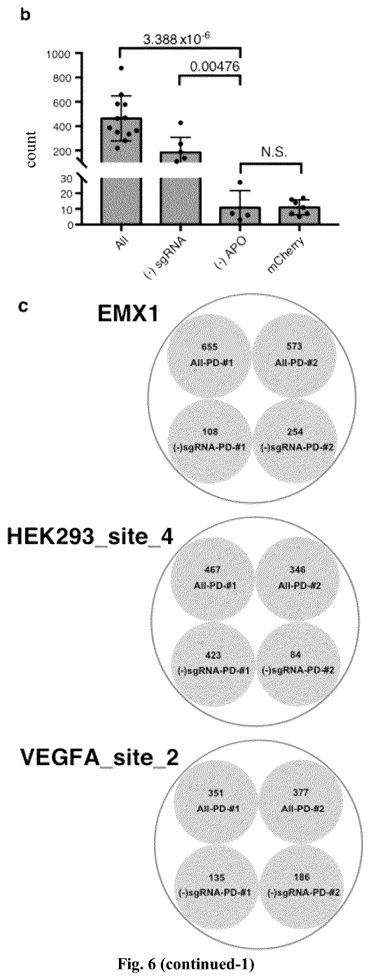

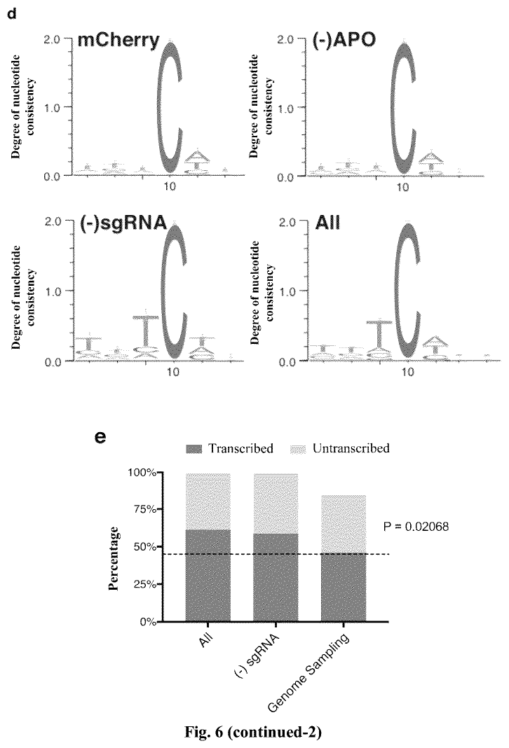

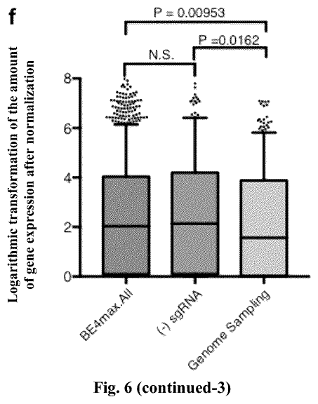

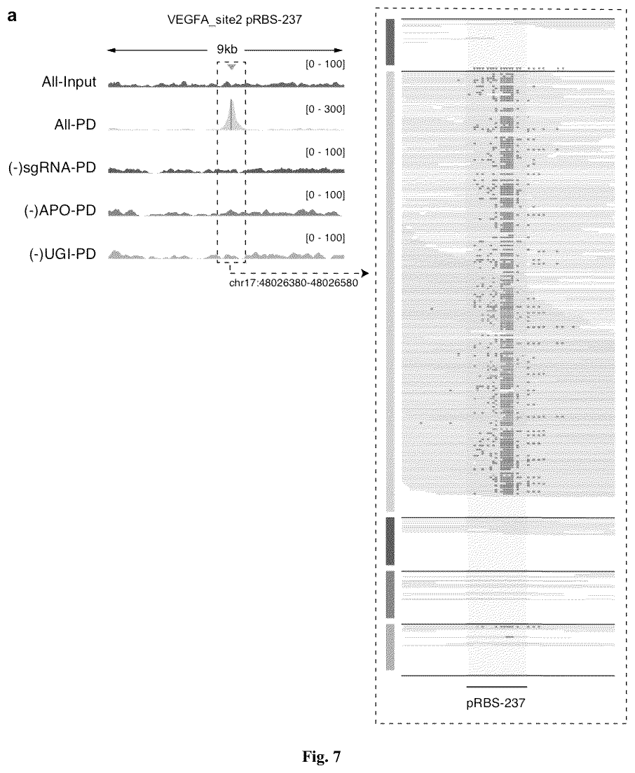

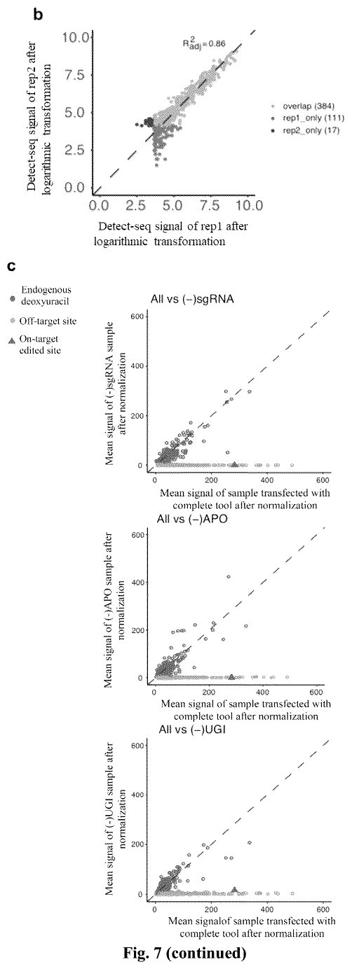

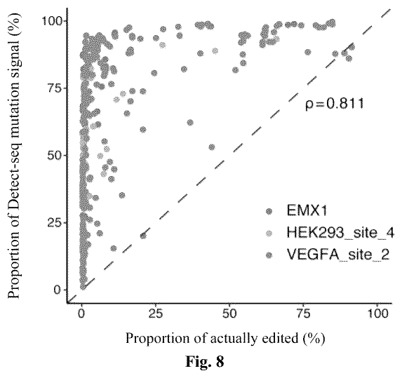

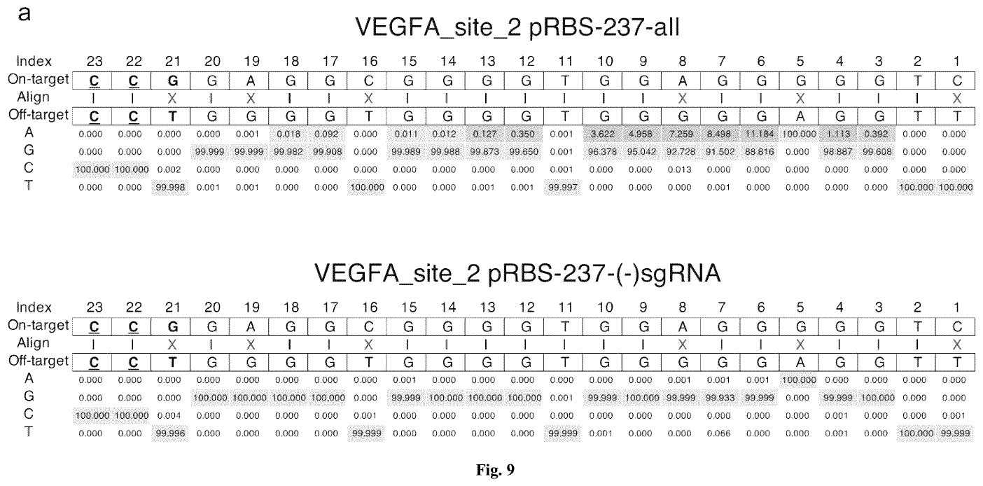

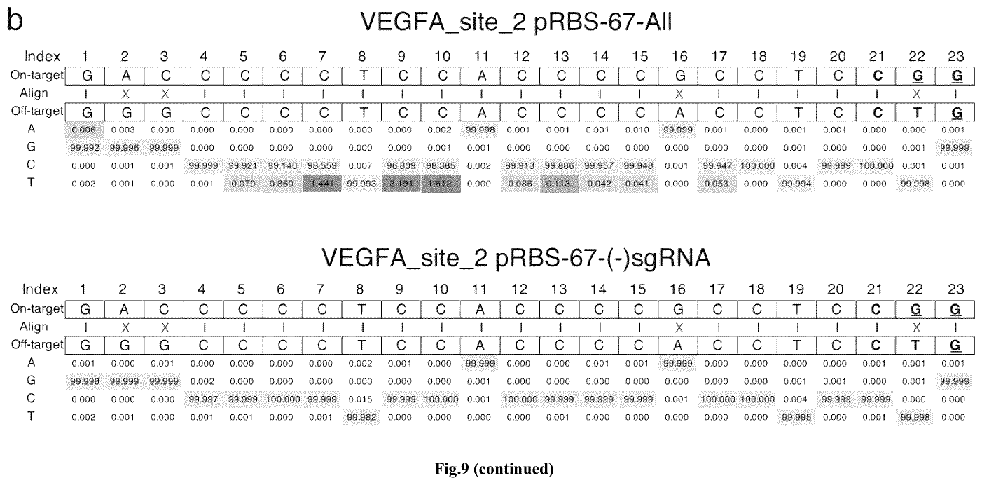

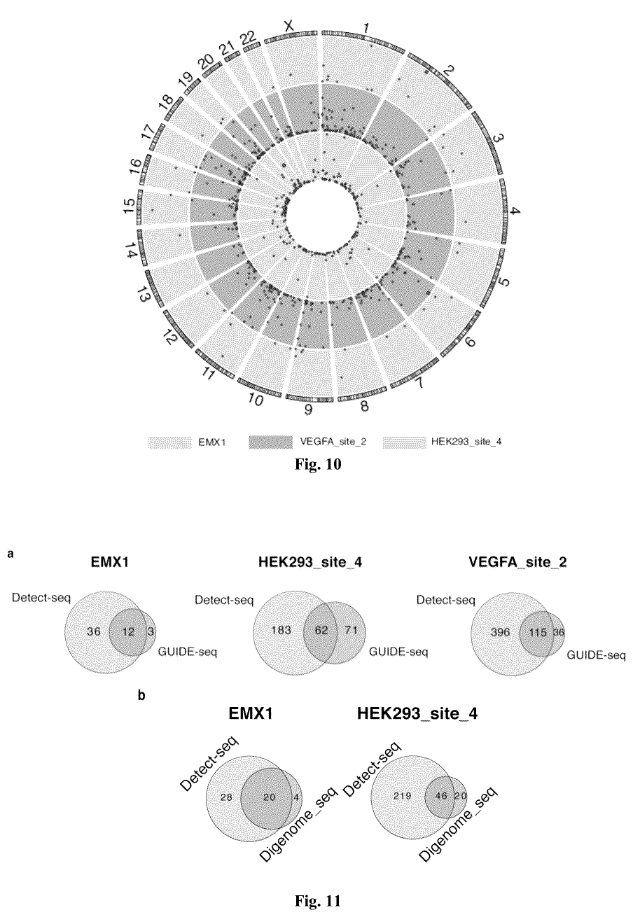

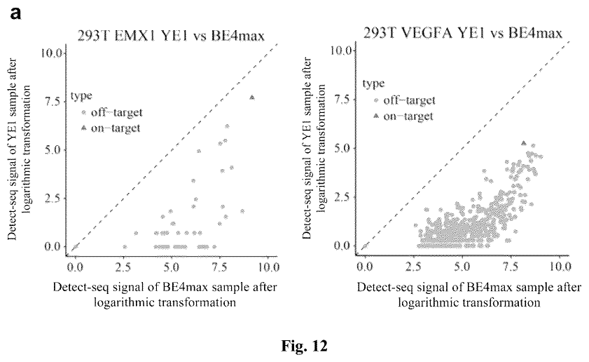

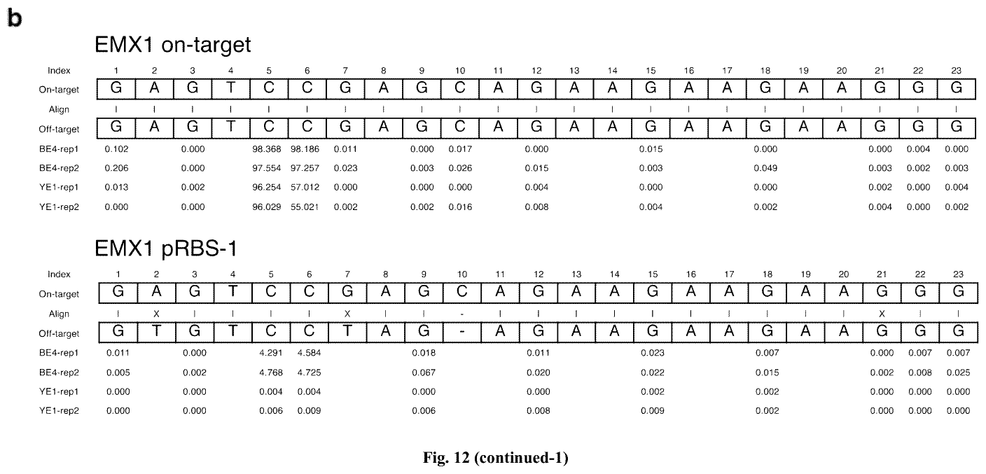

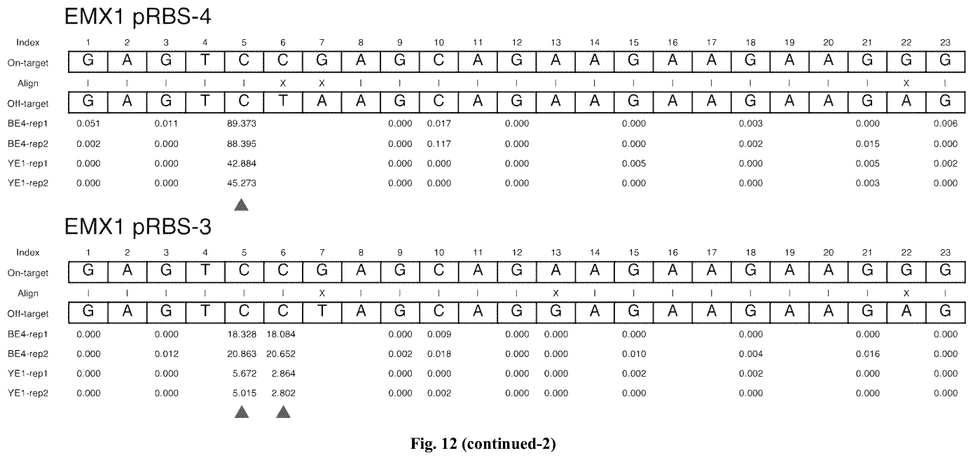

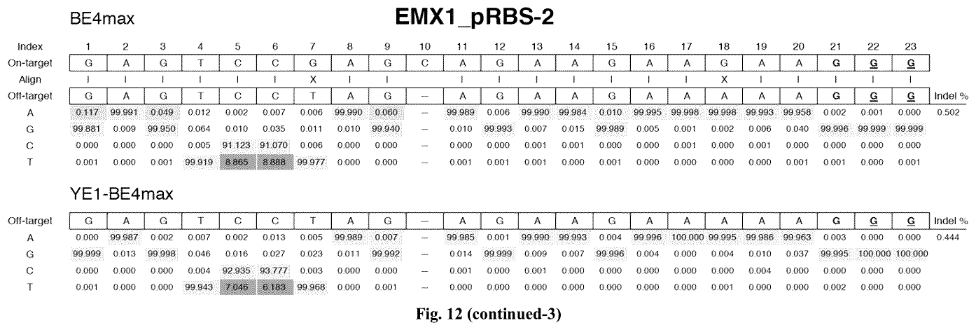

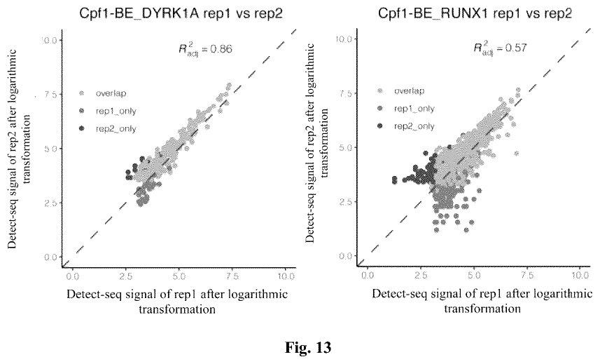

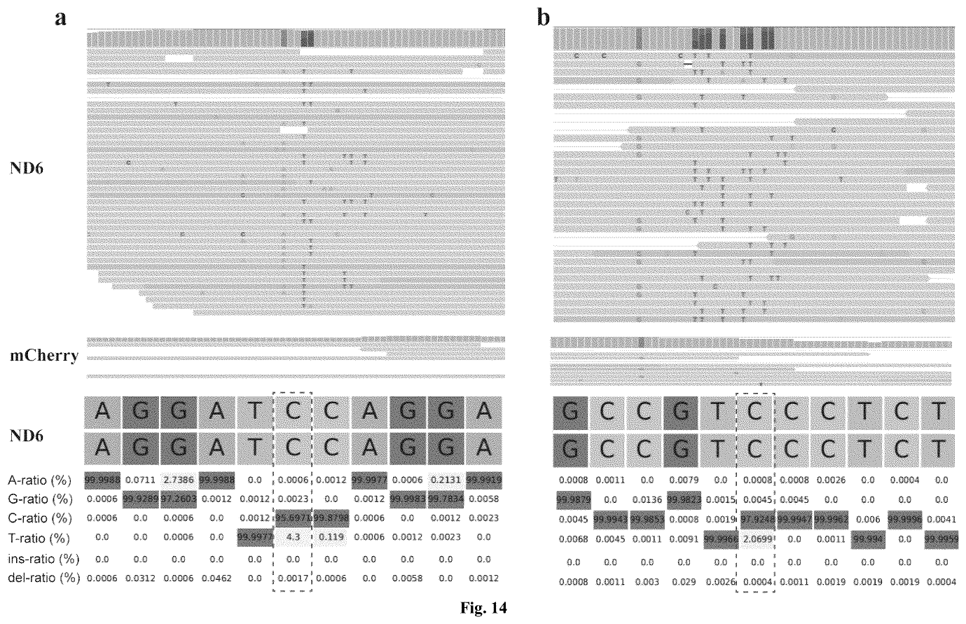

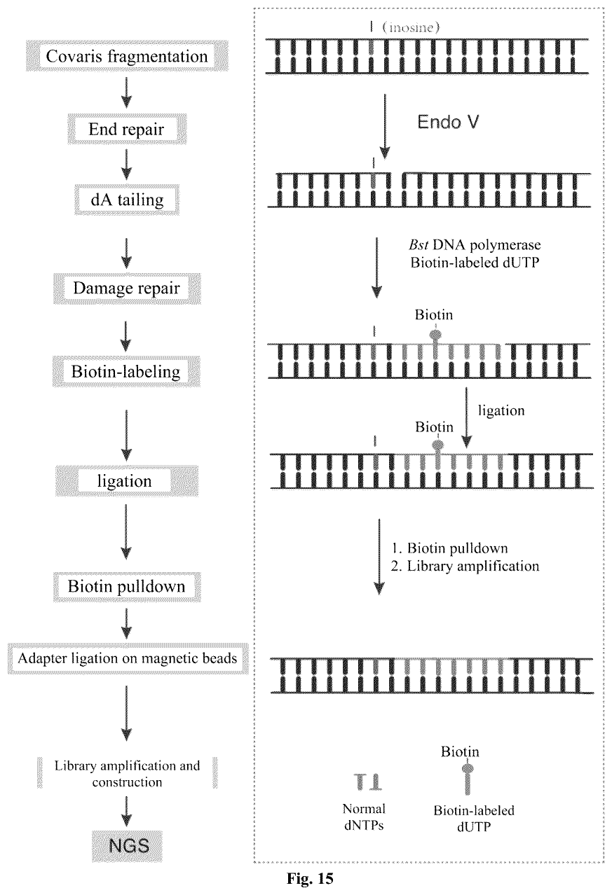

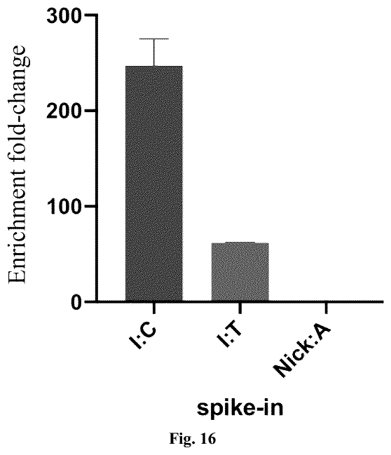

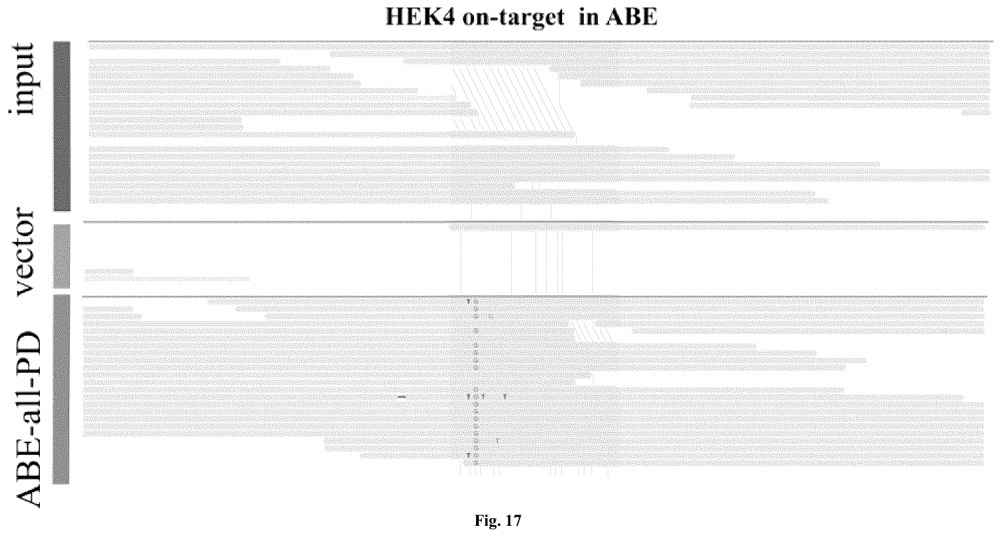

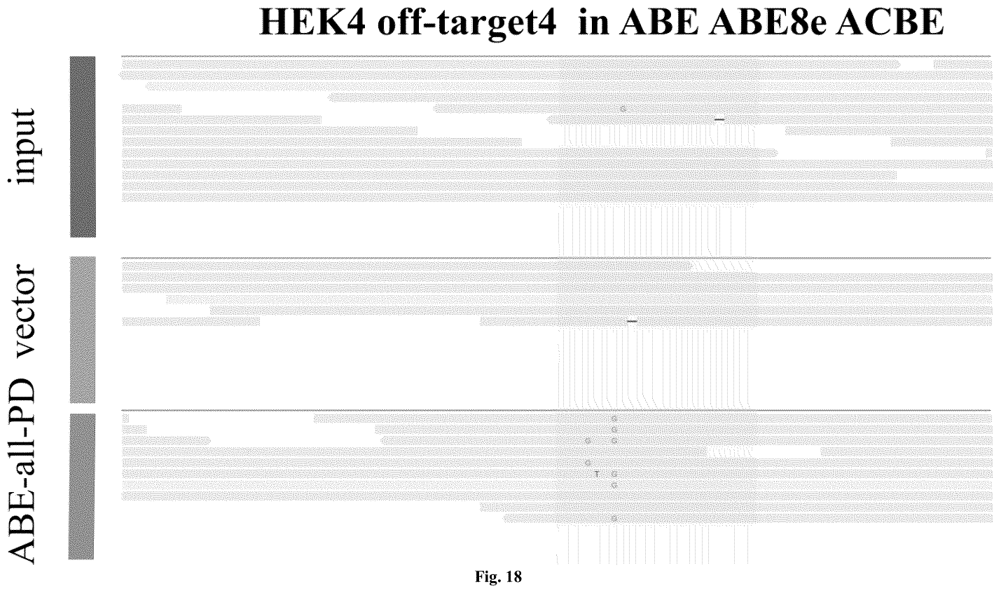

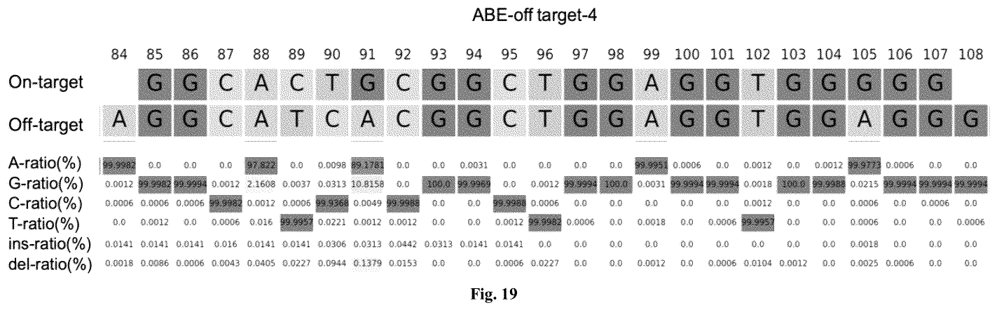

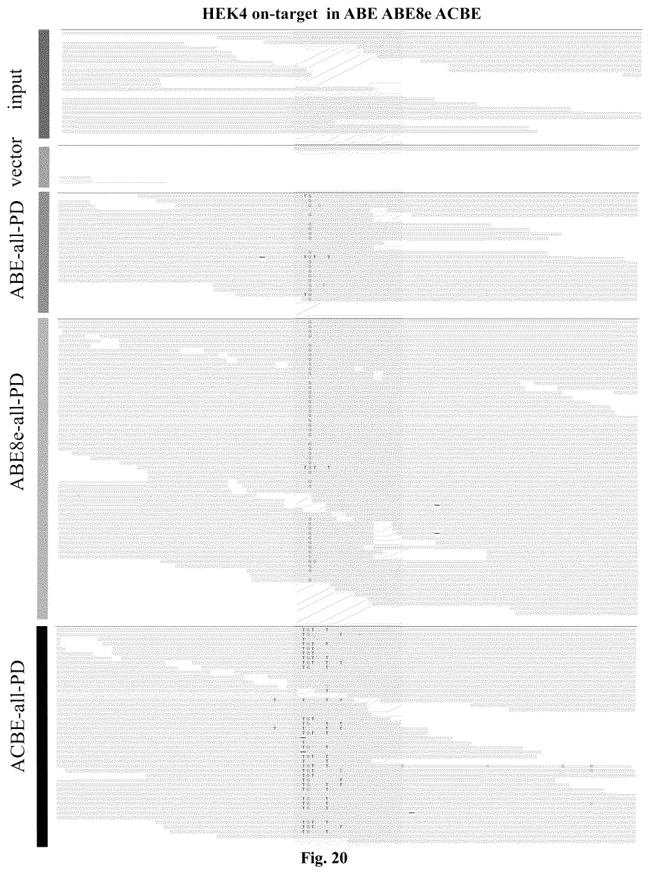

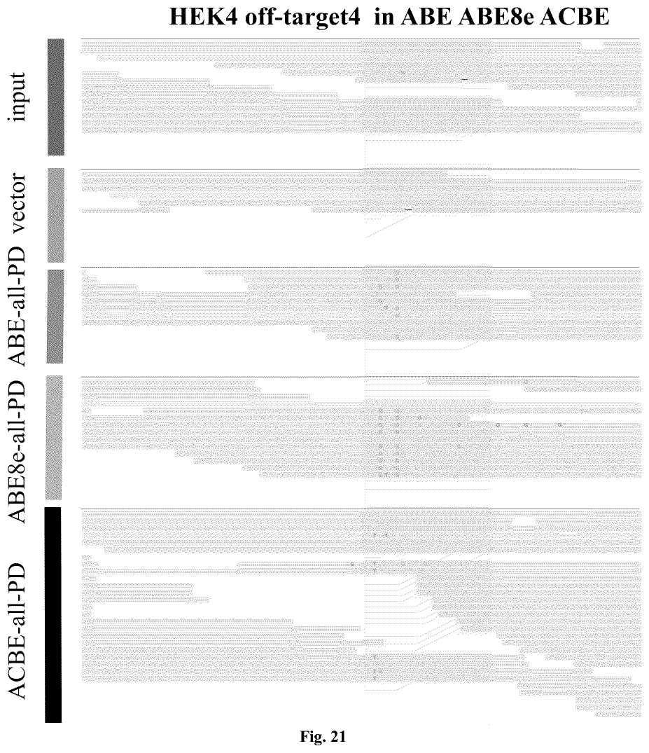

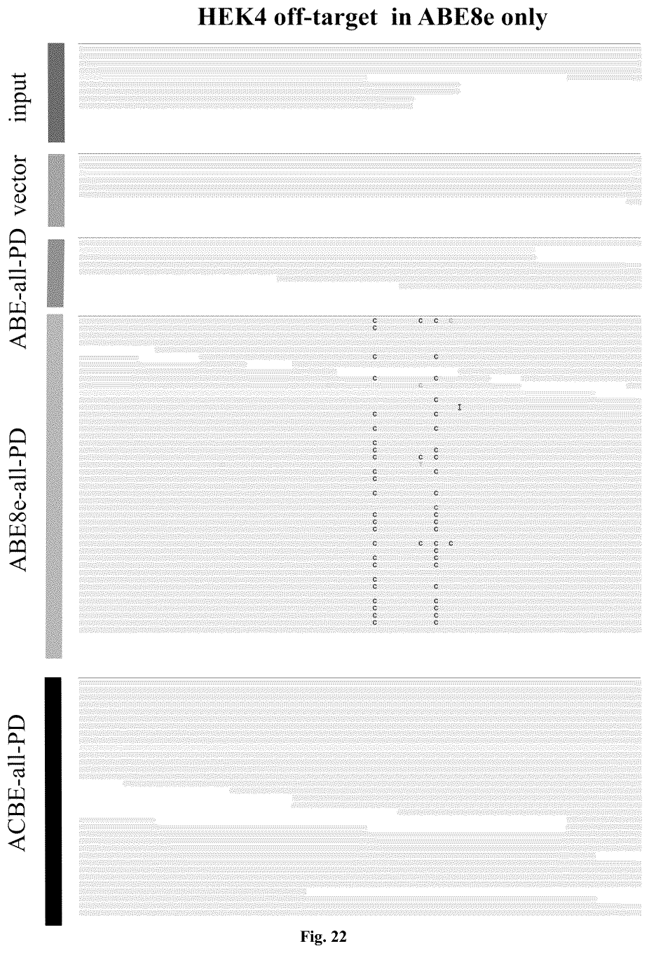

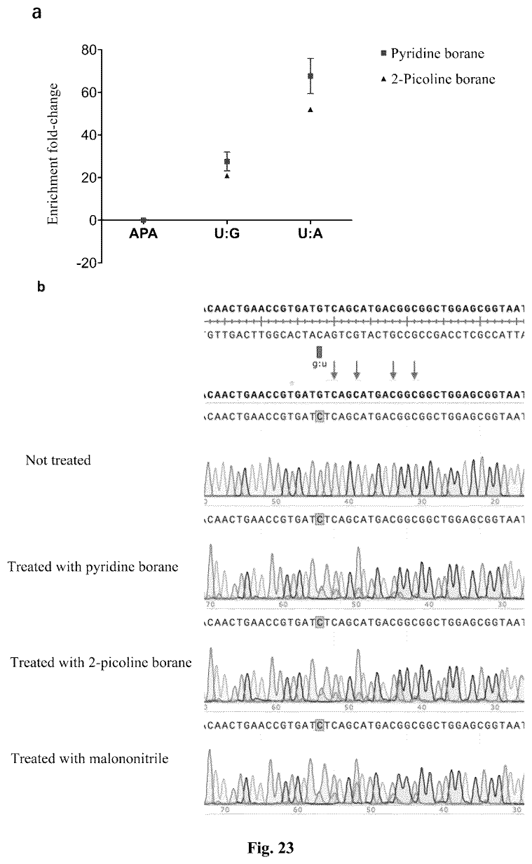

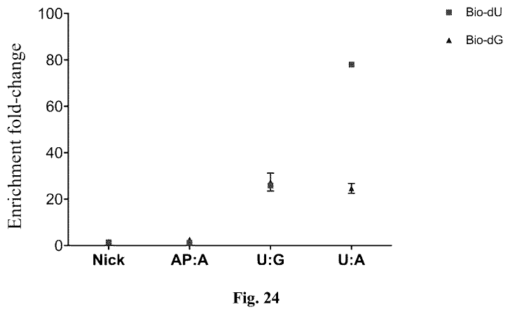

In the first step, a nucleic acid (e.g., genomic DNA or mitochondrial DNA) edited by a cytosine base editor is extracted, which comprises a base editing intermediate (e.g., DNA comprising uracil), in which the base editing intermediate is a product of the cytosine base editor editing the target nucleic acid, and comprises a first nucleic acid strand and a second nucleic acid strand; wherein, the first nucleic acid strand comprises an edited base (e.g., uracil) produced by the cytosine base editor editing the target nucleic acid. The nucleic acid is fragmented by a method such as ultrasonication to form a nucleic acid fragments of, for example, about 300 bp, and then the fragmented genomic DNA fragment is trimmed to have blunt ends through an end repair process. In certain exemplary embodiments, the end repair process comprises a process of excision of the 3′ end overhang and a process of filling-in the 5′ end overhang. In certain preferred embodiments, the end repair process can be performed using a nucleic acid polymerase having 3′ to 5′ exonucleolytic activity.

In the second step, a nucleotide (e.g., uracil deoxyribonucleotide) labeled with a first labeling molecule (e.g., biotin) and a nucleotide labeled with a second labeling molecule (e.g., 5-formylcytosine deoxyribonucleotide) are incorporated at or downstream of the position of the edited base (e.g., uracil) in the base editing intermediate through the in vitro BER (base excision repair pathway) labeling method. In some exemplary schemes, the BER labeling method comprises: using UDG (uracil-DNA glycosylase) to specifically recognize and excise uracil on the edit product produced by editing the target nucleic acid with a cytosine base editor, thereby generating an AP site; using AP endonuclease to excise the abasic sit, thereby generating a single-stranded gap; using a DNA polymerase having strand displacement activity to perform DNA strand displacement reaction along the 5′ to 3′ direction starting from the generated single-stranded gap; and using a DNA ligase to ligate a single-stranded nick in the product of the DNA strand displacement reaction. Wherein, in the DNA strand displacement reaction system, at least one nucleotide substrate (e.g., biotin-uracil ribonucleotide) labeled with a first labeling molecule (e.g., biotin) is used to replace a conventional nucleotide substrate (e.g., thymidine deoxyribonucleotide). In certain preferred embodiments, the DNA strand displacement reaction system further comprises at least one nucleotide substrate labeled with a second labeling molecule (e.g., 5-formylcytosine deoxyribonucleotide) to replace a conventional nucleotide substrate (e.g., cytosine deoxyribonucleotide). The incorporation of nucleotide labeled with the first labeling molecule (e.g., biotin-uracil deoxyribonucleotide) may allow subsequent enrichment of the nucleic acid fragment comprising the first labeling molecule by using a first binding molecule (e.g., streptavidin), wherein the first binding molecule is capable of specifically interacting with the first labeling molecule. The nucleotide labeled with the second labeling molecule is capable of complementary base pairing with different nucleotides under different conditions (e.g., before and after undergoing a treatment). For example, the nucleotide labeled with the second labeling molecule is 5-formylcytosine deoxyribonucleotide (d5fC); it is capable of complementary base pairing with guanine deoxyribonucleotide before the treatment with a compound (e.g., malononitrile, or asido-indandione), whereas, capable of complementary base pairing with adenine deoxyribonucleotide after the treatment with a compound (e.g., malononitrile, or azido-indandione), thus, the labeled product comprising d5fC can generate a C-to-T mutation signal at the position where d5fC is incorporated through subsequent chemical reactions, thereby achieving precise positioning of the position of the edited base (e.g., uracil).

In some preferred embodiments, in order to avoid false positive signals that may be caused by DNA damage or modification (e.g., SSB or AP sites) introduced endogenously or during nucleic acid manipulations, before the second step, the method further comprises performing nucleic acid repair processing on the edit product. In certain exemplary embodiments, the processing comprises: excising an AP site with an AP endonuclease to generate a single-stranded gap; using a DNA polymerase to perform DNA strand displacement reaction along the 5′ to 3′ direction starting from the generated single-chain gap or the SSB possibly existing in the nucleic acid strand; and using a DNA ligase to ligate the nick in the strand displacement reaction product. In certain preferred embodiments, the DNA polymerase has strand displacement activity.

In certain preferred embodiments, in order to avoid adverse effects of endogenous nucleotide labeled with the second labeling molecule (e.g., endogenous 5-formylcytosine deoxyribonucleotide), before undergoing the second step, the method further comprises: protecting the nucleotides labeled by the second labeling molecule that may possibly exist in the edit product. For example, before undergoing the second step, 5-formylcytosine deoxyribonucleotide that may possibly exist in the edit product can be protected with ethylhydroxylamine (EtONH₂) to prevent it from subsequent reaction with compound (e.g., malononitrile., or azido-indandione) and forming a false positive base conversion signal.

In the third step, the nucleic acid comprising the nucleotide labeled with the second labeling molecule produced in the previous step is processed to change the complementary base pairing capability of the nucleotide labeled with the second labeling molecule. In certain preferred embodiments, the nucleotide labeled with the second labeling molecule is 5-formylcytosine deoxyribonucleotide. As mentioned above, 5-formylcytosine deoxyribonucleotide treated with a compound (e.g., malononitrile, or azido-indandione) is capable of complementary base pairing with adenine deoxyribonucleotide during subsequent DNA replication process, so that, in the sequencing result of the amplified product of the processed nucleic acid, a C-to-T mutation signal will be generated at the position where the 5-formylcytosine deoxyribonucleotide is located.

In the fourth step, the DNA fragment comprising the first labeling molecule (e.g., biotin) is enriched by using a solid support (e.g., magnetic beads) coupled with the first binding molecule (e.g., streptavidin); after optionally undergoing amplification and/or library construction, it can be used for high-throughput sequencing. According to the sequencing results, the position information of the editing site in the base editing intermediate generated after editing the target nucleic acid with the cytosine base editor can be analyzed.

In some preferred embodiments, before the amplification and/or library construction of the enriched DNA fragment, the enriched DNA fragment on the solid support (e.g., magnetic beads) may undergo further treatment (e.g., alkali treatment) to remove the complementary strand of nucleic acid single strand comprising the first labeling molecule (e.g., biotin).

In certain exemplary embodiments, before the complementary strand of nucleic acid single strand comprising the first labeling molecule (e.g., biotin) is removed by alkali (e.g., NaOH) treatment, an oligonucleotide adapter is attached to an end of the enriched DNA fragment by an adapter ligation reaction, so as to facilitate the amplification or sequencing of the DNA fragment. In certain preferred embodiments, a dA tail is added to the 3′ end of the DNA fragment, and the dA tail can be used for ligation to an oligonucleotide adapter comprising a dT tail.

FIG. 2 shows the schematic diagram (a) of different model sequences used in the method of Example 1 of the present invention, and the results (b) of enriching the different model sequences by the method of Example 1 of the present invention.

FIG. 3 shows the high-throughput sequencing signal generated on model sequences by the method of Example 1 of the present invention. (a) High-throughput sequencing results of model sequences comprising dU:dG base pair. The gray dashed lines indicate the position of dU:dG base pair, and the red blocks indicate the C-to-T mutation signals; (b) The statistical calculation results of C-to-T mutation proportion at different positions on the model sequences based on the high-throughput sequencing data. Gray dashed lines indicate the position of dU:dA base pair, the solid red dots indicate the position of continuous C-to-T mutation signal, and the hollow dots indicate the position of C with signal below the background level.

FIG. 4 shows the signal generated on genomic DNA by the method of Example 1 of the present invention. (a) Signal generated at the on-target site. The upper panel indicates the signal produced at the EMX1 on-target site by the samples obtained from different editing components and different processing methods in the HEK293T cell line using the method of the present invention, and the lower panel indicates the signal produced at the VEGFA_site_2 on-target site by the samples obtained from different editing components and different processing methods in the HEK293T cell line using the method of the present invention. In the sample names, “IN” indicates the input sample, “NT” indicates the sample transfected with BE4max and non-target sgRNA, “rep1” indicates repeat 1, “rep2” indicates repeat 2; green “A” is equivalent to indicating C-to-T signal on the non-targeting strand; (b) Statistics of continuous C-to-T mutation signal generated at the genome-wide level. The left panel shows the statistics of the distance of the generated mutation signal, and the right panel shows the statistics of the number of the generated mutations; (c) The signal at a certain off-target site in the VEGFA_site_2 sample. The red block indicates the “C-to-T” mutation on the non-target strand, the red inverted triangle indicates the position actually edited by CBE, the black inverted triangle indicates the “G-to-T” SNV, and the brown shading indicates pRBS, i.e., putative sgRNA binding site; (d) Comparison between the signal of the present invention (left) and the WGS signal (right) within 4 kb before and after pRBS (dark blue) or random site (light green).

FIG. 5 shows a schematic diagram of the plasmid composition used in the comparison experiment of deleting different components in the CBE system.

FIG. 6 shows the detection results of Cas-independent off-target. (a) Examples of signals from Cas-independent off-target sites in different samples. The red “T” in the (−)sgRNA sample indicates the C-to-T signal generated by the method of the present invention, which was not observed in other samples; (b) Numbers of Cas-independent off-target sites identified in different samples; (c) Intersection of Cas-independent off-target sites identified in each All and (−)sgRNA samples; (d) The sequence motif analysis results of Cas-independent off-target sites in different samples. The 10 bp adjacent sequences on both sides of each site (referring to hg38 genome) were extracted and subjected to sequence analysis by WebLogo software; (e) The Cas-independent off-target sites identified by the method of the present invention were enriched and appeared in the genome transcription active regions; (f) The Cas-independent off-target sites identified by the present invention were more concentrated in the highly expressed gene regions. All P values were calculated by one-sided Student's t-test.

FIG. 7 shows the detection results of Cas-dependent off-targets. (a) Examples of signals from Cas-dependent off-target sites in different samples. In the enlarged IGV (Integrative Genomics Viewer) diagram on the right, the green block indicates the “G-to-A” mutation, which is equivalent to the “C-to-T” mutation on the non-target chain: (b) Cas-dependent off-target sites were identified in two biological replicates of “VEGFA_site_2 -ALL”. Under the very strict bioinformatics analysis and identification rules (cutoff), 384 loci were judged to be repeated (orange dots; including on-target), but in fact the remaining rep-only dots (blue dots) signal intensity was not low in both samples; (c) Comparison of signals of the present invention at all Cas-dependent off-target sites at the whole genome level in different samples. The signals of endogenous dU modification (gray dots) naturally existing in the cell basically remained unchanged in the diagonal position, while the signal intensity of on-target sites (red dots) and Cas-dependent off-target sites (orange dots) varied with the removed components.

FIG. 8 shows the comparison between the signal intensity detected by the method of Example 1 of the present invention and the results of targeted deep sequencing. ρ indicates the Spearman correlation coefficient. Note: All shown in the figure are the verification data of Cas-dependent off-target sites.

FIG. 9) shows the verification of two examples of Cas-dependent off-target detected by the method of the present invention by targeted deep sequencing. (a) The real editing efficiency at the “VEGFA_site_2 pRBS-237” off-target site in different samples; (b) The real editing efficiency at the “VEGFA_site_2 pRBS-67” off-target site in different samples.

FIG. 10 shows the distribution of “EMX1”, “VEGFA_site_2” and “HEK293 site_4” sgRNA on-target editing sites and Cas-dependent off-target editing sites detected at the genome-wide level by the method of the present invention on each chromosome. On-target editing sites and Cas-dependent off-target editing sites are indicated by red squares and blue circles, respectively.

FIG. 11 shows a Venn diagram of comparing Cas-dependent off-target sites detected by the method of Example 1 of the present invention with GUIDE-seq (a) and Digenome-seq (b), respectively.

FIG. 12 shows the re-evaluation results of the specificity of the CBE optimization tool YE1-BE4max using the method of the present invention. (a) Comparison of detection signals of all Cas-dependent off-target sites at the genome-wide level in YE1-BE4max (vertical axis) and WT-BE4max (horizontal axis) samples; (b) Editing efficiencies of YE1-BE4max and WT-BE4max at different sites. Red triangles indicate where substantial off-target edits remain.

FIG. 13 shows the Cas-dependent off-target caused by LbCpf1-BE at the genome-wide level for the “RUNX1” and “DYRK1A” sites detected by the method of Example 1 of the present invention. The abscissa and ordinate are the signal intensities identified by the present invention in two biological replicate samples.

FIG. 14 shows the examples of TALE-array sequence (TAS) dependent off-target (a) and TALE-array sequence (TAS) independent off-target (b) caused by CRISPR-free DdCBE tool. asdetected by the method of Example 1 of the present invention. The upper panel shows an enlarged IGV (Integrative Genomics Viewer) diagram, the red block indicates the “C-to-T” mutation, and the green block indicates the “G-to-A” mutation, which is equivalent to the “C-to-T” mutation on the complementary chain; the middle panel shows the mCherry of negative control sample; the lower panel shows the sequencing result of the off-target sites detected by the method of the present invention by the targeted deep sequencing method for verification.

FIG. 15 shows an exemplary scheme 2 for detecting an editing site of a base editor using the method of the present invention, wherein the base editor is an adenine base editor.

First, in the first step, a nucleic acid (e.g., genomic DNA) edited by an adenine base editor is extracted, which comprises a base editing intermediate (e.g., a DNA comprising inosine), in which the base editing intermediate is a product of the adenine base editor editing the target nucleic acid, and comprises a first nucleic acid strand and a second nucleic acid strand; wherein the first nucleic acid strand comprises an edited base (e.g., inosine) generated by the adenine base editor editing the target nucleic acid. The nucleic acid is fragmented by a method such as ultrasonication to form a nucleic acid fragment of, for example, about 300 bp, and then the fragmented genomic DNA fragment is trimmed to blunt ends through an end repair process. In certain exemplary embodiments, the end repair process comprises a process of excision of the 3′ end overhang and a process of filling-in the 5′ end overhang. In certain preferred embodiments, the end repair process can be performed using a nucleic acid polymerase having 3′ to 5′ exonucleolytic activity.

In the second step, a nucleotide (e.g., uracil deoxyribonucleotide) labeled with a first labeling molecule (e.g., biotin) is incorporated downstream of the position where the edited base (e.g., inosine) is located in the base editing intermediate through an in vitro labeling method. In some exemplary schemes, the labeling method comprises: using an endonuclease Endo V to specifically recognize inosine in the base editing intermediate, and cleaving the second phosphodiester bond at the 3′ end of the inosine deoxyribonucleotide to form a single-strand gap; using a DNA polymerase with strand displacement activity to carry out DNA strand displacement reaction along the 5′ to 3′ direction starting from the generated single-strand gap; using a DNA ligase to ligate the single-strand nick in the product of the DNA strand displacement reaction. Wherein, in the DNA strand displacement reaction system. at least one nucleotide substrate labeled with a first labeling molecule (e.g., biotin), e.g., biotin-uracil ribonucleotide, is used to replace a conventional nucleotide substrate (e.g., thymidine deoxyribonucleotide). The incorporation of the nucleotide labeled with the first labeling molecule (e.g., biotin-uracil deoxyribonucleotide) may allow subsequent enrichment of the DNA fragment comprising the first labeling molecule by using the first binding molecule (e.g., streptavidin). The edited base (e.g., inosine) comprised in the base editing intermediate will be able to complementary pair with cytosine during subsequent DNA replication and sequencing process, so that in the sequencing results of the labeled product, A-to-G mutation signal will be generated at the position of inosine. Thus, by detecting the presence of the mutation signal, precise positioning of the edited base (e.g., inosine) can be achieved.

In some preferred embodiments, in order to avoid false positive signals that may be brought about by DNA damage (e.g., SSB) introduced endogenously or during nucleic acid manipulation, before undergoing the second step, the method further comprises, allowing the edit product to undergo nucleic acid repair processing. In certain exemplary embodiments, the processing comprises: using a DNA polymerase to carry out a DNA strand displacement reaction along the 5′ to 3′ direction starting from the SSB; and using a DNA ligase to ligate the nick in the displacement reaction product. In certain preferred embodiments, the DNA polymerase has strand displacement activity.

In the third step, the DNA fragment comprising the first labeling molecule (e.g., biotin) is enriched by using a solid support (e.g., magnetic beads) coupled with the first binding molecule (e.g., streptavidin); optionally, after undergoing amplification and/or library construction, it can be used for high-throughput sequencing. According to the sequencing results, the position information of the editing site in the base editing intermediate (e.g., a DNA comprising inosine) generated by the adenine base editor editing the target nucleic acid can be analyzed.

In some preferred embodiments, before the amplification and/or library construction of the enriched DNA fragment, the enriched DNA fragment on the solid support (e.g., magnetic beads) can further undergo a treatment (e.g., alkali treatment) to remove the complementary strand of the nucleic acid single strand comprising the first labeling molecule (e.g., biotin).

In certain exemplary embodiments, before the complementary strand of the nucleic acid single strand comprising the first labeling molecule (e.g., biotin) is removed by alkali (e.g., NaOH) treatment, an oligonucleotide adapter is attached to an end of the enriched DNA fragment through an adapter ligation reaction so as to facilitate the amplification or sequencing of the DNA fragment. In certain preferred embodiments, a dA tail is added to the 3′ end of the DNA fragment, and the dA tail can be used to ligate to an oligonucleotide comprising a dT tail.

FIG. 16 shows the enrichment results of different model sequences by the method of Example 2 of the present invention.

FIG. 17 shows the high-throughput sequencing results of ABE at the on-target site of HEK293_site_4 sgRNA (referred to as HEK4) for each sample group. The shade indicates the sequence position of on-target, wherein “G” is the A-to-G mutation signal.

FIG. 18 shows the high-throughput sequencing results of ABE at the off-target site (off-target 4) of HEK4 for each sample group. The shade indicates the possible sgRNA binding sequence position, wherein “G” is the A-to-G mutation signal.

FIG. 19 shows the targeted deep sequencing verification results of ABE at the off-target site (off-target 4) of HEK4. The first two rows of sequences are the on-target sequence and the sequence of the off-target site; and the last six rows represent the proportions of A, G, C, T bases as well as insertions and deletions, respectively.

FIG. 20 shows the high-throughput sequencing results of HEK4 sgRNA at the on-target sites in ABE, ABE8e and ACBE systems. Orange G represents A-to-G mutation signal; and red T represents C-to-T mutation signal.

FIG. 21 shows the high-throughput sequencing results of HEK4 sgRNA at the off-target site (off-target4) in ABE, ABE8e and ACBE systems. Orange G represents A-to-G mutation signal; and red T represents C-to-T mutation signal.

FIG. 22 shows the high-throughput sequencing results at the ABE8e-only off-target sites for ABE, ABE8e and ACBE systems. Blue C represents the T-to-C mutation signal, i.e., represents the A-to-G mutation signal on its complementary strand.

FIG. 23 shows the characterization results of the present invention on the spike-in sequence after replacing the malononitrile labeling step with other 5fC labeling methods (pyridine borane labeling reaction or 2-picoline borane labeling reaction). Among them, (FIG. 23a) shows the qPCR enrichment results of the present invention for different model sequences (AP:dA, dU:dA or dU:dG) after replacing with the chemical labeling method of pyridine borane compound (pyridine borane or 2-picoline borane); (FIG. 23b) shows the Sanger sequencing results of the present invention for the model sequence comprising dU:dG base pair after replacing with the chemical labeling method of pyridine borane compound (pyridine borane or 2-picoline borane). Red arrows indicate the C-to-T mutation signals triggered by the chemical labeling.

FIG. 24 shows the qPCR enrichment results of the present invention for different model sequences (Nick, AP:dA, dU:dA or dU:dG) after replacing the Biotin-dU in the present invention with Biotin-dG.

### Sequence Information

Information of the sequences involved in the present invention is provided in Table 1 below:

TABLE 1

| SEQ | |
|---|---|
| ID | |
| NO: | sequence description (5′-3′) |
| 1 | Control model sequence |
| | AACTGATTGCCCGTCTCCGCTCGCTGGGTGAACAACTGAACCGTGATGT |
| | CAGCATGACGTTATCTGGCGGTGGAGATGGCTCCGTGTGGCAGAGCTG |
| | AAAGAGGAGCTTGATGACACGTAATGCTTGCGTGGCAAAC |
| 2 | dU:dA-1 model sequence |
| | AACTGATTGCCCGTCTCCGCTCGCTGGGTGAACAACTGAACCGTGAT(dU) |
| | TCAGCATGACGGCGGTAAGCACGAACTCAGGCTCCGTGTGGCAGAGCT |
| | GAAAGAGGAGCTTGATGACACGGGAAATACCGTGGTGTGGC |
| 3 | dU:dA-2 model sequence |
| | AACTGATTGCCCGTCTCCGCTCGCTGGGTGAACAACTGAACCGTGAT(dU) |
| | TCAGCATGACGCATGAGTGCCCTCAGCAGTAGCTCCGTGTGGCAGAGC |
| | TGAAAGAGGAGCTTGATGACACGTCCAACCTTTAGGAGCCATG |
| 4 | AP:dA model sequence |
| | AACTGATTGCCCGTCTCCGCTCGCTGGGTGAACAACTGAACCGTGAT(A |
| | P)TCAGCATGACGCATGAGTGCCCTCAGCAGTAGCTCCGTGTGGCAGAG |
| | CTGAAAGAGGAGCTTGATGACACGTCCAACCTTTAGGAGCCATG |
| 5 | dU:dG model sequence |
| | AACTGATTGCCCGTCTCCGCTCGCTGGGTGAACAACTGAACCGTGAT(dU) |
| | TCAGCATGACGGCGGCTGGAGCGGTAATTTTGCTCCGTGTGGCAGAGC |
| | TGAAAGAGGAGCTTGATGACACGTAATGACGTTGCCAGCCAGT |
| 6 | d5fC:dG model sequence |
| | CATGAGTGCCCTCAGCAGTAAGTAACTGACCAGATCTCTCGTGCCTCTT |
| | GAGGCTACTGAGTTATCCAACCTTTAGGAGCCATGCATCGATAGCATCC |
| | G(d5fC)CACAGGCAGTGAGGCTACTGAGTCATGCACGCAGAAAGAAATA |
| | GC |
| 7 | Forward single-strand sequence of Y-type adapter |
| | P-GATCGGAAGAGCACACGTCTGAACTCCAGTC(AMN) |
| 8 | Reverse single-strand sequence of Y-type adapter |
| | ACACTCTTTCCCTACACGACGCTCTTCCGATCT |
| 9 | Universal Primer sequence |
| | AATGATACGGCGACCACCGAGATCTACACTCTTTCCCTACACGACGCTC |
| | TTCCGATCT |
| 10 | Index Primer sequence |
| | CAAGCAGAAGACGGCATACGAGATNNNNNNGTGACTGGAGTTCAGACG |
| | TGTGCTCTTCCGATCT |
| 11 | qPCR Primer-1 for control model sequence |
| | TTATCTGGCGGTGGAGATG |
| 12 | qPCR Primer-2 for control model sequence |
| | GTTTGCCACGCAAGCATTA |
| 13 | qPCR Primer-1 for dU:dA-1 model sequence |
| | GCGGTAAGCACGAACTCAG |
| 14 | qPCR Primer-2 for dU:dA-1 model sequence |
| | GCCACACCACGGTATTTCC |
| 15 | qPCR Primer-1 for dU:dA-2 model sequence |
| | CATGAGTGCCCTCAGCAGTA |
| 16 | qPCR Primer-2 for dU:dA-2 model sequence |
| | CATGGCTCCTAAAGGTTGGA |
| 17 | qPCR Primer-1 for AP:dA model sequence |
| | CATGAGTGCCCTCAGCAGTA |
| 18 | qPCR Primer-2 for AP:dA model sequence |
| | CATGGCTCCTAAAGGTTGGA |
| 19 | qPCR Primer-1 for dU:dG model sequence |
| | GCGGCTGGAGCGGTAATTTT |
| 20 | qPCR Primer-2 for dU:dG model sequence |
| | ACTGGCTGGCAACGTCATTA |
| 21 | qPCR Primer-1 for d5fC:dG model sequence |
| | CATGAGTGCCCTCAGCAGTA |
| 22 | qPCR Primer-2 for d5fC:dG model sequence |
| | CATGGCTCCTAAAGGTTGGA |
| 23 | VEGFA_site_2 sgRNA target site sequence |
| | GACCCCCTCCACCCCGCCTC |
| 24 | HEK293 site_4 sgRNA target site sequence |
| | GGCACTGCGGCTGGAGGTGG |
| 25 | EMX1 sgRNA target site sequence |
| | GAGTCCGAGCAGAAGAAGAA |
| 26 | RNF2 sgRNA target site sequence |
| | GTCATCTTAGTCATTACCTG |
| 27 | RUNX1 sgRNA target site sequence |
| | TCCCCTCTGCTGGATACCTC |
| 28 | dI:dC model sequence |
| | AACTGATTGCCCGTCTCCGCTCGCTGGGTGAACAACTGAACCGTGATTTCA |
| | (dI)CATGACGAATGTGGATGCCGCAGTTGGCTCCGTGTGGCAGAGCTGAAA |
| | GAGGAGCTTGATGACACGCAACCGGGACATCACGGAT |
| 29 | dI:dT model sequence |
| | AACTGATTGCCCGTCTCCGCTCGCTGGGTGAACAACTGA(dI)CCGTGATGTC |
| | AGCATGACGCTACGCAAACTGGCTGTCAAGCTCCGTGTGGCAGAGCTGAA |
| | AGAGGAGCTTGATGACACGTCATGGACGCTACCTCACAG |
| 30 | Nick model sequence |
| | AACTGATTGCCCGTCTCCGCTCGCTGGGTGAACAACTGAACCGTGATAGTC |
| | AGCATGACGAGGCCAACATACATGCCTTCGCTCCGTGTGGCAGAGCTGAA |
| | AGAGGAGCTTGATGACACGGAATGGCAGAGTCAAGGAGC |
| 31 | qPCR Primer-1 for dI:dC model sequence |
| | AATGTGGATGCCGCAGTTG |
| 32 | qPCR Primer-2 for dI:dC model sequence |
| | ATCCGTGATGTCCCGGTTG |
| 33 | qPCR Primer-1 for dI:dT model sequence |
| | CTACGCAAACTGGCTGTCAA |
| 34 | qPCR Primer-2 for dI:dT model sequence |
| | CTGTGAGGTAGCGTCCATGA |
| 35 | qPCR Primer-1 for Nick model sequence |
| | AGGCCAACATACATGCCTTC |
| 36 | qPCR Primer-2 for Nick model sequence |
| | GCTCCTTGACTCTGCCATTC |
| 37 | RUNX1 crRNA target site sequence |
| | TTCTCCCCTCTGCTGGATACCTC |
| 38 | DYRKIA crRNA target site sequence |
| | GAAGCACATCAAGGACATTCTAA |

Note:
The symbol “\hat{ }” indicates Nick site; N = A, T, G, or C; the symbol “P” indicates phosphorylation modification; “AMN” indicates C7 Aminolinker blocking.

### Specific Models for Carrying Out the Present Invention

The present invention will now be described with reference to the following examples, which are intended to illustrate the present invention, but not to limit it.

In the examples, those without specific conditions were carried out according to the conventional conditions or the conditions recommended by the manufacturer. The reagents or instruments used not indicated by the manufacturers were all commercially available conventional products. Those skilled in the art understand that the examples describe the present invention by way of example and are not intended to limit the protection scope of the present application.

### Example 1: Detection of CBE Edited Site

### Experimental Method

1. DNA fragmentation

Genomic DNA was extracted from living cells HEK293T (purchased from ATCC, Catalog No.: CRL-11268) or MCF7 (purchased from ATCC, Catalog No.: HTB-22) that had been transfected with the CBE system. The method of transfecting cells with CBE system was referred to (Xiao Wang, et al. Nature biotechnology 36, 946-949, doi: 10.1038/nbt.4198 (2018)), and the method of extracting genomic DNA from cells was referred to the kit manual (purchased from JiangSu CoWin Biotech (CWBIO), Catalog No.: CW2298M).

The extracted genomic DNA was fragmented into fragments with a length of about 300 bp by a Covaris ME220 ultrasonic breaker, and then recovered by a DNA Clean & Concentrator-5 Kit (purchased from VISTECH, Catalog No.: DC2005).

### 2. DNA Fragment End Repair

The DNA fragmented according to the above step 1 still had some nicks and overhangs. If they were not repaired, they would be labeled with biotin in the subsequent labeling reaction, thereby resulting in false positives. Therefore, in this step, NEB end repair module (Catalog No.: E6050) and E.coli DNA ligase (purchased from NEB, Catalog No.: M0205) were used to repair the genomic DNA damage possibly caused by the fragmentation process.

The reaction system was prepared according to Table 2:

TABLE 2
End repair reaction system

| Component | Total system (100 μL) |
|---|---|
| DNA fragmented in Step 1 | 79 μL (~5 ug) |
| Spike-in model sequence (SEQ ID NO: 1 | 2 μL (5-10 pg of |
| and one or more of SEQ ID NOs: 2-6) | each model sequence) |
| End Repair Reaction Buffer | 10 μL |
| End Repair Enzyme Mix | 5 μL |
| 50 mM NAD⁺ | 2 μL |
| E. coli DNA ligase | 2 μL |

The above reaction system was mixed on ice, then reacted at 20° C. for 30 min, recovered with 2.0×AMPure XP beads (purchased from Beckman Coulter, Catalog No.: NC9933872), and eluted with ddH₂O.

### 3. EtONH₂ Protection

The end-repaired DNA fragment prepared in step 2 was incubated in 80 μL of 100 mM MES buffer (pH 5.0) containing 10 mM EtONH₂ at 37° C. for 6 h, so that the naturally occurring d5fC modification in the cells was protected and could not react with subsequently used malononitrile to produce false positive. Subsequently, the DNA after the reaction was recovered using the DNA Clean & Concentrator-5 Kit.

### 4. Addition of dA Tail

The DNA fragment obtained in step 3 was added at 3′ end with a dA to facilitate the subsequent ligation to a sequencing adapter (Adaptor) using the A/T complementarity rule.

The reaction system was prepared according to Table 3:

TABLE 3
Reaction system for addition of dA tail

| Component | Total system (50 μL) |
|---|---|
| DNA protected in step 3 | 42 μL (~3 ug) |
| dA-tailing Reaction Buffer (purchased from | 5 μL |
| NEB, Catalog No.: E6053) | |
| Klenow Fragment (3′-5′ exo⁻) (purchased | 3 μL |
| from NEB, Catalog No.: E6053) | |

The above reaction system was mixed on ice, then reacted at 37° C. for 30 min, recovered with 2.0×AMPure XP beads, and eluted with ddH₂O.

### 5. DNA Damage Repair

The purpose of this step was to repair and remove the naturally occurring AP sites, SSB, Nick and other DNA modifications or damages that might generate false positive signals before dU labeling.

The reaction system was prepared according to Table 4:

TABLE 4
Reaction system for damage repair

| Component | Total system (50 μL) |
|---|---|
| DNA prepared in step 4 | 38 μL (~2.7 ug) |
| NEBuffer 3.0 (purchased from NEB, Catalog | 5 μL |
| No.: B7003S) | |
| 50 mM NAD⁺ | 1 μL |
| 2.5 mM dNTPs | 1 μL |
| Endo IV (purchased from NEB, Catalog | 2 μL |
| No. M0304) | |
| Bst full-length polymerase (purchased from NEB, | 1 μL |
| Catalog No.: M0328) | |
| Taq DNA ligase (purchased from NEB, Catalog | 2 μL |
| No.: M0208) | |

The above reaction system was mixed well, firstly reacted at 37° C. for 60 min, subsequently at 45° C. for 60 min, then recovered with 2.0×AMPure XP beads, and eluted with ddH₂O.

### 6. In Vitro BER Labeling Assay

0.5 μL of the DNA obtained in the above step 5 was taken and added with 0.5 μL of ddH₂O as Input, and the remaining sample was subjected to the labeling reaction as follows.

Reaction system was prepared according to table 5:

TABLE 5
In vitro labeling reaction system

| Component | Total system (50 μL) | |
|---|---|---|
| DNA prepared in Step 5 | 37 μL (~2.5 ug) | |
| NEBuffer 3.0 | 5 | μL |
| 50 mM NAD⁺ | 1 | μL |
| 5 μM dATP/dGTP/Biotin-dUTP/20 μM d5fCTP | 2 | μL |
| UDG (purchased from NEB, Catalog No.: | 1 | μL |
| M0280) | | |
| Endo IV | 1.5 | μL |
| Bst full-length polymerase | 0.8 | μL |
| Taq DNA ligase | 1.7 | μL |

The above reaction system was mixed well, then reacted at 37° C. for 40 min, recovered with 2.0×AMPure XP beads, and eluted with ddH₂O.

### 7. Malononitrile Reaction

The DNA recovered from step 6 above was placed in 50 mM Tris-HCl (pH 7.0) containing 75 mM malononitrile, and reacted in a mixer at 37° C. with a rotation speed of 800 rpm for 20 h. It was then recovered again by 2×AMPure XP beads and eluted with ddH₂O.

### 8. Enrichment of Fragment

Each PD (pull down) sample corresponded to 10 μL of Streptavidin C1 beads (purchased from Invitrogen, Catalog No.: 65002). The beads in a sufficient amount were taken and washed three times with 1×B&W buffer (5 mM Tris-HCl (pH 7.5), 1 M NaCl, 0.5 mM EDTA, 0.05% Tween-20), then resuspended with 40 μL of 2×B&W buffer, then added with an equal volume of sample DNA treated in step 7 above, mixed well, and incubated under rotation at room temperature for 1 h. The magnetic beads were then washed three times with 1×B&W buffer, and once with 10 mM Tris-HCl (pH 8.0), under rotation at room temperature for 5 min each time. Finally, the Tris-HCl liquid was sucked out on a magnetic stand, and the remaining magnetic beads bound with DNA fragment (about 1 μL in volume) were used for the adapter ligation reaction.

### 9. Adapter Ligation

1) Adapter stock solution (30 μM) was diluted to 1.5 μM with 10 mM Tris-HCl on ice. The Y-type adapter used was obtained by annealing two single-strand sequences, wherein the forward single-strand was phosphorylated at 5′ end, and blocked at 3′ end by a C7 Aminolinker, the sequence of which was shown in SEQ ID NO:7, and the reverse single-stranded sequence was shown in SEQ ID NO:8.

2) NEBNext® Quick Ligation Module (purchased from NEB, Catalog No.: E6056) was used to perform adapter ligation reaction on the Input sample (aqueous solution) retained in step 6 and the PD sample (connected to magnetic beads) obtained in step 8 above.

The reaction system was prepared according to table 6:

TABLE 6
Adapter ligation reaction system

| Component | Total system (25 μL) | |
|---|---|---|
| ddH₂O | 14 | μL |
| NEB Quick Ligation Buffer | 5 | μL |
| 1.5 μM Y-type adaptor | 2.5 | μL |
| Quick T4 DNA Ligase | 2.5 | μL |
| PD or Input sample DNA | 1 | μL |

For the adapter ligation reaction of the PD sample: the above reaction system was mixed, then reacted at about 20° C. for 1 h under rotation (to avoid magnetic beads sedimentation), added with 50μl of 1×B&W buffer, incubated continuously at room temperature for 1 h (to allow a small amount of DNA fragment that was separated during the ligation process to ligate to the magnetic beads again), and then underwent the next reaction;

For the adapter ligation reaction of the Input sample: the above reaction system was mixed, then placed in a PCR instrument and reacted at 20° C. for 40 min, and subjected to recovery and retention with 1×AMPure XP beads to remove the adapters that had not been successfully ligated.

### 10. NaOH Treatment

The PD sample on the magnetic beads obtained in the above step 9 was washed 3 times with 1×B&W buffer, and then washed once with 1×SSC buffer, in which the magnetic beads were firstly shaken by gently overturning and then rotated at room temperature for 5 min for each time. The supernatant was then discarded, the remaining magnetic beads were resuspended in 20 μl of 0.15M NaOH solution and incubated at room temperature under rotation for 10 min, then washed with 1×SSC buffer, 10 mM Tris-HCl (pH 8.0) once in succession. Finally, the magnetic beads were treated with ddH₂O at 95° C. for 3 min, and the DNA library on the magnetic beads was eluted for the next PCR amplification step.

### 11. Library Amplification

1) Because the amplification process of high-fidelity DNA polymerase was easily interrupted by Biotin-dU and malononitrile-labeled d5fC, the library was first amplified with Mighty Amp DNA Polymerase (purchased from TaKaRa, Catalog No.: R076A) that had a slightly lower fidelity.

The reaction system was prepared according to table 7:

TABLE 7
Mighty Amp amplification system

| Component | Total system (50 μL) | |
|---|---|---|
| PD sample obtained in Step 10 or Input DNA | 22 | μL |
| sample obtained in Step 9 | | |
| 2 × Mighty Amp Buffer Ver.3 (Mg²⁺, dNTP plus) | 25 | μL |
| 20 μM Universal Primer (SEQ ID NO: 9) | 1 | μL |
| 20 μM Index Primer (SEQ ID NO: 10) | 1 | μL |
| Mighty Amp DNA Polymerase Ver. 3 | 1 | μL |

The above reaction system was mixed and then underwent PCR reaction. The program was: 98° C. for 30 s; 98° C. for 10 s, 65° C. for 90 s (2 cycles); 72° C. for 5 min. DNA after the reaction was recovered using DNA Clean & Concentrator-5 Kit (VISTECH).

2) Subsequent amplification was performed using a high-fidelity DNA polymerase to ensure low overall sequencing noise background.

The reaction system was prepared according to table 8:

TABLE 8
High-fidelity amplification system

| Component | Total (50 μL) | |
|---|---|---|
| DNA sample obtained in the process 1) of step 11 | 22.5 | μL |
| Q5 ® Hot Start High-Fidelity 2X Mix (purchased | 25 | μL |
| from NEB, Catalog No.: M0494) | | |
| 20 μM Universal Primer (SEQ ID NO: 9) | 1.25 | μL |
| 20 μM Index Primer (SEQ ID NO: 10) | 1.25 | μL |

The above reaction system was mixed and then underwent PCR reaction. The program was: 98° C. for 30 s; 98° C. for 10 s, 65° C. for 90 s (8-9 cycles for PD sample; 6-7 cycles for Input sample); 72° C. for 5 min. The PCR product was recovered with 0.9×AMPure XP beads and eluted with ddH₂O.

### 12. Quality Check of Library

Qubit2.0 precision spectrophotometer was used to measure the library concentration;

Fragment Analyzer 12 automatic capillary electrophoresis instrument was used to check the distribution of library fragments:

qPCR was used to relatively quantify the model sequence and calculate the enrichment fold-change. The primers used in qPCR were shown in SEQ ID NOs:11-22. The data processing adopted the 2^{-ΔΔCt} method. The enrichment fold-change was a fold-change of the relative amount of the spike-in DNA molecule that comprised a specific type of modification in the PD sample (using the Control model sequence as a reference) compared to the corresponding Input sample, and based on this fold-change, the enrichment of this batch of experiments could be evaluated;

The model sequence underwent full-length PCR amplification, the obtained PCR product underwent Sanger sequencing, and the labeling status of this batch of experiments could be evaluated through the sequencing results;

Finally, the resulting library was delivered to the Illumina Hiseq X-ten platform for paired-end sequencing (read length 150 bp).

### Processing and Analysis of Sequencing Data:

### 1. Back-Pasting and Filtering of Data in the Present Invention

After the data was downloaded from machine, the cutadapt (version 1.18) software was firstly used to remove the sequencing adapters from the sequencing reads (reads) in the FASTQ file of the sequencing results. The specific command parameters were: cutadapt—times 1 −e 0.1 −O 3—quality-cutoff 25 −m 50. After removing the adapters, considering that the sequencing results of the present invention could contain mutations from C to T, the Bismark (version 0.22.3) software was firstly used to paste the sequencing reads from which the sequencing adapters had been removed to the reference genome (version number was hg38). Sequencing reads that did not align successfully or whose alignment quality MAQP was lower than 20 were re-extracted and then re-aligned using BWA MEM (version 0.7.17). Finally, the sequencing data after two alignments and merging were screened again, and only alignment results with an alignment quality MAPQ greater than 20, that was, an alignment error rate less than 1%, were retained for downstream analysis. Next, the screened high-quality alignment results underwent a deduplication processing that was carried out with the Picard MarkDuplicates command (version 1.9). The main purpose of this step was to remove the molecular redundancy caused by amplification during the library construction process. After the above steps, the genome back-pasting results (BAM format files) were obtained for downstream analysis.

### 2. Preliminary Identification of Signals of the Present Invention

The samtools mpileup −q 20 −Q 20 command (version 1.9) was used to convert the BAM files to mpileup files. Then, the parse-mpileup command and the bmat2pmat command in the written software tool (see, for example, https://github.com/menghaowei/Detect-seq) were used to generate pmat files. Then the pmat-merge command was used to scan and organize all the concatenated C to T mutation signals in the whole genome and record them into mpmat format files. Finally, the mpmat-select command was used for screening, thereby obtaining the preliminary sequencing signals of the present invention.

### 3. Identification of Enrichment Signals of the Present Invention

After obtaining the preliminary sequencing signals of the present invention, it was necessary to perform enrichment detection on these candidate regions. First, the find-significant-mpmat command in the software tool was used to perform a statistical test on the candidate regions, and the results of the statistical test were corrected by the BH method to obtain a false discovery rate (FDR). Finally, it was considered that the regions with the FDR less than 0.01, the enrichment fold-change of the treatment group greater than 2 after normalization compared with the control group, the reads with mutation signal in the sample of the control group less than 3, and the sequencing reads with mutation signal in the treatment group sample not less than 5, were the final identification regions of the present invention.

### 4. Removal of Endogenous Deoxyuracil Site

In the enrichment test, the experimental group and the control group were set as the sample that was only transfected with empty plasmid and processed by the enrichment library construction process described in this method, and the sample that was not processed by the enrichment library construction process described in this method respectively, and thus, the position information of endogenous deoxyuracil could be obtained. In order to ensure that this identification method had a lower false negative rate, a looser threshold was used in this step: FDR was less than 0.05, and the normalized enrichment fold-change of the experimental group compared with the control group was greater than 1.5.

5. Alignment of Off-Target Site Gene Sequence and sgRNA Sequence

In the enrichment signal region where endogenous dU was removed as identified in the above steps, the binding site of sgRNA/crRNA could be deduced by sequence alignment. This deduced sgRNA/crRNA binding site was called pRBS (putative sgRNA/crRNA binding site). When performing sequence alignment between sgRNA/crRNA and the enrichment signal region, an improved semi-global alignment method was used. For sgRNA, the PAM sequence (NAG/NGG) was searched first in the region, and then for the found PAM site, the sequence of 30 nt in the 5′ direction of the PAM was extracted to perform semi-global double-sequence alignment with the sgRNA, and the optimal result reported in the alignment was pRBS; for crRNA, the PAM sequence (TTTV, V=A/C/G) was searched first in the region, and then at the searched PAM site, the sequence of 30 nt in the 3′ direction of PAM was extracted to perform half-global double sequence alignment with the crRNA, and the optimal result reported in the alignment was the pRBS of crRNA. In the above process, if no PAM was found in the region, the sgRNA/crRNA was directly used to perform the semi-global alignment with the sequence of the region, and the optimal result of the alignment was the pRBS of sgRNA/crRNA. The alignment parameters used for this step were: match +5: mismatch −4: open gap −24: gap extension −8. The alignment program for this step was comprised in the mpmat-to-art command in the Detect-seq software toolbox.

### Experimental Results:

1. Specific Labeling and Enrichment of dU-Comprising Model Sequence

In order to prove the specificity and efficiency of the method of the present invention, the model sequences and control sequences (SEQ ID NOs: 1-6) comprising different modified bases shown in FIG. 2a were incorporated into the genomic DNA after fragmentation, and then the library was constructed according to the above experimental method. Finally, the ratio changes of different model sequences in samples before and after pull-down were calculated and compared by fluorescent quantitative PCR technology (relative quantification was performed with the control sequence without any modification (Control model sequence as shown in SEQ ID NO: 1)), and the enrichment fold-changes of different model sequences in samples before and after pull-down were calculated. The enrichment fold-changes were shown in FIG. 2b. It could be seen from the figure that for the model sequences comprising single dU:dA and dU:dG base pairs, the method provided by the present invention could enrich them by about 60 times and about 30 times, respectively; whereas for the model sequences comprising AP sites, d5fC, they were almost not enriched at all. This showed that the method provided by the present invention could specifically enrich dU-comprising DNA fragments.

On the other hand, according to the principle of the present invention, a plurality of d5fCTPs could be continuously incorporated at the 3′ end of the position of dU with a certain probability, so that continuous C-to-T mutations would be generated thereafter to achieve signal amplification for detection purposes. From the results of Sanger sequencing and high-throughput sequencing (FIG. 3), we had indeed observed continuous C-to-T mutation signal on the dU-comprising model sequence, indicating that the strategy of introducing C-to-T mutation signal through chemical reaction in the process of the present invention could indeed achieve the labeling at dU position.

In summary, by capturing this highly characteristic C-to-T mutation signal, very sensitive and accurate dU detection could be achieved.

### 2. Specific Detection Signal Generated at CBE Edited Site

In human HEK293T and MCF7 cell lines, several representative sgRNAs were selected for testing the detection of off-target effect of high-performance CBE tool BE4max by the method provided by the present invention. The method of transfecting cells with the CBE4max editing system was referred to (Xiao Wang, et al. Nature biotechnology 36, 946-949, doi: 10.1038/nbt.4198 (2018)). The representative sgRNAs were “VEGFA_site_2” (SEQ ID NO:23) and “HEK293 site_4” (SEQ ID NO:24) known to have very low specificity in vivo, “EMX1” (SEQ ID NO:25) with medium specificity, “RNF2” (SEQ ID NO:26) which had not been reported to have off-target sites, and “RUNX1” (SEQ ID NO:27) which had been less studied before.

The detection results were shown in FIG. 4, and it could be seen from FIG. 4a that the method of the present invention had caused a very obvious reads enrichment peak (peak) at the corresponding on-target editing site, and after further amplification, obvious and characteristic continuous C-to-T mutation signals could be observed; in addition, these enrichment mutation signals were not observed in the NT samples (i.e. samples transfected with BE4max and non-target sgRNA) as negative controls, indicating that the present invention had very good detection specificity. By comparing with the on-target editing results of these sgRNAs in previous studies, we found that the C with the strongest C-to-T mutation signal was usually the cytosine position with the highest real editing efficiency. And it could be because the polymerase nick translation reaction in the present invention could incorporate a plurality of d5fCTPs at one time, even if only one or two Cs were edited, an obvious continuous C-to-T mutation signals would be generated. It could be seen from FIG. 4b that generally 2-6 continuous C-to-T mutations were mainly generated in the 4-9 bp region behind the edited C.

In addition, taking FIG. 4c as an example, it could be clearly seen that the continuous C-to-T mutation characteristic signals generated by the present invention could be easily distinguished from SNV. And from the perspective of the whole genome level, the signal generated by the method of the present invention under the same amount of data was much stronger than that of the conventional WGS sequencing, could be more easily distinguished from the sequencing background error, and required lower sequencing coverage (FIG. 4d).

In summary, the above observations showed that the signal characteristics generated by the method of the present invention could greatly enhance the detection signal at the editing site, thereby greatly improving the detection sensitivity and reducing the detection cost of the present invention.

### 3. Evaluation of Cas-Dependent and Cas-Independent Off-Targets Caused by CBE

The properties of the off-target sites detected by the present invention at the genome-wide level and their possible production mechanisms can be verified by performing comparison experiments on the deletion of different components of the CBE system. Specifically, we removed the APOBEC1, UGI, and sgRNA parts in the BE4max system, respectively, when transfecting the cells, the constitution of the plasmids after the removal were shown in FIG. 5. In the mean time, Vector samples transfected with only mCherry plasmid were used as negative control samples, and then the genomic DNA of the cells transfected with these samples was respectively detected by using the method of the present invention.

The detection results of Cas-independent off-targets were shown in FIG. 6, which presented three obvious characteristics: 1) The gene position where the signal was located had almost no similarity with the sgRNA sequence (FIG. 6a); 2) Usually, the signal intensity was very low, and most were just above the background level (FIG. 6a); 3) They tended to appear in transcriptionally active regions (FIG. 6e). These features were consistent with previously reported Cas-independent off-target manifestations. More importantly, when this type of off-target sites was further analyzed, it could be seen that when all the components of the CBE system were present, the number of such off-target sites found was more, and it showed a very obvious “TC” motif; when the sgRNA component was removed, the number of such sites was still large, and the motif still existed; but after the APOBEC1 component was deleted, the number of such sites was reduced to the background, and the motif also disappeared (FIGS. 6b to 6d). It was known that APOBEC1 had a natural substrate binding preference for the “TC” motif. These experimental data and characteristics indicated that such off-target sites did not depend on the Cas system, but only on APOBEC1, which should be off-target editing randomly generated by the overexpression of APOBEC1.

The detection results of Cas-dependent off-target sites were shown in FIG. 7, which exhibited the following characteristics: 1) Most of them had a signal intensity much stronger than that of Cas-independent off-target sites. At some sites, signal intensity comparable to that of on-target sites could even be observed (FIG. 7a), indicating that the editing efficiency of such off-target sites would be much higher; 2) Signals were stably and repeatedly generated in the biological replication groups (FIG. 7b); 3) Gene sequences with a certain similarity to sgRNA could usually be found in the genomic region where the signal was located. Through the comparison experiments of component deletions, it could be seen that compared with the samples with all components, the signal intensities of such sites in (−)sgRNA samples and (−)APOBEC samples were all reduced to below the background level, and the signal intensities of (−)UGI samples decreased to different degrees; whereas, the signal intensities of dU modification sites endogenously present in the cells were almost completely unaffected by the component deletions (FIG. 7c). These experimental data indicated that the generation of such type of off-target sites should depend on both sgRNA and APOBEC, and they should indeed belong to classic Cas-dependent off-target. In addition, for sgRNAs with different specificities, the number of Cas-dependent off-target sites identified by the present invention would also change accordingly: for example, under the same bioinformatics analysis identification rule (cutoff), for “VEGFA_site_2”, which was known to have very poor specificity, the present invention identified a total of 511 such off-target sites (FIG. 7b); while for “RNF2”, which was known to have excellent specificity, the present invention did not detect such off-target sites.

### 4. Verification Results of Off-Target Sites

In order to verify the authenticity of the detection results of the method of the present invention, targeted deep sequencing technology was used to measure the actual editing efficiency at the off-target sites identified by the present invention. The so-called targeted deep sequencing technology was to perform targeted PCR amplification on the target site to be tested, and then perform high-throughput sequencing on the PCR product thereof, so that the sequencing depth of at least tens of thousands of reads could be covered at the genomic site to be tested, so that very precise editing efficiency at this site could be obtained.

The results of using the targeted deep sequencing to verify the sites detected by the method of the present invention were shown in FIG. 8. It could be seen from the figure that among the randomly selected sites (151 in total) of the present invention with signal intensities from low to high, 50/50 “EMX1” sites, 51/51 “VEGFA_site_2” sites, 43/43 “HEK293 site_4” and 7/7 “RUNX1” sites were successfully verified by the targeted deep sequencing method, with nearly 100% true positive rate. Moreover, when the actual editing efficiency was still at a low level, the corresponding signal intensity of the present invention was already very high, which further demonstrated that the present invention did have very high detection sensitivity.

In addition, it was verified by the targeted deep sequencing that the generation of the Cas-dependent off-target sites (more than 20 sites were selected) identified by the method of the present invention was indeed dependent on sgRNA. FIG. 9 showed the deep sequencing signals at two sites for the samples with or without sgRNA, and the results of FIG. 9 showed that the generation of the two off-target sites were indeed dependent on sgRNA. In summary, the above data proved the high reliability of the method of the present invention.

FIG. 10 showed the distributions of “EMX1”, “VEGFA_site_2” and “HEK293 site_4” sgRNA on-target editing sites and Cas-dependent off-target editing sites detected at the genome-wide level by the method of the present invention on each chromosome.

### 5. Comparison of Detection Results Between the Method of the Present Invention (Detect-seq) and Other Related Methods

GUIDE-seq is an off-target detection technology widely known in the field of gene editing. and it is mainly used to detect Cas-dependent off-target caused by the CRISPR/Cas9 nuclease system. Since the CBE tool is also constructed based on the inactivated or partially inactivated Cas9 protein, some scholars directly evaluate the off-target effect of the CBE system through the sites identified by GUIDE-seq. But in fact, even if the same sgRNA is used, the genome-wide off-target caused by the CBE system and the off-target caused by the Cas9 nuclease are still very different (Kim, D. et al. Nature biotechnology 35, 475-480, doi: 10.1038/ nbt.3852 (2017)).

The comparison between the method of the present invention and the detection results of GUIDE-seq was shown in FIG. 11a. For “VEGFA_site_2” and “EMX1”, the method of the present invention detected most of the Cas-dependent off-target sites in the GUIDE-seq results; for “HEK293 site_4”, the method of the present invention detected about half of the sites of GUIDE-seq: and the method of the present invention newly discovered a lot of off-target sites that had not been reported by GUIDE-seq. The results of targeted deep sequencing verification on randomly picked sites showed that compared with GUIDE-seq, the 41 new off-target sites detected by the method of the present invention were indeed real off-target sites, while for 15/17 sites that were not reported by the method of the present invention but reported by GUIDE-seq, CBE editing events indeed did not occur in the living cells; and 37 off-target sites identified by both were all successfully verified.

The comparison of detection results between the method of the present invention and the Digenome-seq developed by Kim et al. for the CBE system was shown in FIG. 11b. Digenome-seq was essentially an in vitro off-target detection technology based on WGS. Similar to the comparison results with conventional WGS, the signal values of the present invention at off-target sites were much higher than those of Digenome-seq under the same sequencing amount. The method of the present invention detected most of the Cas-dependent off-target sites reported by Digenome-seq, and newly discovered off-target sites far more than the latter (FIG. 11b). The results of targeted deep-sequencing verification on randomly selected sites showed that: for 10/15 sites that were not reported by the present invention but reported by Digenome-seq. CBE editing events indeed did not occur in the living cells; and 18 off-target sites identified by both were all verified successfully.

On the other hand, the above results also showed that the true positive rate of the report of the present invention was close to 100%, while the true negative rate was about 80%. It was worth mentioning that if the detection results of the method of the present invention were further carefully checked, detection signals of different degrees could actually also be observed at the 7 real off-target sites that had not been successfully reported, but they were not reported possibly due to the failure to reach the cutoff of the bioinformatic analysis.

### 6. Evaluation of Off-Target Effect of the Optimized Version of CBE Tool

Recently, many improved CBE tools have been reported in the field that are excellent in reducing DNA or RNA off-target effects. Among them, YE1-BE4max has been reported as the most comprehensive optimal CBE version by many independent studies (Doman, et al. Nature biotechnology 38, 620-628, doi:10.1038/s41587-020-0414-6 (2020); Zuo. E. et al. Nat Methods 17. 600-604, doi:10.1038/s41592-020-0832-x (2020)).

It could be detected by the method of the present invention that YE1-BE4max did reduce most of the off-target signal levels caused by WT-BE4max. However, taking “EMX1” sgRNA as an example, among the 48 Cas-dependent off-target sites identified from WT-BE4max samples, there were still 4, 3, and a dozen sites that retained detection signals with high, medium, and low intensities in YE1-BE4max (FIG. 12a).

The verification results of targeted deep sequencing showed that: under the condition that the editing efficiency of the on-target sites was similar, YE1-BE4max indeed did not produce editing results at the negative sites (e.g., the “EMX1 pRBS_1” site) reported by the method of the present invention; whereas, at the 3 strong signal sites identified by the present invention (“EMX1 pRBS_4”, “EMX1 pRBS_3” and “EMX1 pRBS_2” sites), YE1-BE4max still showed a very high off-target editing ratio (up to nearly half of the on-target editing efficiency), and one site (EMX1 pRBS_2″ site) among them even showed no decrease at all compared to WT-BE4max. It could be seen that the overall off-target effect of the newly optimized tool evaluated by the present invention had a higher reliability. And similarly, the comprehensive off-target assessment for other optimized versions of CBE tools (e.g., the CBE system constructed using APOBEC3A) could also be performed through the present invention.

In addition, these data also showed that it was not comprehensive enough to evaluate the off-target effects of CBE tools by randomly selecting some sites identified by GUIDE-seq, and the conclusions obtained could be different depending on the selected sites. However, the present invention could provide an evaluation platform based on comprehensive consideration at the whole genome level, and provide a consideration basis for the optimization and comparison of CBE tools.

### 7. Off-Target Detection of CBE Tools Constructed Based on Other CRISPR Systems

In view of the same APOBEC deamination editing principle, CBE tools based on other CRISPR systems, such as Cpf1(Cas12a)-BE, can also use the method of the present invention for off-target assessment. FIG. 13 showed that 949 and 240) Cas-dependent off-targets caused by LbCpf1-BE at the genome-wide level for “RUNX1” (SEQ ID NO: 37) and “DYRK1A” (SEQ ID NO: 38) crRNAs were detected by the method of the present invention. Similarly, targeted deep sequencing verified that 18/18 of them were true off-target editing sites.

### 8. Off-Target Detection of CRISPR-Free DdCBE Tool

HEK293T cells were transfected with the DdCBE system targeting different mitochondrial DNA sites. The transfection method was referred to (Mok, B. Y. et al. Nature 583, 631−+, doi:10.1038/s41586-020-2477-4 (2020)). Three days later, the genome was extracted to detect the editing efficiency at the mitochondrial targeting sites. Sanger sequencing results showed that the editing efficiency was between 35% and 55%. Since the deaminase DddA in the DdCBE system would convert dC on the double-stranded DNA into dU, the method of the present invention could also be used to detect the intermediate product dU, and then evaluate the off-target caused by DdCBE.

Although DdCBE was a mitochondrial DNA cytosine editing tool, the results of Detect-seq revealed that each DdCBE had hundreds of off-target edits in the nucleus. According to the characteristics and causes of off-target signals, off-target signals could be divided into two categories, namely TALE-array sequence (TAS) dependent off-target and TALE-array sequence (TAS) independent off-target. In the present invention, 36 off-target sites were randomly selected for verification, and the results of targeted deep sequencing confirmed that these 36 sites did have a certain proportion of off-target editing, and the off-target efficiency of some sites was even as high as 8%, indicating that Detect-seq could indeed be used to detect the off-target caused by DdCBE. FIG. 14 exemplarily showed the sequencing signal diagrams of TALE-array sequence (TAS) dependent off-target and TALE-array sequence (TAS) independent off-target detected by the method of the present invention and the sequencing results verified by targeted deep sequencing.

### Example 2: Detection of ABE Edited Site

### Experimental Method:

### 1. DNA Fragmentation

Genomic DNA was extracted from living cells of HEK293T (purchased from ATCC, Catalog No.: CRL-11268) transfected with the ABE system. The method of transfecting cells with the ABE system was referred to (Xiao Wang, et al. Nature biotechnology 36, 946-949, doi: 10.1038/nbt.4198 (2018)), and the method of extracting genomic DNA from the cells was referred to the kit manual (purchased from JiangSu CoWin Biotech (CWBIO), Catalog No.: CW2298M).

The extracted genomic DNA was fragmented into fragments with a length of about 300 bp by a Covaris ME220 ultrasonic breaker, and then recovered by DNA Clean & Concentrator-5 Kit.

### 2. End Repair of DNA Fragment

In this step, NEB end repair module and E.coli DNA ligase were used to fill-in some nicks and overhangs of the fragmented DNA, and to repair the genomic DNA damage possibly caused by the fragmentation process.

The reaction system was prepared according to Table 9:

TABLE 9
End repair reaction system

| Component | Total system (100 μL) |
|---|---|
| DNA fragmented in step 1 | 78 μL (~5 ug) |
| Spike-in model sequence (SEQ ID NO: 1 | 3 μL (10 pg of each |
| and one or more of SEQ ID NOs: 28-30) | model sequence) |
| End Repair Reaction Buffer | 10 μL |
| End Repair Enzyme Mix | 5 μL |
| 50 mM NAD⁺ | 2 μL |
| E. coli DNA ligase | 2 μL |

The above reaction system was mixed on ice, reacted at 20° C. for 30 min, and then recovered with 2.0×AMPure XP beads and eluted with 40 μL ddH₂O.

### 3. Addition of dA Tail

The DNA fragment obtained in step 2 was added with a dA at 3′ end, so as to facilitate the subsequent ligation of sequencing adapter (Adaptor) using the A/T complementarity rule. The experimental procedure was the same as in Example 1.

### 4. DNA Damage Repair

The reaction system was prepared according to Table 10:

TABLE 10
Damage repair reaction system

| Component | Total system (50 μL) |
|---|---|
| DNA prepared in Step 3 | 40 μL (~3.3 μg) |
| NEBuffer 3.0 | 5 μL |
| 50 mM NAD⁺ | 1 μL |
| 2.5 mM dNTPs | 1 μL |
| Bst full-length polymerase | 1 μL |
| Taq DNA ligase | 2 μL |

The above reaction system was mixed and reacted at 37° C. for 60 min, then at 45° C. for 60 min, recovered with 2.0×AMPure XP beads, and eluted with 17 μL of ddH₂O, and 1 μL of the sample was taken as input for subsequent library construction.

### 5. Identification of dI

The purpose of this step was to break the second phosphodiester bond at the 3′ end of dI, thereby generating a nick for subsequent labeling.

The reaction system was prepared according to Table 11:

TABLE 11
Nick formation reaction system

| Component | Total system (20 μL) |
|---|---|
| DNA prepared in Step 4 | 16 μL (~3 μg) |
| NEBuffer 4 | 2 μL |
| Endonuclease V (purchased from | 2 μL |
| NEB, Catalog No.: M0305) | |

The above reaction system was mixed, then reacted at 37° C. for 80 min, purified with twice volume of XP beads, and finally eluted with 43 μL of water.

### 6. Biotin-Labeling

The purpose of this step was to incorporate a biotin-labeled dUTP at the position to be detected.

The reaction system was prepared according to Table 12:

TABLE 12
Biotin-labeling reaction system

| Component | Total system (50 μL) | |
|---|---|---|
| DNA prepared in Step 5 | 42 μL (~2.7 μg) | |
| NEBuffer 3 | 5 | μL |
| 100 mM dATP | 0.5 | μL |
| 100 mM dCTP | 0.5 | μL |
| 100 mM dGTP | 0.5 | μL |
| 5 μM Biotin-16-AA-2′-dUTP | 0.5 | μL |
| Full length Bst DNA polymerase | 1 | μL |

The above reaction system was mixed, and then reacted at 37° C. for 40 min; after the end of the reaction, 1 μL of 50 mM NAD⁺ and 2 μL of Taq DNA ligase were added to the tube, continuously incubated in the PCR instrument at 37° C. for 40 min, then purified with 2×XP beads, and finally eluted with 41 μL of water.

### 7. Enrichment of Fragment

Each PD (pull down) sample corresponded to 10 μL of Streptavidin C1 beads. A sufficient amount of beads were taken and washed three times with 1×B&W buffer (5 mM Tris-HCl (pH 7.5), 1 M NaCl, 0.5 mM EDTA, 0.05% Tween-20), then resuspended with 40 μL 2×B&W buffer, then added with an equal volume of sample DNA treated in step 6 above, mixed well, and incubated at room temperature for 1 h with rotation. The magnetic beads were then washed three times with 1×B&W buffer, and then once with 10 mM Tris-HCl (pH 8.0), and rotated at room temperature for 5 min each time. Finally, the Tris-HCl liquid was sucked out on a magnetic stand, and the remaining magnetic beads bound with DNA fragments were used for adapter ligation reaction.

### 8. Ligation of Adapter

1) The adapter stock solution (30 μM) was diluted to 1.5 μM with 10 mM Tris-HCl on ice. The Y-type adapter used was obtained by annealing two single-strand sequences, wherein the 5′ end of the forward single-strand had a phosphorylation modification, its sequence was shown in SEQ ID NO: 7, and the reverse single-strand sequence was shown in SEQ ID NO:8.

2) NEBNext® Quick Ligation Module was used to perform adapter ligation reaction on the Input sample (aqueous solution) retained in step 4 and the PD sample (connected to magnetic beads) obtained in step 7 above.

The reaction system was prepared according to Table 13:

TABLE 13
Adapter ligation reaction system

| Component | Total system (25 μL) | |
|---|---|---|
| ddH₂O | 14 | μL |
| NEB Quick Ligation Buffer | 5 | μL |
| 1.5 μM Y-type adaptor | 2.5 | μL |
| Quick T4 DNA Ligase | 2.5 | μL |
| PD or Input sample DNA | 1 | μL |

For the adapter ligation reaction of the PD sample: the above reaction system was mixed and reacted at about 20° C. for 1 h with rotation (to avoid magnetic beads sedimentation), then supplemented with 50 μl of 1×B&W buffer, continuously incubated at room temperature for 1 h with rotation (to allow a small amount of DNA fragments that were separated during the ligation process to connect with the magnetic beads again), and then underwent the next reaction;

For the adapter ligation reaction of the Input sample: the above reaction system was mixed, then placed in a PCR machine and reacted at 20° C. for 1 h, and subjected to recovery and retention with 1×AMPure XP beads so as to remove the adapters that had not been successfully ligated.

### 9. Washing and Purification Process

The sample connected to the beads after the above step 8 (PD sample) was washed three times with 1 mL of 1×BW, then washed once with 200 μL of EB (10 mM Tris-HCl), and finally the DNA library in the PD sample was eluted in the shaker under conditions of using 25 μL of ddH20 at 95° C. at 1200 rpm.

### 10. Library Amplification

The experimental procedure was the same as in Example 1.

### 11. Quality Check of Library

Qubit2.0 precision spectrophotometer was used to measure library concentration;

Fragment Analyzer 12 automatic capillary electrophoresis instrument was used to check the distribution of library fragments;

qPCR was used to perform relative quantification of the model sequence and calculate the enrichment fold-change. The primers used in qPCR were shown in SEQ ID NOs:11-12, 31-36. The data processing adopted the 2^{-ΔΔCt} method. The enrichment fold-change was a fold-change of the relative amount of the spike-in DNA molecule that comprised a specific type of modification in the PD sample (using the Control model sequence as a reference) compared to the corresponding Input sample, and based on this fold-change, the enrichment of this batch of experiments could be evaluated;

The model sequence underwent full-length PCR amplification, the obtained PCR product underwent Sanger sequencing, and the labeling status of this batch of experiments could be evaluated through the sequencing results;

Finally, the resulting library was delivered to the Illumina Hiseq X-ten platform for paired-end sequencing (read length 150 bp).

### Processing and Analysis of Sequencing Data:

### 1. Back-Pasting and Filtering of Data in the Present Invention

After the data was downloaded from machine, the cutadapt (version 1.18) software was firstly used to remove the sequencing adapters from the sequencing reads (reads) in the FASTQ file of the sequencing results. The specific command parameters were: cutadapt—times 1 −e 0.1 −O 3—quality-cutoff 25 −m 50. After removing the adapters, the sequencing reads were back-pasted to the reference genome (version number was hg38) using BWA MEM (version 0.7.17), and the alignment results with an alignment quality MAPQ greater than 20, that was, an alignment error rate less than 1%, were retained for downstream analysis. Next, the screened high-quality alignment results underwent a deduplication processing that was carried out with the Picard MarkDuplicates command (version 1.9). The main purpose of this step was to remove the molecular redundancy caused by amplification during the library construction process. After the above steps, the genome back-pasting results (BAM format files) were obtained for downstream analysis.

### 2. Preliminary Identification of Signals of the Present Invention

After obtaining the back-pasted and filtered BAM files, the samtools mpileup −q 20 −Q 20 command (version 1.9) was first used to convert the BAM files to mpileup files. Then, the parse-mpileup command and the bmat2pmat command in the aforementioned software tool were used to generate pmat files. Then the pmat-merge command of the software tool was used to scan and organize all the concatenated C to T mutation signals in the whole genome and record them into mpmat format files. Finally, the mpmat-select command of the software tool was used for screening, thereby obtaining the preliminary sequencing signals of the present invention.

### 3. Identification of Enrichment Signals of the Present Invention

After obtaining the preliminary sequencing signals of the present invention, it was necessary to perform enrichment detection on these candidate regions. First, the find-significant-mpmat command in the software tool was used to perform a statistical test on the candidate regions, and the results of the statistical test were corrected by the BH method to obtain a false discovery rate (FDR). Finally, it was considered that the regions with the FDR less than 0.01, the enrichment fold-change of the treatment group greater than 2 after normalization compared with the control group, the reads with mutation signal in the sample of the control group less than 3, and the sequencing reads with mutation signal in the treatment group not less than 5, were the final identification regions of the present invention.

4. Alignment of Off-Target Site Gene Sequence and sgRNA Sequence

In the enrichment signal region identified in the above steps, the binding site of sgRNA could be deduced by sequence alignment. This deduced sgRNA binding site was called pRBS (putative sgRNA binding site). When performing sequence alignment between sgRNA and the enrichment signal region, an improved semi-global alignment method was used. Firstly, the PAM sequence (NAG/NGG) was searched in the enrichment region, and then for the found PAM site, the sequence of 30 nt in the 5′ direction of the PAM was extracted to perform semi-global double-sequence alignment with the sgRNA, and the optimal result reported in the alignment was pRBS; if no PAM was found in the region, the sgRNA was directly used to perform the semi-global alignment with the sequence of the region, and the optimal result of the alignment was the pRBS of sgRNA. The alignment parameters used for this step were: match +5; mismatch −4; open gap −24; gap extension −8. The alignment program for this step was comprised in the mpmat-to-art command in the Detect-seq software toolbox.

### Experimental Results:

1. Specific Labeling and Enrichment of dI-Comprising Model Sequence

In order to prove the specificity and efficiency of the method of the present invention, model sequences and control sequences (SEQ ID NOs: 1. 28-30) comprising different modified bases were incorporated into the library construction samples. Finally, the proportion changes of different model sequences in samples before and after pull-down were calculated and compared by qPCR technology (relative quantification was performed with the control sequence without any modification (Control model sequence as shown in SEQ ID NO: 1)), and the enrichment fold-changes of different model sequences in the samples before and after pull-down were calculated. The enrichment fold-changes were shown in FIG. 16. It could be seen from the figure that for the model sequences comprising single dI:dC and dI:dT base pairs, the method of the present invention could enrich them by about 220 times and about 50 times or more, respectively, whereas, the model sequences comprising only Nick were almost not enriched at all, so that it was proved that the method of the present invention could specifically and efficiently enrich dI-comprising DNA fragments.

### 2. Enrichment of DNA Comprising Sites Actually Edited by ABE

Genomic DNA of HEK293T cells transfected by ABEmax was extracted. The method of transfecting cells with ABEmax were referred to (Xiao Wang, et al. Nature biotechnology 36, 946-949, doi: 10.1038/nbt.4198 (2018)). By using the second-generation sequencing library constructed by the method of the present invention, and after a series of bioinformatics analysis, the information of sites edited by ABEmax at the genome-wide level could be obtained. FIG. 17 showed the high-throughput sequencing results of ABE at the on-target of HEK293_site_4 (referred to as HEK4) (SEQ ID NO:24). It could be seen from the figure that no mutation signal was detected in the negative control vector sample, and there was an A-to-G mutation signal in all-PD sample of the experimental group, wherein the mutation site is the editing site; in addition, compared with the vector sample, the number of reads comprising mutation in the all-PD sample was significantly increased, which also showed that the enrichment did occur.

FIG. 18 showed the high-throughput sequencing results of one of the off-target sites. It could be seen from the figure that there was no mutation signal in the vector sample, while the all-PD sample contained A-to-G mutation information, which was the off-target signal.

### 3. Verification Results of Off-Target Sites Detected by the Method of the Present Invention

FIG. 19 showed the verification results of one of the off-target sites detected by the method of the present invention by targeted deep sequencing. It could be seen from the figure that the off-target editing rate of this site was as high as 10.82%, and from the comparison of the on-target sequence in the figure and the off-target sequence here, it could be seen that the two were very close, and thus it was speculated that the off-target here was a cas-dependent off-target.

### 4. Evaluation of Off-Target Effects of Various ABE Systems

In addition to the ABEmax system, the two new tools ABE8e and ACBE, as well as other base editing systems based on adenine deaminase that may be developed in the future, can use the present invention to identify off-target sites.

FIGS. 20 to 22 showed the high-throughput sequencing results at detected on-target and off-target sites when the method of the present invention was used for the off-target detection of two new tools, ABE8e (Richter et al., 2020) and ACBE (Grunewald et al., 2020; Li et al., 2020; Sakata et al., 2020; Zhang et al., 2020). For the on-target sites, it could be observed from FIG. 20 that these three systems had corresponding A-to-G mutation signals inside the sgRNA binding regions, wherein the signal of ABE8e was stronger than that of ABE, while ACBE also had C-to-T mutation signal besides the A-to-G mutation signal.

For off-target sites, such as the above-mentioned off-target 4 site, off-target signals were also detected in these three systems, just with different signal intensities (FIG. 21). In addition to the off-target sites shared by the three systems, the present invention also detected the unique off-target sites of ABE8e. As shown in FIG. 22, the off-target signal at this site was only detected in the sample transfected with the ABE8e system, while the corresponding off-target signal was not detected in the other two samples. Previous literature reports that the activity of ABE8e is much higher than that of ABE, and the present invention indeed detects much more off-target signals of ABE8e, which confirms the reliability of the present invention to a certain extent.

### Example 3

After the inventors of the present application replaced step 7 (malononitrile reaction) of the experimental method in Example 1 with other 5fC labeling methods, the C to T mutation signal could also be induced at d5fC without affecting the enrichment results, and the labeling at dU site could also be finally achieved.

Taking chemical labeling methods such as pyridine borane labeling as an example, the inventors replaced malononitrile in Example 1 with pyridine borane or 2-picoline borane to perform the reaction (other experimental steps were referred to Example 1). The characterization results of the spike in model sequences processed by the method of the present invention were shown in FIG. 23. FIG. 23 showed that: 1) the model sequences comprising single dU:dA (SEQ ID NO: 2) and dU:dG (SEQ ID NO: 5) base pairs were enriched by about 60 times and 20 times, respectively, while the model sequence (SEQ ID NO: 4) comprising AP was almost not enriched at all (FIG. 23a): 2) according to the results of Sanger sequencing, continuous C-to-T mutation signals were observed on the model sequences comprising dU (FIG. 23b). The above results showed that the present invention could also introduce continuous C-to-T mutation signals without affecting the enrichment results by using other similar chemical reactions, and could finally realize the labeling of dU site. It should be pointed out that, compared with the malononitrile labeling method, the proportion of C-to-T mutation signal generated by the pyridine borane labeling method was lower (FIG. 23b).

### Example 4

The Biotin-dU labeling molecules in Examples 1 and 2 could also be replaced with other labeling molecules with enrichment effects. For example, after the inventors of the present application replaced Biotin-dU in Example 1 with Biotin-dG, the model sequences comprising single dU:dA (SEQ ID NO: 3) and dU:dG (SEQ ID NO: 5) base pairs were also enriched by about 30 times and 20 times, respectively, while the model sequences comprising AP site (SEQ ID NO: 4) and Nick (SEQ ID NO: 30) were almost not enriched at all (FIG. 24). This result showed that after using Biotin-dG, the present invention would also specifically enrich dU-comprising DNA fragments.

Although the specific models for carrying out the present invention have been described in detail, those skilled in the art will understand that: according to all the teachings that have been disclosed, various modifications and changes can be made to the details, and these changes are all within the protection scope of the present invention. The full scope of the present invention is given by the appended claims and any equivalents thereof.

What is claimed is:

1. A method for detecting an editing site, editing efficiency or off-target effect of a base editor editing a target nucleic acid, which comprises the following steps:
(1) providing an edit product of the base editor editing the target nucleic acid, in which the edit product comprises a base editing intermediate, and the base editing intermediate comprises a first nucleic acid strand and a second nucleic acid strand; wherein, the first nucleic acid strand comprises an edited base generated by the base editor editing the target nucleic acid;
(2) generating a single-strand break in a segment comprising the edited base (e.g., in a segment from 10 nt upstream to 10 nt downstream of the edited base) in the first nucleic acid strand;
(3) introducing a nucleotide labeled with a first labeling molecule at or downstream of the single-strand break, to produce a labeled product comprising the first labeling molecule;
(4) isolating or enriching the labeled product; for example, isolating or enriching the labeled product by using a first binding molecule capable of specifically recognizing and binding the first labeling molecule;
(5) determining a sequence of the labeled product;
thereby, determining the editing site, editing efficiency or off-target effect of the base editor editing the target nucleic acid;
preferably, the base editor is a single base editor or a dual base editor.

2. The method according to claim 1, wherein the base editor is a cytosine base editor, an adenine base editor, or an adenine and cytosine dual base editor.

3. The method according to claim 1 or 2, wherein the target nucleic acid is a genomic nucleic acid or a mitochondrial nucleic acid.

4. The method according to any one of claims 1-3, wherein the edit product is a product of the base editor editing the target nucleic acid outside a cell, inside a cell, or inside an organelle (e.g., a nucleus or a mitochondria).

5. The method according to any one of claims 1-4, wherein the method further comprises the following step before step (1): under a condition that allows the base editor to edit the target nucleic acid, the base editor is contacted with the target nucleic acid to generate the edit product;
preferably, under a condition that allows the base editor to edit the target nucleic acid, the base editor is contacted with the target nucleic acid inside a cell, outside a cell or inside an organelle (e.g., a nucleus or a mitochondria) to generate the edit product;
for example, before the step (1), the method further comprises the following steps: the base editor is introduced into a cell or into an organelle, so that the base editor is contacted with the target nucleic acid in the cell or in the organelle to perform base editing, thereby generating the edit product; or, a nucleic acid molecule encoding the base editor is introduced into a cell or into an organelle and made to express the base editor, so that the base editor is contacted with the target nucleic acid in the cell or in the organelle to perform base editing, thereby generating the edit product;
preferably, in step (1), the target nucleic acid after the base editing is extracted or isolated from the cell or organelle, and optionally, fragmented, so as to obtain the edit product;
preferably, in step (1), the target nucleic acid after the base editing is extracted or isolated from the cell or organelle, and undergoes nucleic acid fragmentation and end repair (e.g., filling-in of 5′ end overhang and/or excision of 3′ end overhang) to obtain the edit product;
preferably, the second nucleic acid strand did not undergo base editing or did not comprise an edited base;
preferably, the edited base is selected from the group consisting of uracil or inosine.

6. The method according to any one of claims 1-5, wherein, in step (2), the single chain break is generated at or upstream (e.g., within 10 nt upstream) or downstream (e.g., within 10 nt downstream) of the site of the edited base;
preferably, before performing step (2), the method further comprises: a step of repairing a possible single-strand break (SSB) (e.g., an endogenous single-strand break) in the edit product; for example, before performing step (2), the method further comprises: using a nucleic acid polymerase, a nucleotide (e.g., a nucleotide without label) and a nucleic acid ligase to repair a possible SSB (e.g., endogenous SSB) in the edit product;
preferably, in step (2), using an endonuclease (e.g., endonuclease V, endonuclease VIII or AP endonuclease) to generate the single-strand break in the first nucleic acid strand.

7. The method according to any one of claims 1-6, wherein the nucleotide labeled with the first labeling molecule is selected from the group consisting of uracil deoxyribonucleotide labeled with the first labeling molecule (e.g., dUTP labeled with the first labeling molecule), cytosine deoxyribonucleotide labeled with the first labeling molecule (e.g., dCTP labeled with the first labeling molecule), thymidine deoxyribonucleotide labeled with the first labeling molecule (e.g., dTTP labeled with the first labeling molecule), adenine deoxyribonucleotide labeled with the first labeling molecule (e.g., dATP labeled with the first labeling molecule), guanine deoxyribonucleotide labeled with the first labeling molecule (e.g., dGTP labeled with the first labeling molecule), or any combination thereof;
preferably, the nucleotide labeled with the first labeling molecule is uracil deoxyribonucleotide labeled with the first labeling molecule (e.g., dUTP labeled with the first labeling molecule) or guanine deoxyribonucleotide labeled with the first labeling molecule (e.g., dGTP labeled with the first labeling molecule);
preferably, the first labeling molecule and the first binding molecule constitute a molecular pair capable of specific interaction (e.g., capable of specifically binding to each other); for example, the first labeling molecule is biotin or functional variant thereof, and the first binding molecule is avidin or functional variant thereof; or, the first labeling molecule is a hapten or antigen, and the first binding molecule is an antibody specific for the hapten or antigen; or, the first labeling molecule is an alkynyl-comprising group (e.g., ethynyl), and the first binding molecule is an azido-comprising compound capable of undergoing a click chemical reaction with the alkynyl; for example, the nucleotide labeled with the first labeling molecule is an ethynyl-comprising nucleotide (e.g., 5-Ethynyl-dUTP), and the first binding molecule is an azide compound (e.g., azido-modified magnetic beads) capable of undergoing a click chemical reaction with the ethynyl;
preferably, the nucleotide labeled with the first labeling molecule is introduced at or downstream of the single-strand break through a nucleic acid polymerization reaction, thereby producing a labeled product comprising the first labeling molecule; for example, in step (3), using a nucleic acid polymerase (e.g., a nucleic acid polymerase having strand-displacement activity) to introduce the nucleotide labeled with the first labeling molecule at or downstream of the single-strand break;
preferably, in step (3), a nucleotide labeled with a second labeling molecule is also introduced at or downstream of the single-strand break, thereby generating a labeled product comprising the first labeling molecule and the second labeling molecule;
preferably, the nucleotide labeled with the second labeling molecule is a nucleotide molecule capable of complementary base pairing with a different nucleotide under different conditions (e.g., before and after undergoing a treatment); for example, the nucleotide labeled with the second labeling molecule is selected from the group consisting of 5-formyl cytosine deoxyribonucleotide, 5-carboxycytosine deoxyribonucleotide, 5-hydroxymethylcytosine deoxyribonucleotide, and N4-acetylcytosine deoxyribonucleotide; for example, the nucleotide labeled with the second labeling molecule is 5-formylcytosine deoxyribonucleotide;
preferably, the nucleotide labeled with the second labeling molecule is introduced at or downstream of the single-strand break by nucleic acid polymerization.

8. The method according to any one of claims 1-7, wherein, in step (2), the single-strand break is generated at the site of the edited base; and, in step (3), the nucleotide labeled with the first labeling molecule and the nucleotide labeled with the second labeling molecule are introduced at and downstream of the single-strand break, thereby generating a labeled product comprising the first labeling molecule and the second labeling molecule;
preferably, after step (3), the labeled product is undergoes a treatment to change the complementary base pairing capability of the nucleotide labeled with the second labeling molecule comprised therein;
for example, the nucleotide labeled with the second labeling molecule is 5-formylcytosine deoxyribonucleotide, and, after step (3), the labeled product is treated with a compound (e.g., malononitrile, borane compound (e.g., pyridine borane compound, such as pyridine borane or 2-picoline borane), or azido-indandione) to change the complementary base pairing capability of the 5-formylcytosine deoxyribonucleotide comprised therein;
for example, the nucleotides labeled with the second labeling molecule is 5-carboxycytosine deoxyribonucleotide, and, after step (3), the labeled product is treated with a compound (e.g., borane compound (e.g., pyridine borane compound, such as pyridine borane or 2-picoline borane)) to change the complementary base pairing capability of the 5-carboxycytosine deoxyribonucleotide comprised therein;
for example, the nucleotide labeled with the second labeling molecule is 5-hydroxymethylcytosine deoxyribonucleotide, and, after step (3), the labeled product is first treated with an oxidant (e.g., potassium ruthenate) or oxidase (e.g., TET (ten-eleven translocation) protein), and then with a compound (e.g., malononitrile, borane compound (e.g., pyridine borane compound, such as pyridine borane or 2-picoline boron alkane), or azazido-indandione) to change the complementary base pairing capability of the 5-hydroxymethylcytosine deoxyribonucleotide comprised therein;
for example, the nucleotide labeled with the second labeling molecule is N4-acetylcytosine deoxyribonucleotide (dac⁴C), and, after step (3), the labeled product is treated with a compound (e.g., sodium cyanoborohydride) to change the complementary base pairing capability of the N4-acetylcytosine deoxyribonucleotide comprised therein;
preferably, the step of treating the labeled product is performed before sequencing the labeled product, for example, before step (4) or before step (5);
preferably, before step (3) (e.g., before step (2)), the nucleotide labeled with the second labeling molecule that is possibly presented in the edit product is protected (e.g., endogenous 5-formylcytosine deoxyribonucleotide is protected with ethylhydroxylamine, or, endogenous 5-hydroxymethylcytosine deoxyribonucleotide is protected with βGT-catalyzed glycosylation).

9. The method according to any one of claims 1-7, wherein, in step (2), the single-strand break is generated downstream of the edited base; and, in step (3), the nucleotide labeled with the first labeling molecule is introduced at or downstream of the single-strand break, and optionally, a nucleotide labeled with a second labeling molecule in introduced, thereby generating a labeled product comprising the first labeling molecule and optionally the second labeling molecule.

10. The method according to any one of claims 1-9, wherein, in step (4), the labeled product is isolated or enriched using a first binding molecule attached to a solid support;
for example, the solid support is selected from the group consisting of magnetic beads, sepharose beads, or chip.

11. The method according to any one of claims 1-10, wherein, before performing step (5), the method further comprises: amplifying the labeled product that is isolated or enriched in step (4); and/or, constructing a sequencing library of the labeled product that is isolated or enriched in step (4).

12. The method according to any one of claims 1-11, wherein, in step (5), the sequence of the labeled product is determined by a sequencing method (e.g., second-generation sequencing or third-generation sequencing), a hybridization method or a mass spectrometry method;
preferably, the method further comprises: comparing the sequence determined in step (5) with a reference sequence, so as to determine the editing site, editing efficiency or off-target effect of the base editor editing the target nucleic acid;
preferably, the reference sequence is the target nucleic acid sequence before the base editing; for example, the target nucleic acid sequence before the base editing is be obtained from a database, or is obtained by a sequencing method.

13. The method according to any one of claims 1-12, wherein the base editor is a cytosine base editor (e.g., a nuclear cytosine base editor, an organelle cytosine base editor);
preferably, the cytosine base editor is a cytosine base editor capable of editing cytosine into uracil; preferably, the base editor is a cytosine base editor capable of editing a nuclear nucleic acid or a cytosine base editor capable of editing a mitochondrial nucleic acid;
preferably, the edited base is uracil;
preferably, the base editing intermediate is a nucleic acid molecule (e.g., a DNA molecule) comprising uracil;
preferably, the nucleotide labeled with the second labeling molecule is a modified cytosine deoxyribonucleotide capable of complementary base pairing with a first nucleotide (e.g. guanine deoxyribonucleotide) before undergoing a treatment, and capable of complementary base pairing with a second nucleotide (e.g., adenine deoxyribonucleotide) after undergoing a treatment;
preferably, the nucleotide labeled with the second labeling molecule is selected from the group consisting of d5fC, d5caC, d5hmC and dac⁴C:
preferably, the nucleotide labeled with the second labeling molecule is d5fC.

14. The method according to claim 13, wherein, in step (2), the single-strand break is generated at the site of the edited base in the first nucleic acid strand using an AP site-specific endonuclease (e.g., AP endonuclease); and, in step (3), the nucleotide labeled with the first labeling molecule and the nucleotide labeled with the second labeling molecule are introduced at and downstream of the the single-strand break, thereby generating the labeled product comprising the first labeling molecule and the second labeling molecule;
preferably, before performing step (2), the method further comprises a step of forming an AP site at the position of the edited base in the first nucleic acid strand; for example, before performing step (2), the method further comprises: a step of incubating the edit product with UDG (uracil-DNA glycosylase);
preferably, before the step of incubating with UDG, the method further comprises a step of repairing an AP site that is possibly presented in the edit product; for example, the step of repairing AP site comprises:
(a) incubating an AP endonuclease with the edit product in which an AP is possibly presented under a condition that allows the AP endonuclease to exert its cleavage activity;
(b) incubating the product of step (a) with a nucleic acid polymerase (e.g., DNA polymerase) and a nucleotide molecule (e.g., a nucleotide molecule that is not labeled with the first labeling molecule or the second labeling molecule; for example, dNTP without label) under a condition that allows nucleic acid polymerization;
(c) incubating the product of step (b) with a nucleic acid ligase under a condition that allows the nucleic acid ligase to exert its ligation activity,
thereby, repairing the AP site that is possibly presented in the edit product;
preferably, after step (3), the labeled product is treated to change the complementary base pairing capability of the nucleotide labeled with the second labeling molecule comprised therein;
for example, the nucleotide labeled with the second labeling molecule is 5-formylcytosine deoxyribonucleotide, and, after step (3), the labeled product is treated with a compound (e.g., malononitrile, borane compound (e.g., pyridine borane compound, such as pyridine borane or 2-picoline borane), or azido-indandione) to change the complementary base pairing capability of the 5-formylcytosine deoxyribonucleotide comprised therein;
for example, the nucleotides labeled with the second labeling molecule is 5-carboxycytosine deoxyribonucleotide, and, after step (3), the labeled product is treated with a compound (e.g., borane compound (e.g., pyridine borane compound, such as pyridine borane or 2-picoline borane)) to change the complementary base pairing capability of the 5-carboxycytosine deoxyribonucleotide comprised therein;
for example, the nucleotide labeled with the second labeling molecule is 5-hydroxymethylcytosine deoxyribonucleotide, and, after step (3), the labeled product is first treated with an oxidant (e.g., potassium ruthenate) or oxidase (e.g., TET (ten-eleven translocation) protein), and then with a compound (e.g., malononitrile, borane compound (e.g., pyridine borane compound, such as pyridine borane or 2-picoline boron alkane), or azazido-indandione) to change the complementary base pairing capability of the 5-hydroxymethylcytosine deoxyribonucleotide comprised therein;
for example, the nucleotide labeled with the second labeling molecule is N4-acetylcytosine deoxyribonucleotide (dac⁴C), and, after step (3), the labeled product is treated with a compound (e.g., sodium cyanoborohydride) to change the complementary base pairing capability of the N4-acetylcytosine deoxyribonucleotide comprised therein;
preferably, before step (3) (e.g., before step (2)), the nucleotide labeled with the second labeling molecule that is possibly presented in the edit product is protected; for example, before step (3) (e.g., before step (2)), endogenous 5-formylcytosine deoxyribonucleotide is protected with ethylhydroxylamine, or, endogenous 5-hydroxymethylcytosine deoxyribonucleotide is protected with βGT-catalyzed glycosylation.

15. The method according to any one of claims 1-12, wherein the base editor is an adenine base editor;
preferably, the adenine base editor is an adenine base editor capable of editing adenine into inosine;
preferably, the edited base is inosine;
preferably, the base editing intermediate is a nucleic acid molecule (e.g., a DNA molecule) comprising inosine.

16. The method according to claim 15, wherein, in step (2), the single-strand break is generated at or downstream of the edited base in the first nucleic acid strand using an inosine site-specific endonuclease (e.g., endonuclease V, or endonuclease VIII); and, in step (3), the nucleotide labeled with the first labeling molecule, and optionally, the nucleotide labeled with the second labeling molecule, are introduced at or downstream of the single-strand break, thereby generating the labeled product comprising the first labeling molecule and optionally the second labeling molecule.

17. The method according to any one of claims 1-12, wherein the base editor is a dual base editor;
preferably, the base editor is a base editor capable of editing cytosine into uracil and editing adenine into inosine;
preferably, the edited base is inosine and/or uracil;
preferably, the base editing intermediate is a nucleic acid molecule (e.g., a DNA molecule) comprising inosine and/or uracil;
preferably, the method has the features defined in any one of claims 13-16.

18. A kit, which comprises an enzyme or a combination of enzymes capable of generating a single-strand break in a segment comprising an edited base, a nucleotide labeled with a first labeling molecule and a binding molecule capable of specifically recognizing and binding to the first labeling molecule; wherein, the enzyme or combination of enzymes is capable of specifically recognizing a base editing intermediate comprising the edited base, and capable of generating a phosphodiester bond break in a segment 10 nt upstream to 10 nt downstream of the edited base;
preferably, the nucleotide labeled with the first labeling molecule and the first binding molecule are as defined in claim 7;
preferably, the enzyme or combination of enzymes capable of generating the single-strand break in the segment comprising the edited base is endonuclease V, or endonuclease VIII;
preferably, the enzyme or combination of enzymes capable of generating the single-strand break in the segment comprising the edited base is a combination of UDG enzyme and AP endonuclease;
preferably, the kit further comprises a nucleotide labeled with a second labeling molecule, the nucleotide labeled with the second labeling molecule is a nucleotide molecule (e.g., 5-formylcytosine deoxyribonucleotide) capable of complementary base pairing with different nucleotides under different conditions (e.g., before and after a treatment); preferably, the nucleotide labeled with the second labeling molecule is as defined in claim 7;
preferably, the kit further comprises a nucleic acid polymerase (e.g., a nucleic acid polymerase having strand displacement activity), and/or, a nucleic acid ligase, an unlabeled nucleotide molecule, a reagent for protecting the nucleic acid labeled with the second labeling molecule (e.g., ethylhydroxylamine, reagents (e.g., β-glucosyltransferase, glucosyl compound) required for βGT-catalyzed glycosylation reaction, or any combination thereof), a reagent (e.g., malononitrile, azido-indandione, borane compound (e.g., pyridine borane compound, such as pyridine borane or 2-picoline borane). potassium ruthenate. TET protein, sodium cyanoborohydride, or any combination thereof) for treating the nucleotide labeled with the second labeling molecule so as to change the complementary base pairing capability thereof, or any combination thereof.


SEQUENCE LISTING (38 sequences)

SEQ ID NO: 1 - DNA, 137 bp, Artificial Sequence
  Control model sequence
aactgattgcccgtctccgctcgctgggtgaacaactgaaccgtgatgtcagcatgacgttatctggcggtggagatggctccgtgtggcagagctgaaagaggagcttgatgacacgtaatgcttgcgtggcaaac

SEQ ID NO: 2 - DNA, 137 bp, Artificial Sequence
  dUdA-1 model sequence
  misc_feature (48)..(48): n is uracil deoxyribonucleotide
aactgattgcccgtctccgctcgctgggtgaacaactgaaccgtgatntcagcatgacggcggtaagcacgaactcaggctccgtgtggcagagctgaaagaggagcttgatgacacgggaaataccgtggtgtggc

SEQ ID NO: 3 - DNA, 139 bp, Artificial Sequence
  dUdA-2 model sequence
  misc_feature (48)..(48): n is uracil deoxyribonucleotide
aactgattgcccgtctccgctcgctgggtgaacaactgaaccgtgatntcagcatgacgcatgagtgccctcagcagtagctccgtgtggcagagctgaaagaggagcttgatgacacgtccaacctttaggagccatg

SEQ ID NO: 4 - DNA, 139 bp, Artificial Sequence; identical to SEQ ID NO: 3
  APdA model sequence
  misc_feature (48)..(48): n is AP site

SEQ ID NO: 5 - DNA, 139 bp, Artificial Sequence
  dUdG model sequence
  misc_feature (48)..(48): n is uracil deoxyribonucleotide
aactgattgcccgtctccgctcgctgggtgaacaactgaaccgtgatntcagcatgacggcggctggagcggtaattttgctccgtgtggcagagctgaaagaggagcttgatgacacgtaatgacgttgccagccagt

SEQ ID NO: 6 - DNA, 145 bp, Artificial Sequence
  d5fCdG model sequence
  misc_feature (100)..(100): n is 5-formylcytosine deoxyribonucleotide
catgagtgccctcagcagtaagtaactgaccagatctctcgtgcctcttgaggctactgagttatccaacctttaggagccatgcatcgatagcatccgncacaggcagtgaggctactgagtcatgcacgcagaaagaaatagc

SEQ ID NO: 7 - DNA, 31 bp, Artificial Sequence
  Forward single-strand sequence of Y-type adapter
gatcggaagagcacacgtctgaactccagtc

SEQ ID NO: 8 - DNA, 33 bp, Artificial Sequence
  Reverse single-strand sequence of Y-type adapter
acactctttccctacacgacgctcttccgatct

SEQ ID NO: 9 - DNA, 58 bp, Artificial Sequence
  Universal Primer sequence
aatgatacggcgaccaccgagatctacactctttccctacacgacgctcttccgatct

SEQ ID NO: 10 - DNA, 64 bp, Artificial Sequence
  Index Primer sequence
  misc_feature (25)..(30): n is independently selected from guanine deoxyribonucleotides, adenine deoxyribonucleotides, thymine deoxyribonucleotides, and cytosine deoxyribonucleotides
caagcagaagacggcatacgagatnnnnnngtgactggagttcagacgtgtgctcttccgatct

SEQ ID NO: 11 - DNA, 19 bp, Artificial Sequence
  qPCR Primer-1 for control model sequence
ttatctggcggtggagatg

SEQ ID NO: 12 - DNA, 19 bp, Artificial Sequence
  qPCR Primer-2 for control model sequence
gtttgccacgcaagcatta

SEQ ID NO: 13 - DNA, 19 bp, Artificial Sequence
  qPCR Primer-1 for dUdA-1 model sequence
gcggtaagcacgaactcag

SEQ ID NO: 14 - DNA, 19 bp, Artificial Sequence
  qPCR Primer-2 for dUdA-1 model sequence
gccacaccacggtatttcc

SEQ ID NO: 15 - DNA, 20 bp, Artificial Sequence
  qPCR Primer-1 for dUdA-2 model sequence
catgagtgccctcagcagta

SEQ ID NO: 16 - DNA, 20 bp, Artificial Sequence
  qPCR Primer-2 for dUdA-2 model sequence
catggctcctaaaggttgga

SEQ ID NO: 17 - DNA, 20 bp, Artificial Sequence
  qPCR Primer-1 for APdA model sequence
catgagtgccctcagcagta

SEQ ID NO: 18 - DNA, 20 bp, Artificial Sequence
  qPCR Primer-2 for APdA model sequence
catggctcctaaaggttgga

SEQ ID NO: 19 - DNA, 20 bp, Artificial Sequence
  qPCR Primer-1 for dUdG model sequence
gcggctggagcggtaatttt

SEQ ID NO: 20 - DNA, 20 bp, Artificial Sequence
  qPCR Primer-2 for dUdG model sequence
actggctggcaacgtcatta

SEQ ID NO: 21 - DNA, 20 bp, Artificial Sequence
  qPCR Primer-1 for d5fCdG model sequence
catgagtgccctcagcagta

SEQ ID NO: 22 - DNA, 20 bp, Artificial Sequence
  qPCR Primer-2 for d5fCdG model sequence
catggctcctaaaggttgga

SEQ ID NO: 23 - DNA, 20 bp, Artificial Sequence
  VEGFA_site_2 sgRNA target site sequence
gaccccctccaccccgcctc

SEQ ID NO: 24 - DNA, 20 bp, Artificial Sequence
  HEK293 site_4 sgRNA target site sequence
ggcactgcggctggaggtgg

SEQ ID NO: 25 - DNA, 20 bp, Artificial Sequence
  EMX1 sgRNA target site sequence
gagtccgagcagaagaagaa

SEQ ID NO: 26 - DNA, 20 bp, Artificial Sequence
  RNF2 sgRNA target site sequence
gtcatcttagtcattacctg

SEQ ID NO: 27 - DNA, 20 bp, Artificial Sequence
  RUNX1 sgRNA target site sequence
tcccctctgctggatacctc

SEQ ID NO: 28 - DNA, 137 bp, Artificial Sequence
  dIdC model sequence
  misc_feature (52)..(52): n is hypoxanthine deoxyribonucleotide
aactgattgcccgtctccgctcgctgggtgaacaactgaaccgtgatttcancatgacgaatgtggatgccgcagttggctccgtgtggcagagctgaaagaggagcttgatgacacgcaaccgggacatcacggat

SEQ ID NO: 29 - DNA, 139 bp, Artificial Sequence
  dIdT model sequence
  misc_feature (40)..(40): n is Hypoxanthine deoxyribonucleotide
aactgattgcccgtctccgctcgctgggtgaacaactganccgtgatgtcagcatgacgctacgcaaactggctgtcaagctccgtgtggcagagctgaaagaggagcttgatgacacgtcatggacgctacctcacag

SEQ ID NO: 30 - DNA, 139 bp, Artificial Sequence
  Nick model sequence
aactgattgcccgtctccgctcgctgggtgaacaactgaaccgtgatgtcagcatgacgaggccaacatacatgccttcgctccgtgtggcagagctgaaagaggagcttgatgacacggaatggcagagtcaaggagc

SEQ ID NO: 31 - DNA, 19 bp, Artificial Sequence
  qPCR Primer-1 for dIdC model sequence
aatgtggatgccgcagttg

SEQ ID NO: 32 - DNA, 19 bp, Artificial Sequence
  qPCR Primer-2 for dIdC model sequence
atccgtgatgtcccggttg

SEQ ID NO: 33 - DNA, 20 bp, Artificial Sequence
  qPCR Primer-1 for dIdT model sequence
ctacgcaaactggctgtcaa

SEQ ID NO: 34 - DNA, 20 bp, Artificial Sequence
  qPCR Primer-2 for dIdT model sequence
ctgtgaggtagcgtccatga

SEQ ID NO: 35 - DNA, 20 bp, Artificial Sequence
  qPCR Primer-1 for Nick model sequence
aggccaacatacatgccttc

SEQ ID NO: 36 - DNA, 20 bp, Artificial Sequence
  qPCR Primer-2 for Nick model sequence
gctccttgactctgccattc

SEQ ID NO: 37 - DNA, 23 bp, Artificial Sequence
  RUNX1 crRNA target site sequence
ttctcccctctgctggatacctc

SEQ ID NO: 38 - DNA, 23 bp, Artificial Sequence
  DYRK1A crRNA target site sequence
gaagcacatcaaggacattctaa